(19) United States
(12) Patent Application Publication
(10) Pub. No.: US 2020/0223925 A1
(43) Pub. Date: Jul. 16, 2020

(54) TUMOR-TARGETED SUPERAGONISTIC CD28 ANTIGEN BINDING MOLECULES

(71) Applicant: Hoffmann-La Roche Inc., Little Falls, NJ (US)

(72) Inventors: Stephan GASSER, Schlieren (CH); Guy GEORGES, Penzberg (DE); Thomas HOFER, Schlieren (CH); Christian KLEIN, Schlieren (CH); Jenny Tosca THOM, Schlieren (CH); Pablo UMAÑA, Schlieren (CH)

(73) Assignee: Hoffmann-La Roche Inc., Little Falls, NJ (US)

(21) Appl. No.: 16/721,217

(22) Filed: Dec. 19, 2019

(30) Foreign Application Priority Data
Dec. 21, 2018 (EP) 18215116.7

(51) Int. Cl. C07K 16/28; C07K 16/30; A61P 35/00

(52) U.S. Cl. CPC C07K 16/2818; C07K 16/3007; A61P 35/00; A61K 2039/505; C07K 2317/565; C07K 2317/55; C07K 2317/52; C07K 2317/31

(57) ABSTRACT

The present invention relates to tumor targeted superagonistic antigen binding molecules capable of multivalent binding to CD28, methods for their production, pharmaceutical compositions containing these antibodies, and methods of using the same.

### FIELD OF THE INVENTION

The present invention relates to tumor-targeted superagonistic CD28 antigen binding molecules, methods for their production, pharmaceutical compositions containing these molecules, and their use as immunomodulators in the treatment of cancer.

### BACKGROUND

Cancer immunotherapy is becoming an increasingly effective therapy option that can result in dramatic and durable responses in cancer types such as melanoma, non-small cell lung cancer and renal cell carcinoma. This is mostly driven by the success of several immune checkpoint blockades including anti-PD-1 (e.g. Keytruda, Merck; Opdivo, BMS), anti-CTLA-4 (e.g. Yervoy, BMS) and anti-PD-L1 (e.g. Tecentriq, Roche). These agents are likely to serve as standard of care for many cancer types, or as the backbone of combination therapies, however, only a fraction of patients (<25%) benefits from such therapies. Furthermore, various cancers (prostate cancer, colorectal cancer, pancreatic cancer, sarcomas, non-triple negative breast cancer etc.) present primary resistance to these immunomodulators. A number of reports indicate that the absence of pre-existing anti-tumor T cells contributes to the absence or poor response of some patients. In summary, despite impressive anti-cancer effects of existing immunotherapies, there is a clear medical need for addressing a large cancer patient population and for developing therapies that aim to induce and enhance novel tumor-specific T cell responses.

CD28 is the founding member of a subfamily of costimulatory molecules characterized by paired V-set immunoglobulin superfamily (IgSF) domains attached to single transmembrane domains and cytoplasmic domains that contain critical signaling motifs (Carreno and Collins, 2002). Other members of the subfamily include ICOS, CTLA-4, PD1, PD1H, TIGIT, and BTLA (Chen and Flies, 2013). CD28 expression is restricted to T cells and prevalent on all naïve and a majority of antigen-experienced subsets, including those that express PD-1 or CTLA-4. CD28 and CTLA-4 are highly homologous and compete for binding to the same B7 molecules CD80 and CD86, which are expressed on dendritic cells, B cells, macrophages, and tumor cells (Linsley et al., 1990). The higher affinity of CTLA-4 for the B7 family of ligands allows CTLA-4 to outcompete CD28 for ligand binding and suppress effector T cells responses (Engelhardt et al., 2006). In contrast, PD-1 was shown to inhibit CD28 signaling by in part dephosphorylating the cytoplasmic domain of CD28 (Hui et al., 2017). Ligation of CD28 by CD80 or CD86 on the surface of professional antigen-presenting cells is strictly required for functional de novo priming of naïve T cells, subsequent clonal expansion, cytokine production, target cell lysis, and formation of long-lived memory. Binding of CD28 ligands also promotes the expression of inducible co-stimulatory receptors such as OX-40, ICOS, and 4-1BB (reviewed in Acuto and Michel, 2003).

Upon ligation of CD28, a disulfide-linked homodimer, the membrane proximal YMNM motif and the distal PYAP motif have been shown to complex with several kinases and adaptor proteins (Boomer and Green, 2010). These motifs are important for the induction of IL2 transcription, which is mediated by the CD28-dependent activation of NFAT, AP-1, and NFκB family transcription factors (Fraser et al., 1991) (June et al., 1987) (Thompson et al., 1989). However, additional poorly characterized sites for phosphorylation and ubiquitination are found within the cytoplasmic domain of CD28.

As reviewed by (Esensten et al., 2016), CD28-initiated pathways have critical roles in promoting the proliferation and effector function of conventional T cells. CD28 ligation also promotes the anti-inflammatory function of regulatory T cells. CD28 co-stimulates T cells by in part augmenting signals from the T cell receptor, but was also shown to mediate unique signaling events (Acuto and Michel, 2003; Boomer and Green, 2010; June et al., 1987). Signals specifically triggered by CD28 control many important aspects of T cell function, including phosphorylation and other post-translational modifications of downstream proteins (e.g., PI3K mediated phosphorylation), transcriptional changes (eg. Bcl-xL expression), epigenetic changes (e.g. IL-2 promoter), cytoskeletal remodeling (e.g. orientation of the microtubule-organizing center) and changes in the glycolytic rate (e.g. glycolytic flux).

CD28-deficient mice have reduced responses to infectious pathogens, allograft antigens, graft-versus-host disease, contact hypersensitivity and asthma (Acuto and Michel, 2003). Lack of CD28-mediated co-stimulation results in reduced T cell proliferation in vitro and in vivo, in severe inhibition of germinal-centre formation and immunoglobulin isotype-class switching, reduced T helper (Th)-cell differentiation and the expression of Th2-type cytokines. CD4-dependent cytotoxic CD8+ T-cell responses are also affected. Importantly, CD28-deficient naïve T cells showed a reduced proliferative response particularly at lower antigen concentrations.

A growing body of literature supports the idea that engaging CD28 on T cells has anti-tumor potential. Recent evidence demonstrates that the anti-cancer effects of PD-L1/PD-1 and CTLA-4 checkpoint inhibitors depend on CD28 (Kamphorst et al., 2017; Tai et al., 2007). Clinical studies investigating the therapeutic effects of CTLA-4 and PD-1 blockade have shown exceptionally promising results in patients with advanced melanoma and other cancers. In addition, infusion of genetically engineered T cells expressing artificial chimeric T cell receptors comprising an extracellular antigen recognition domain fused to the intracellular TCR signaling domains (CD3z) and intracellular co-stimulatory domains (CD28 and/or 4-1BB domains) has shown high rates and durability of response in B cell cancers and other cancers.

CD28 agonistic antibodies can be divided into two categories: (i) CD28 superagonistic antibodies and (ii) CD28 conventional agonistic antibodies. Normally, for the activation of naïve T cells both engagement of the T cell antigen receptor (TCR, signal 1) and costimulatory signaling by CD28 (signal 2) is required. CD28 Superagonists (CD28SA) are CD28-specific monoclonal antibodies, which are able to autonomously activate T cells without overt T cell receptor engagement (Hunig, 2012). In rodents, CD28SA activates conventional and regulatory T cells. CD28SA antibodies are therapeutically effective in multiple models of autoimmunity, inflammation and transplantation. However, a phase I study of the human CD28SA antibody TGN1412 resulted in a life-threatening cytokine storm in 2006. Follow-up studies have suggested that the toxicity was caused by dosing errors due to differences in the CD28 responsiveness of human T cells and T cells of preclinical animal models. TGN1412 is currently being re-evaluated in an open-label, multi-center dose escalation study in RA patients and patients with metastatic or unresectable advanced solid malignancies. CD28 conventional agonistic antibodies, such as clone 9.3, mimic CD28 natural ligands and are only able to enhance T cell activation in presence of a T cell receptor signal. Published insights indicate that the binding epitope of the antibody has a major impact on whether the agonistic antibody is a superagonist or a conventional agonist (Beyersdorf et al., 2005). The superagonistic TGN1412 binds to a lateral motif of CD28, while the conventional agonistic molecule 9.3 binds close to the ligand binding epitope. As a consequence of the different binding epitopes, superagonistic and conventional agonistic antibodies differ in their ability to form linear complexes of CD28 molecules on the surface of T cells. Precisely, TGN1412 is able to efficiently form linear arrays of CD28, which presumably leads to aggregated signaling components which are sufficient to surpass the threshold for T cell activation. The conventional agonist 9.3, on the other hand, leads to complexes which are not linear in structure. An attempt to convert conventional agonistic binders based on the 9.3 clone has been previously published (Otz et al., 2009), using a recombinant bi-specific single-chain antibody directed to a melanoma-associated proteoglycan and CD28. The reported bispecific single chain antibody exerted “supra-agonistic” activity despite the use of a conventional CD28 agonistic binder 9.3, based in the intrinsic tendency of bispecific single chain antibodies to form multimeric constructs. These constructs, however, rely on stable and consistent multimerization.

### SUMMARY OF THE INVENTION

The present invention describes tumor-targeted superagonistic CD28 antigen binding molecules which achieve a tumor-dependent autonomous T cell activation and tumor cell killing without the necessity to form multimers. These CD28 antigen binding molecules are characterized in that they are capable of multivalent binding to CD28 and in that they comprise at least one antigen binding domain capable of specific binding to a tumor-associated antigen such as Fibroblast Activation Protein (FAP) or Carcinoembryonic Antigen (CEA). Furthermore, they possess an Fc domain composed of a first and a second subunit capable of stable association comprising one or more amino acid substitution that reduces the binding affinity of the antigen binding molecule to an Fc receptor and/or effector function. Fc receptor-mediated cross-linking is thereby abrogated and tumor-specific activation is achieved by cross-linking through binding of the at least one antigen binding domain capable of specific binding to a tumor-associated antigen to its antigen.

Thus, the invention provides a superagonistic CD28 antigen binding molecule, which is capable of multivalent binding to CD28 and comprises

(a) two or more antigen binding domains capable of specific binding to CD28,
(b) at least one antigen binding domain capable of specific binding to a tumor-associated antigen, and
(c) an Fc domain composed of a first and a second subunit capable of stable association comprising one or more amino acid substitution that reduces the binding affinity of the antigen binding molecule to an Fc receptor and/or effector function.

The invention thus relates to a CD28 antigen binding molecule that is capable to induce T cell proliferation and cytokine secretion without prior T cell activation. It will however only induce T cell proliferation and cytokine secretion without prior T cell activation when it binds to a tumor-associated antigen as cross-linking through binding of the at least one antigen binding domain capable of specific binding to a tumor-associated antigen to its antigen is required because the CD28 antigen binding molecule is lacking Fc receptor and/or effector function.

In one aspect, a superagonistic CD28 antigen binding molecule as defined below is provided, wherein the Fc domain is an IgG, particularly an IgG1 Fc domain or an IgG4 Fc domain. In one particular aspect, the Fc domain composed of a first and a second subunit capable of stable association is an IgG1 Fc domain. In one aspect, the Fc domain comprises the amino acid substitutions L234A and L235A (numbering according to Kabat EU index). In one aspect, the Fc domain is of human IgG1 subclass and comprises the amino acid mutations L234A, L235A and P329G (numbering according to Kabat EU index).

In one aspect, provided is a superagonistic CD28 antigen binding molecule as defined herein before, wherein each of the antigen binding domains capable of specific binding to CD28 comprises

(i) a heavy chain variable region (VHCD28) comprising a heavy chain complementary determining region CDR-H1 of SEQ ID NO: 20, a CDR-H2 of SEQ ID NO: 21, and a CDR-H3 of SEQ ID NO: 22, and a light chain variable region (V_{L}CD28) comprising a light chain complementary determining region CDR-L1 of SEQ ID NO: 23, a CDR-L2 of SEQ ID NO: 24 and a CDR-L3 of SEQ ID NO: 25; or
(ii) a heavy chain variable region (VHCD28) comprising a CDR-H1 of SEQ ID NO: 36, a CDR-H2 of SEQ ID NO: 37, and a CDR-H3 of SEQ ID NO: 38, and a light chain variable region (V_{L}CD28) comprising a CDR-L1 of SEQ ID NO: 39, a CDR-L2 of SEQ ID NO: 40 and a CDR-L3 of SEQ ID NO: 41.

In one aspect, each of the antigen binding domains capable of specific binding to CD28 of the superagonistic CD28 antigen binding molecule comprises a heavy chain variable region (VHCD28) comprising a CDR-H1 of SEQ ID NO: 36, a CDR-H2 of SEQ ID NO: 37, and a CDR-H3 of SEQ ID NO: 38, and a light chain variable region (V_{L}CD28) comprising a CDR-L1 of SEQ ID NO: 39, a CDR-L2 of SEQ ID NO: 40 and a CDR-L3 of SEQ ID NO: 41.

In another aspect, each of the antigen binding domains capable of specific binding to CD28 of the superagonistic CD28 antigen binding molecule comprises a heavy chain variable region (VHCD28) comprising a CDR-H1 of SEQ ID NO: 20, a CDR-H2 of SEQ ID NO: 21, and a CDR-H3 of SEQ ID NO: 22, and a light chain variable region (V_{L}CD28) comprising a CDR-L1 of SEQ ID NO: 23, a CDR-L2 of SEQ ID NO: 24 and a CDR-L3 of SEQ ID NO: 25.

Furthermore, provided is a superagonistic CD28 antigen binding molecule as defined herein before, wherein each of the antigen binding domains capable of specific binding to CD28 comprises a heavy chain variable region (VHCD28) comprising an amino acid sequence that is at least about 95%, 96%, 97%, 98%, 99% or 100% identical to the amino acid sequence of SEQ ID NO:26, and a light chain variable region (V_{L}CD28) comprising an amino acid sequence that is at least about 95%, 96%, 97%, 98%, 99% or 100% identical to the amino acid sequence of SEQ ID NO:27.

In a further aspect, a superagonistic CD28 antigen binding molecule is provided, wherein each of the antigen binding domains capable of specific binding to CD28 comprises a heavy chain variable region (VHCD28) comprising an amino acid sequence selected from the group consisting of SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50 and SEQ ID NO:51, and a light chain variable region (V_{L}CD28) comprising an amino acid sequence selected from the group consisting of SEQ ID NO:27, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60 and SEQ ID NO:61.

In another aspect, provided is a superagonistic CD28 antigen binding molecule, wherein each of the antigen binding domains capable of specific binding to CD28 comprises

(a) a heavy chain variable region (V_{H}CD28) comprising the amino acid sequence of SEQ ID NO:47 and a light chain variable region (V_{L}CD28) comprising the amino acid sequence of SEQ ID NO:54, or

(b) a heavy chain variable region (V_{H}CD28) comprising the amino acid sequence of SEQ ID NO:47 and a light chain variable region (V_{L}CD28) comprising the amino acid sequence of SEQ ID NO:27, or

(c) a heavy chain variable region (V_{H}CD28) comprising the amino acid sequence of SEQ ID NO:51 and a light chain variable region (V_{L}CD28) comprising the amino acid sequence of SEQ ID NO:61, or

(d) a heavy chain variable region (V_{H}CD28) comprising the amino acid sequence of SEQ ID NO:46 and a light chain variable region (V_{L}CD28) comprising the amino acid sequence of SEQ ID NO:53, or

(e) a heavy chain variable region (V_{H}CD28) comprising the amino acid sequence of SEQ ID NO:46 and a light chain variable region (V_{L}CD28) comprising the amino acid sequence of SEQ ID NO:54, or

(f) a heavy chain variable region (V_{H}CD28) comprising the amino acid sequence of SEQ ID NO:46 and a light chain variable region (V_{L}CD28) comprising the amino acid sequence of SEQ ID NO:59, or

(g) a heavy chain variable region (V_{H}CD28) comprising the amino acid sequence of SEQ ID NO:46 and a light chain variable region (V_{L}CD28) comprising the amino acid sequence of SEQ ID NO:27, or

(h) a heavy chain variable region (V_{H}CD28) comprising the amino acid sequence of SEQ ID NO:43 and a light chain variable region (V_{L}CD28) comprising the amino acid sequence of SEQ ID NO:27, or

(i) a heavy chain variable region (V_{H}CD28) comprising the amino acid sequence of SEQ ID NO:42 and a light chain variable region (V_{L}CD28) comprising the amino acid sequence of SEQ ID NO:53, or

(j) a heavy chain variable region (V_{H}CD28) comprising the amino acid sequence of SEQ ID NO:42 and a light chain variable region (V_{L}CD28) comprising the amino acid sequence of SEQ ID NO:59, or

(k) a heavy chain variable region (V_{H}CD28) comprising the amino acid sequence of SEQ ID NO:42 and a light chain variable region (V_{L}CD28) comprising the amino acid sequence of SEQ ID NO:27.

In one particular aspect, a superagonistic CD28 antigen binding molecule is provided, wherein each of the antigen binding domains capable of specific binding to CD28 comprises a heavy chain variable region (V_{H}CD28) comprising the amino acid sequence of SEQ ID NO:47 and a light chain variable region (V_{L}CD28) comprising the amino acid sequence of SEQ ID NO:54.

In another particular aspect, a superagonistic CD28 antigen binding molecule is provided, wherein each of the antigen binding domains capable of specific binding to CD28 comprises a heavy chain variable region (V_{H}CD28) comprising the amino acid sequence of SEQ ID NO:46 and a light chain variable region (V_{L}CD28) comprising the amino acid sequence of SEQ ID NO:53.

In a further particular aspect, a superagonistic CD28 antigen binding molecule is provided, wherein each of the antigen binding domains capable of specific binding to CD28 comprises a heavy chain variable region (V_{H}CD28) comprising the amino acid sequence of SEQ ID NO:42 and a light chain variable region (V_{L}CD28) comprising the amino acid sequence of SEQ ID NO:27.

In a further aspect, provided is a superagonistic CD28 antigen binding molecule as defined herein before, wherein each of the antigen binding domains capable of specific binding to CD28 is a Fab fragment.

In one aspect, a superagonistic CD28 antigen binding molecule is provided, wherein the antigen binding domain capable of specific binding to a tumor-associated antigen is an antigen binding domain capable of specific binding to Carcinoembryonic Antigen (CEA).

In one aspect, provided is a superagonistic CD28 antigen binding molecule as described herein, wherein the antigen binding domain capable of specific binding to CEA comprises a heavy chain variable region (V_{H}CEA) comprising (i) CDR-H1 comprising the amino acid sequence of SEQ ID NO:127, (ii) CDR-H2 comprising the amino acid sequence of SEQ ID NO:128, and (iii) CDR-H3 comprising the amino acid sequence of SEQ ID NO:129, and a light chain variable region (V_{L}CEA) comprising (iv) CDR-L1 comprising the amino acid sequence of SEQ ID NO:130, (v) CDR-L2 comprising the amino acid sequence of SEQ ID NO:131, and (vi) CDR-L3 comprising the amino acid sequence of SEQ ID NO:132. Particularly, the antigen binding domain capable of specific binding to CEA comprises a heavy chain variable region (V_{H}CEA) comprising an amino acid sequence that is at least about 95%, 96%, 97%, 98%, 99% or 100% identical to the amino acid sequence of SEQ ID NO:133, and a light chain variable region (V_{L}CEA) comprising an amino acid sequence that is at least about 95%, 96%, 97%, 98%, 99% or 100% identical to the amino acid sequence of SEQ ID NO:134.

In another aspect, a superagonistic CD28 antigen binding molecule is provided, wherein the antigen binding domain capable of specific binding to a tumor-associated antigen is an antigen binding domain capable of specific binding to Fibroblast Activation Protein (FAP).

In one aspect, provided is a superagonistic CD28 antigen binding molecule as described herein, wherein the antigen binding domain capable of specific binding to FAP comprises

(a) a heavy chain variable region (V_{H}FAP) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}FAP) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17, or

(b) a heavy chain variable region (V_{H}FAP) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}FAP) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9. In particular, the antigen binding domain capable of specific binding to FAP comprises a heavy chain variable region (V_{H}FAP) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}FAP) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17.

In one aspect, a superagonistic CD28 antigen binding molecule is provided, wherein the antigen binding domain capable of specific binding to FAP comprises (a) a heavy chain variable region (V_{H}FAP) comprising an amino acid sequence that is at least about 95%, 96%, 97%, 98%, 99% or 100% identical to the amino acid sequence of SEQ ID NO:18, and a light chain variable region (V_{L}FAP) comprising an amino acid sequence that is at least about 95%, 96%, 97%, 98%, 99% or 100% identical to the amino acid sequence of SEQ ID NO:19, or (b) a heavy chain variable region (V_{H}FAP) comprising an amino acid sequence that is at least about 95%, 96%, 97%, 98%, 99% or 100% identical to the amino acid sequence of SEQ ID NO:10, and a light chain variable region (V_{L}FAP) comprising an amino acid sequence that is at least about 95%, 96%, 97%, 98%, 99% or 100% identical to the amino acid sequence of SEQ ID NO:11. Particularly, the antigen binding domain capable of specific binding to FAP comprises a heavy chain variable region (V_{L}FAP) comprising the amino acid sequence of SEQ ID NO:18 and a light chain variable region (V_{L}FAP) comprising the amino acid sequence of SEQ ID NO:19.

In another aspect, provided is a superagonistic CD28 antigen binding molecule as described herein, comprising

(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and

(b) a VH and VL domain capable of specific binding to a tumor-associated antigen, wherein the VH domain is connected via a peptide linker to the C-terminus of one of the two heavy chains and wherein the VL domain is connected via a peptide linker to the C-terminus of the second heavy chain.

In a further aspect, provided is a superagonistic CD28 antigen binding molecule as described herein, comprising

(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and

(b) a crossFab fragment capable of specific binding to a tumor-associated antigen which is connected via a peptide linker to the C-terminus of one of the two heavy chains.

In another aspect, a superagonistic CD28 antigen binding molecule as disclosed herein is provided, comprising

(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and

(b) two crossFab fragments capable of specific binding to a tumor-associated antigen, wherein one crossFab fragment is connected via a peptide linker to the C-terminus of one of the two heavy chains and wherein the other crossFab fragment is connected via a peptide linker to the C-terminus of the second heavy chain.

According to another aspect of the invention there is provided one or more isolated polynucleotide encoding an antibody or bispecific antigen binding molecule of the invention. The invention further provides one or more expression vector(s) comprising the isolated polynucleotide(s) of the invention, and a host cell comprising the isolated polynucleotide(s) or the expression vector(s) of the invention. In some aspects, the host cell is a eukaryotic cell, particularly a mammalian cell. In another aspect, provided is a method of producing a superagonistic CD28 antigen binding molecule as described herein comprising culturing the host cell of the invention under conditions suitable for the expression of the superagonistic CD28 antigen binding molecule. Optionally, the method also comprises recovering the superagonistic CD28 antigen binding molecule. The invention also encompasses a superagonistic CD28 antigen binding molecule produced by the method of the invention.

The invention further provides a pharmaceutical composition comprising a superagonistic CD28 antigen binding molecule of the invention and at least one pharmaceutically acceptable excipient. In one aspect, the pharmaceutical composition is for use in the treatment of cancer.

Also encompassed by the invention are methods of using the superagonistic CD28 antigen binding molecule and the pharmaceutical composition of the invention. In one aspect the invention provides a superagonistic CD28 antigen binding molecule or a pharmaceutical composition according to the invention for use as a medicament. In one aspect is provided a superagonistic CD28 antigen binding molecule or pharmaceutical composition according to the invention for use in the treatment of a disease. In a specific aspect, the disease is cancer. In another aspect is provided a superagonistic CD28 antigen binding molecule or pharmaceutical composition according to the invention for use in the treatment of cancer, wherein the superagonistic CD28 antigen binding molecule is administered in combination with a chemotherapeutic agent, radiation therapy and/or other agents for use in cancer immunotherapy.

Also provided is the use of a superagonistic CD28 antigen binding molecule according to the invention in the manufacture of a medicament for the treatment of a disease; as well as a method of treating a disease in an individual, comprising administering to said individual a therapeutically effective amount of a superagonistic CD28 antigen binding molecule according to the invention or a composition comprising the superagonistic CD28 antigen binding molecule according to the invention in a pharmaceutically acceptable form. In a specific aspect, the disease is cancer. In another aspect, provided is the use of a superagonistic CD28 antigen binding molecule according to the invention in the manufacture of a medicament for the treatment of a disease, wherein the treatment comprises co-administration with a chemotherapeutic agent, radiation therapy and/or other agents for use in cancer immunotherapy. In a further aspect, provided is a method of treating a disease in an individual, comprising administering to said individual a therapeutically effective amount of a superagonistic CD28 antigen binding molecule according to the invention or a composition comprising the superagonistic CD28 antigen binding molecule according to the invention in a pharmaceutically acceptable form, wherein the method comprises co-administration with a chemotherapeutic agent, radiation therapy and/or other agents for use in cancer immunotherapy. Also provided is a method of inhibiting the growth of tumor cells in an individual comprising administering to the individual an effective amount of the superagonistic CD28 antigen binding molecule according to the invention, or a composition comprising the superagonistic CD28 antigen binding molecule according to the invention in a pharmaceutically acceptable form, to inhibit the growth of the tumor cells. In any of the above aspects, the individual preferably is a mammal, particularly a human.

### BRIEF DESCRIPTION OF THE DRAWINGS

In FIGS. 1A to 1L schematic illustrations of the molecules as described are shown. FIG. 1A shows the CD28 agonistic antibody CD28(SA) in its huIgG4 isoform (TGN1412). FIG. 1B illustrates the CD28(SA) agonistic antibody as hu IgG1 PGLALA isotype (“Fc silent”).

Bispecific FAP-CD28 antigen binding molecules in 1+1 format, 1+2 format, 2+2 format and 1+4 format are shown in FIGS. 1C, 1D, 1E and IF, respectively.

Bispecific CEA-CD28 antigen binding molecules in 1+2 format, 2+2 format and 1+1 format are shown in FIGS. 1G, 1H and 1J, respectively.

FIG. 1I shows a schematic illustration of the CD28 agonistic antibody variants as monovalent hu IgG1 PGLALA isotype (“Fc silent”).

Trispecific CEA-FAP-CD28 antigen binding molecules in 1+1+2 format are shown in two alternative formats in FIGS. 1K and 1L, respectively.

FIGS. 2A, 2B, 2C, 2D and 2E relate to the binding of CD28 agonistic antibodies and FAP-CD28 antigen binding molecules to human CD28 or human FAP on cells. Shown is the binding of CD28(SA) in it IgG4 isoform vs. hu IgG1 PGLALA isotype ti human CD28 in FIG. 2A and the binding of different FAP-CD28 molecules to human CD28 (FIG. 2B) and human FAP (FIG. 2C) on cells. Median fluorescence intensities of binding of different CD28 agonistic antibodies or anti-DP47 targeted molecules to CHO cells expressing human CD28 (parental cell line CHO-k1 ATCC # CCL-61, modified to stably overexpress human CD28) or 3T3 cells expressing human FAP (NIH/3T3 cell line (ATCC CRL-1658)) was assessed by flow cytometry. Depicted are technical triplicates with SEM. A comparison of FAP(4B9)-CD28(SA) antigen binding molecules (Molecules D, E and F as described in Example 1) is shown in FIG. 2D (binding to human CD28) and FIG. 2E (binding to human FAP).

The alignment of the variable domains of CD28(SA) and variants thereof is shown in FIGS. 3A to 3D. Alignment of the CD28(SA) VH domain and variants thereof in order to remove cysteine 50 and to reduce the affinity of the resulting anti-CD28 binders to different degrees is shown in FIGS. 3A and 3B. Of note, in VH variants i and j, the CDRs of CD28(SA) were grafted from an IGHV1-2 framework into an IGHV3-23 framework (FIG. 3B). In FIGS. 3C and 3D, alignment of the CD28(SA) VL domain and variants thereof in order to reduce the affinity of the resulting anti-CD28 binders to different degrees is shown. In variant t, the CDRs were grafted into the framework sequence of the trastuzumab (Herceptin) VL sequence.

In FIGS. 4A to 4C the binding of affinity-reduced CD28 agonistic antibody variants in monovalent IgG formats from supernatants to human CD28 on cells is shown. Median fluorescence intensities of binding to CHO cells expressing human CD28 (parental cell line CHO-k1 ATCC # CCL-61, modified to stably overexpress human CD28) compared to the negative control (anti-DP47) and the original TGN1412, were assessed by flow cytometry. The binding curves of variants 1-10 are shown in FIG. 4A, those of variants 11 to 22 in FIG. 4B and those of variants 23 to 31 in FIG. 4C. Depicted are technical duplicates with SD.

In FIGS. 4D and 4E, the binding of FAP-targeted bispecific CD28 agonistic antibody variants in huIgG1 PG-LALA 1+1 format with selected affinity-reduced CD28 agonistic antibody variants to human CD28 on cells is shown. The binding curves of bispecific 1+1 constructs with variants 8, 11, 12, 15, 16 and 17 are shown in FIG. 4D, whereas the binding curves of bispecific 1+1 constructs with variants 19, 23, 25, 27 and 29 are shown in FIG. 4E. Selected binders were chosen based on affinities for production in a 1+1, bispecific FAP-targeted format. Shown are median fluorescence intensities of binding to CHO cells expressing human CD28 (parental cell line CHO-k1 ATCC # CCL-61, modified to stably overexpress human CD28) compared to the negative control (anti-DP47) and the original TGN1412 (Molecule A), assessed by flow cytometry.

The in vitro potency of selected FAP-targeted bispecific CD28 agonistic antibody variants in huIgG1 PG-LALA 1+1 format is illustrated in FIGS. 4F and 4G. T cells were incubated with MCSP- and FAP-expressing MV3 melanoma cells for 5 days in the presence of limiting concentration of MCSP-TCB (5 pM, P1AD2189) and increasing concentration of FAP-CD28 constructs. In FIG. 4F is shown the CFSE-dilution as measure for T cell proliferation of CD8 T cells, assessed by flow cytometry. Error bars show SEM, graphs depict technical triplicates of representative results from 2 donors. In FIG. 4G is shown the correlation of K_{D} (nM) of the CD28 binder variant in relation to potency by area under the curve of (a) as % of the parental TGN1412 clone (CD28(SA)).

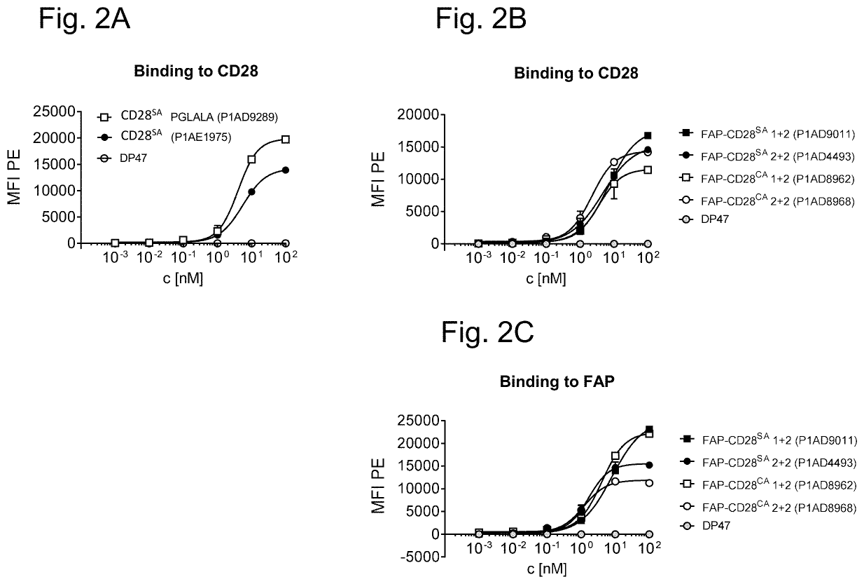

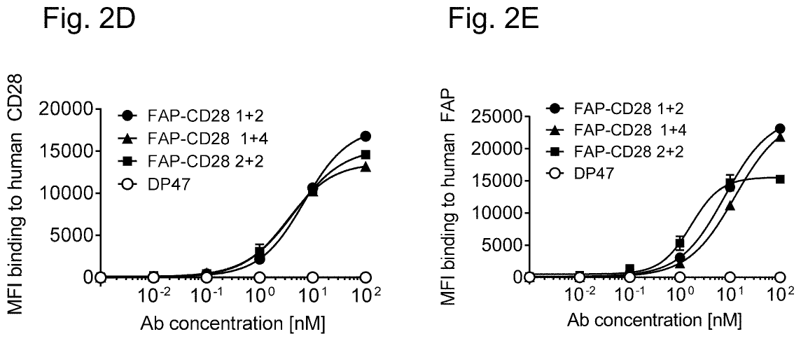

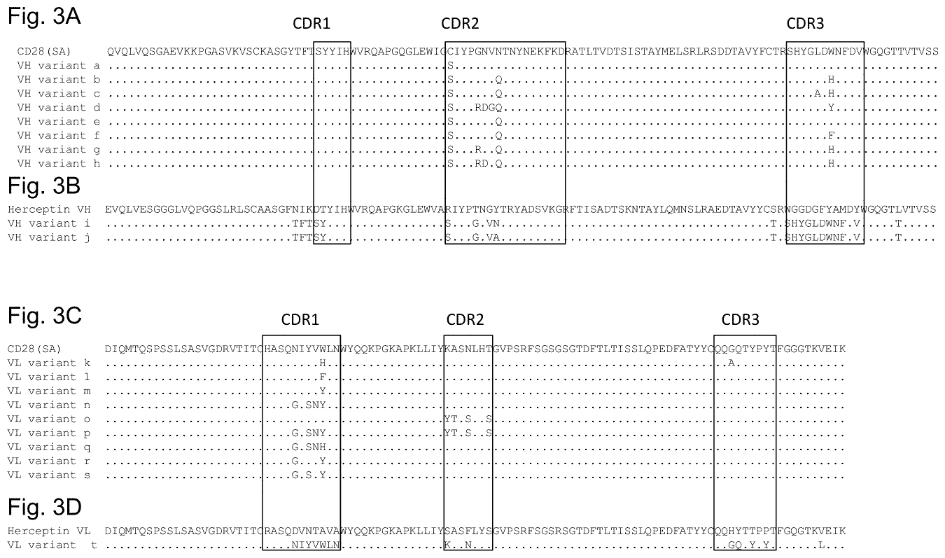

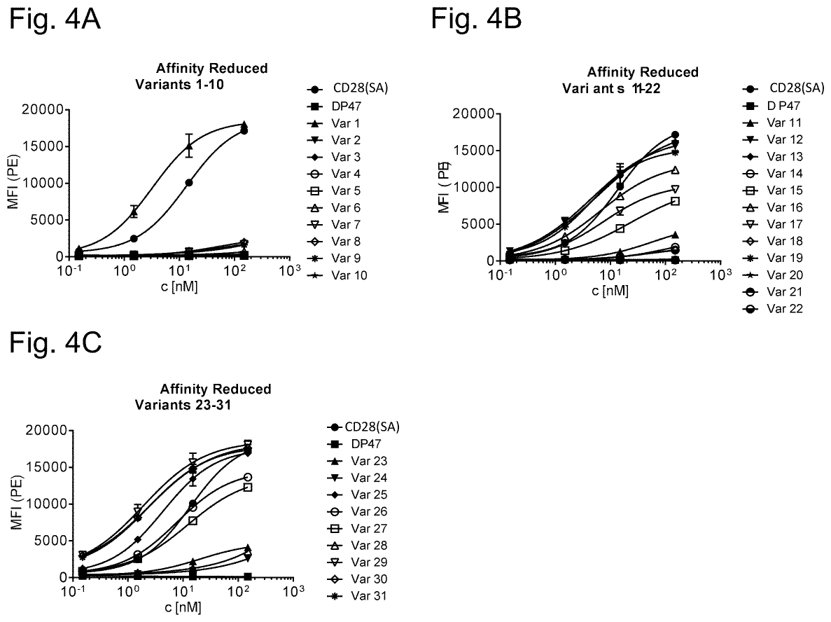

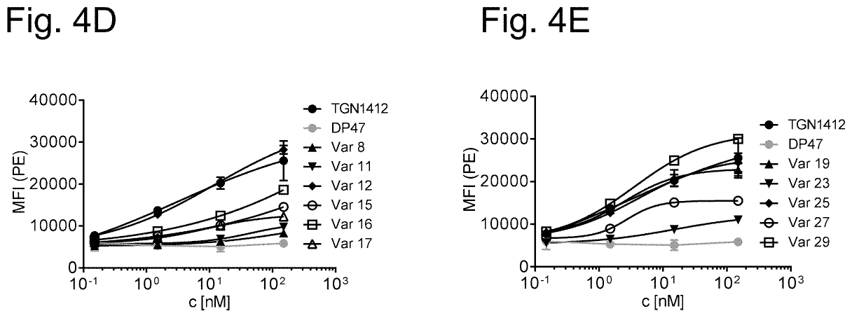

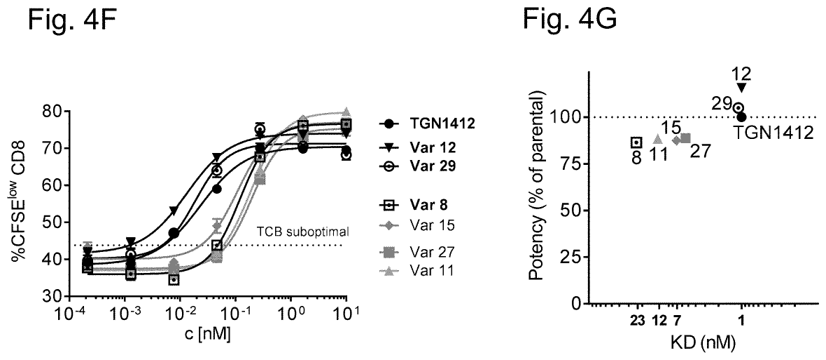

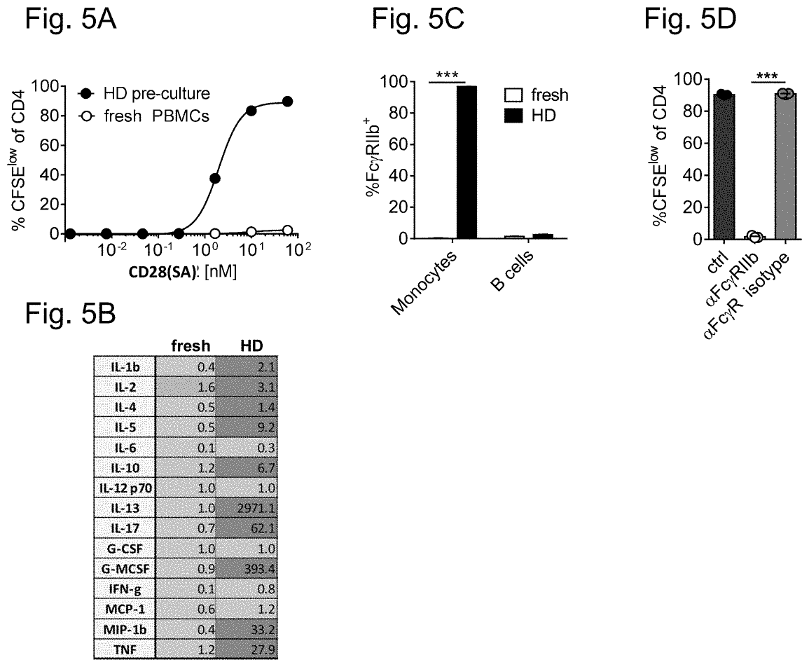

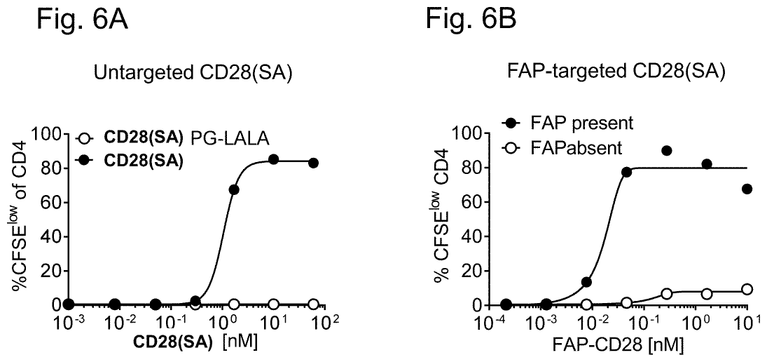

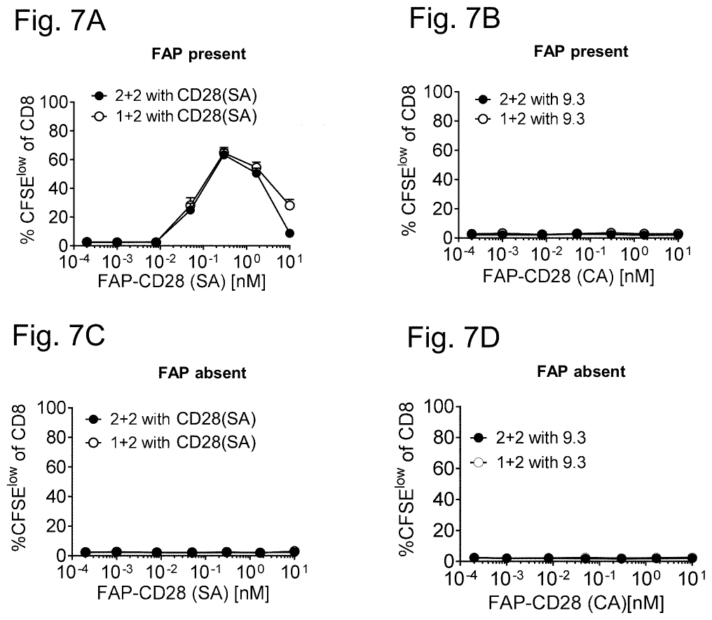

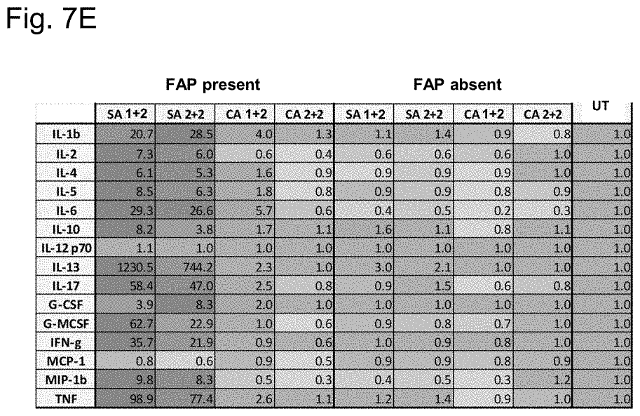

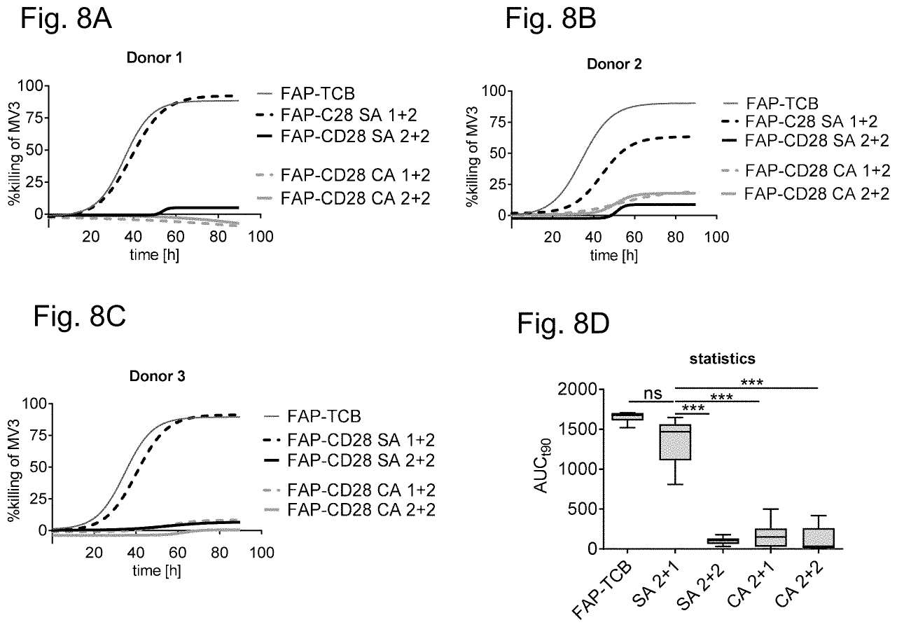

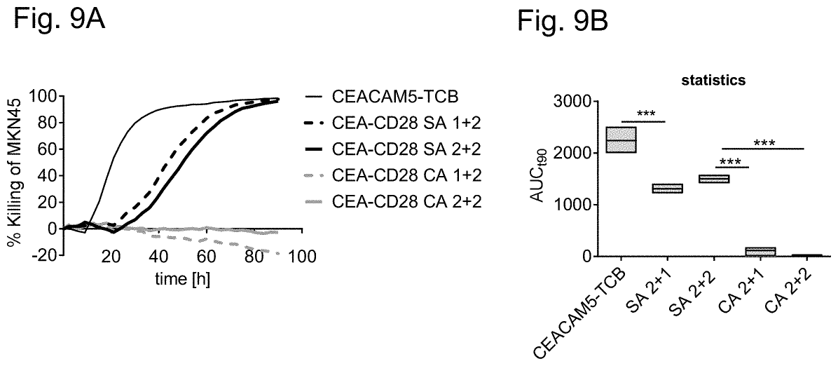

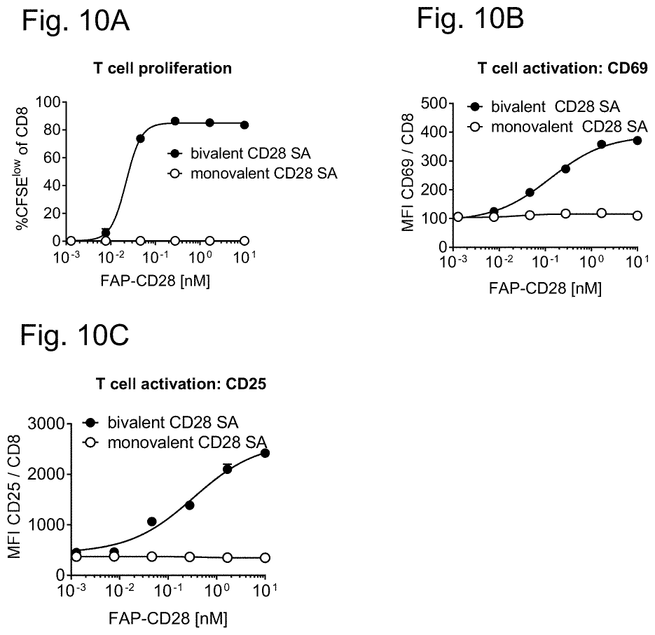

FIGS. 5A to 5D refer to the establishment of high-density (HD) pre-culture and mode of action of CD28(SA). PBMC T cells were either pre-cultured at high density (HD) for 2 days or used fresh from PBMC isolation and stimulated with increasing concentrations of CD28(SA). Depicted is CFSE-dilution as proxy for T cell proliferation after 5 days of stimulation with CD28(SA) (Molecule A, P1AE1975) (FIG. 5A) and cytokine secretion after 2 days (FIG. 5B) of stimulation. FIG. 5C shows the percentage of FcγRIIb expression in PBMC monocytes and B cells before and after 2 days HD PBMC pre-culture, assessed by flow cytometry. FIG. 5D: HD pre-cultured PBMCs were co-cultured with CD28(SA) for 5 days in presence or absence of an FcγRIIb blocking antibody or isotype control and percentage of CFSE-dilution of CD4 T cells was assessed by flow cytometry. Graphs are representative of at least 6 donors (FIGS. 5A, 5B) and 2 donors (FIGS. 5C, 5D), each assessed in independent experiments. The graphs show technical triplicates. Error bars indicate SEM. Statistical analysis was performed by student's t-test. ***: p<0.001. Superagonism of CD28(SA) IgG4 depends on cross-linking to FcγRIIb.

In FIGS. 6A and 6B the T cell proliferation, i.e. CFSE-dilution of CD4 T cells after 5 days of stimulation with either original Fc wild-type IgG4 CD28(SA) (P1AE1975) or CD28(SA) bearing the P329G-LALA mutation (P1AD9289) is shown. T cells were pre-cultured at high density for 2 days. Graphs are representative of at least 3 independent experiments. Technical triplicates are shown. Fc-silencing abolishes superagonism in TGN1412. Adding a tumor-targeting moiety to Fc-silenced TGN1412 restores superagonism, which is then dependent on the presence of the tumor-target.

In FIGS. 7A, 7B, 7C and 7D a comparison of FAP-targeted CD28 agonists in different formats (2+2 and 1+2) and with superagonistic (CD28(SA)) binders and conventional agonistic binders (9.3, CD28(CA)) is shown. FAP-targeted CD28 agonists with conventional CD28 agonistic binders do not function as superagonists. PBMC T cells were co-cultured with 3T3-huFAP cells (FAP present) in the presence of increasing concentrations of the FAP-CD28 formats with superagonistic binders (SA, FIG. 7A) or conventional agonistic binders (9.3, FIG. 7B) for 5 days. T cell proliferation is shown. PBMC T cells were then also co-cultured with 3T3 WT cells (FAP absent), in the presence of increasing concentrations of the FAP-CD28 formats with superagonistic binders (SA, FIG. 7C) or conventional agonistic binders (9.3, FIG. 7D) for 5 days. Depicted is CFSE-dilution as measure for T cell proliferation of CD8 T cells, assessed by flow cytometry on day 5 post stimulation. Graphs show cumulative data from 3 donors in 3 independent experiments. Error bars show SEM. In the same experimental setup also cytokines were measured from supernatants after 2 days of co-culture. The values are provided in FIG. 7E.

The ability of FAP-CD28 in various formats with either superagonistic CD28(SA) binders or conventional agonistic binders (CD28(CA)) to induce killing of FAP-expressing RFP-MV3 melanoma cells was assessed over the course of 90 h by live cell imaging using the IncuCyte technology. All molecules including the FAP-TCB (P1AD4645) were used at 10 nM. FIGS. 8A, 8B and 8C show representative results from three donors with technical triplicates, respectively. FIG. 8D shows the cumulative results expressed as area under the curve (AUC) at t=90 h of 3 donors from 3 independent experiments. Boxes display 25th-75th percentiles, whiskers display min to max. Statistical analysis was performed by paired 1-way ANOVA. ***: p<0.001, ns: not significant.

A comparison of CEA-targeted CD28 agonistis in different formats with superagonistic and conventional agonistic binders is shown in FIGS. 9A and 9B. The ability of CEA-CD28 in various formats with either superagonistic CD28(SA) binders or conventional agonistic binders (CD28(CA)) to induce killing of CEA-expressing RFP⁺ MKN45 gastric cancer cells was assessed over the course of 90 h by live cell imaging using the IncuCyte technology. All molecules including the CEACAM5-TCB (P1AD5299) were used at 10 nM. FIG. 9A shows representative results from one donor with technical triplicates. FIG. 9B shows the statistical analysis of technical triplicates expressed as area under the curve (AUC) at t=90 h of 1 donor in 1 experiment. Boxes display 25th-75th percentiles, whiskers display min to max. Statistical analysis was performed by paired 1-way ANOVA. ***: p<0.001. It is shown that CEA-targeted CD28 agonists with conventional CD28 agonistic binders do not behave superagonistically.

In FIGS. 10A, 10B and 10C it is shown that targeted CD28 agonists with monovalent superagonistic binders are not functionally superagonistic. PBMC T cells were co-cultured for 5 days with 3T3-huFAP cells in presence of increasing concentrations of FAP-CD28 with bivalent CD28 binders (P1AD9011, closed circles) or FAP-CD28 with monovalency for CD28 binding (P1AD4492, open circles). In FIG. 10A CFSE-dilution of CD8 T cells is shown. Furthermore, activation of T cells was assessed by detection of activation markers CD69 (FIG. 10B) and CD25 (FIG. 10C) by flow cytometry. Mean fluorescent intensity (MFI) of CD69 and CD25 stainings are shown at 5 days post stimulation. Technical triplicates from 1 donor are shown, error bars indicate SEM. It is shown that TGN1412-like superagonism requires multivalent CD28 binding.

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, multispecific antibodies (e.g., bispecific antibodies), antibody fragments and scaffold antigen binding proteins.

As used herein, the term “antigen binding domain that binds to a tumor-associated antigen” or “moiety capable of specific binding to a tumor-associated antigen” refers to a polypeptide molecule that specifically binds to an antigenic determinant. In one aspect, the antigen binding domain is able to activate signaling through its target cell antigen. In a particular aspect, the antigen binding domain is able to direct the entity to which it is attached (e.g. the CD28 superagonist) to a target site, for example to a specific type of tumor cell or tumor stroma bearing the antigenic determinant. Antigen binding domains capable of specific binding to a target cell antigen include antibodies and fragments thereof as further defined herein. In addition, antigen binding domains capable of specific binding to a target cell antigen include scaffold antigen binding proteins as further defined herein, e.g. binding domains which are based on designed repeat proteins or designed repeat domains (see e.g. WO 2002/020565).

In relation to an antigen binding molecule, i.e. an antibody or fragment thereof, the term “antigen binding domain that binds to a target cell antigen” refers to the part of the molecule that comprises the area which specifically binds to and is complementary to part or all of an antigen. An antigen binding domain capable of specific antigen binding may be provided, for example, by one or more antibody variable domains (also called antibody variable regions). Particularly, an antigen binding domain capable of specific antigen binding comprises an antibody light chain variable region (VL) and an antibody heavy chain variable region (VH). In another aspect, the “antigen binding domain capable of specific binding to a target cell antigen” can also be a Fab fragment or a crossFab fragment.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells or on the same cell.

The term “valent” as used within the current application denotes the presence of a specified number of binding sites specific for one distinct antigenic determinant in an antigen binding molecule that are specific for one distinct antigenic determinant. As such, the terms “bivalent”, “tetravalent”, and “hexavalent” denote the presence of two binding sites, four binding sites, and six binding sites specific for a certain antigenic determinant, respectively, in an antigen binding molecule. In particular aspects of the invention, the bispecific antigen binding molecules according to the invention can be monovalent for a certain antigenic determinant, meaning that they have only one binding site for said antigenic determinant or they can be bivalent or tetravalent for a certain antigenic determinant, meaning that they have two binding sites or four binding sites, respectively, for said antigenic determinant.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CHL CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, crossFab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a variable light chain (VL) domain and a constant domain of a light chain (CL), and a variable heavy chain (VH) domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments in which the cysteine residue(s) of the constant domains bear a free thiol group. Pepsin treatment yields an F(ab′)₂ fragment that has two antigen-combining sites (two Fab fragments) and a part of the Fc region.

The term “crossFab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable (VL) domain and the heavy chain constant domain (CH1), and a peptide chain composed of the heavy chain variable domain (VH) and the light chain constant domain (CL). This crossover Fab molecule is also referred to as CrossFab_{(VLVH)}. On the other hand, when the constant regions of the Fab heavy and light chain are exchanged, the crossover Fab molecule comprises a peptide chain composed of the heavy chain variable domain (VH) and the light chain constant domain (CL), and a peptide chain composed of the light chain variable domain (VL) and the heavy chain constant domain (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), VNAR fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin). CTLA-4 (Cytotoxic T Lymphocyte-associated Antigen 4) is a CD28-family receptor expressed on mainly CD4⁺ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details, see Journal of Immunological Methods 248 (1-2), 31-45 (2001). Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details, see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details, see Protein Eng. Des. Sel. 2004, 17, 455-462 and EP 1641818A1. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details, see Nature Biotechnology 23(12), 1556-1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details, see J. Biol. Chem 274, 24066-24073 (1999). Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details, see J. Mol. Biol. 332, 489-503 (2003), PNAS 100(4), 1700-1705 (2003) and J. Mol. Biol. 369, 1015-1028 (2007) and US20040132028A1. A single-domain antibody is an antibody fragment consisting of a single monomeric variable antibody domain. The first single domains were derived from the variable domain of the antibody heavy chain from camelids (nanobodies or VHH fragments). Furthermore, the term single-domain antibody includes an autonomous human heavy chain variable domain (aVH) or VNAR fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the beta-sandwich can be engineered to enable an Adnectin to specifically recognize a therapeutic target of interest. For further details, see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details, see Expert Opin. Biol. Ther. 5, 783-797 (2005). Microbodies are derived from naturally occurring microproteins of 25-50 amino acids in length which contain 3-4 cysteine bridges—examples of microproteins include KalataBl and conotoxin and knottins. The microproteins have a loop which can beengineered to include upto 25 amino acids without affecting the overall fold of the microprotein. For further details of engineered knottin domains, see WO2008098796.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

The term “antigen binding domain” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable domain (VL) and an antibody heavy chain variable domain (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g. from 10⁻⁹M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

A “tumor-associated antigen” or TAA as used herein refers to an antigenic determinant presented on the surface of a target cell, for example a cell in a tumor such as a cancer cell or a cell of the tumor stroma. In certain aspects, the target cell antigen is an antigen on the surface of a tumor cell. In one aspect, TAA is selected from the group consisting of Fibroblast Activation Protein (FAP), Carcinoembryonic Antigen (CEA), Folate receptor alpha (FolR1), Melanoma-associated Chondroitin Sulfate Proteoglycan (MCSP), Epidermal Growth Factor Receptor (EGFR), human epidermal growth factor receptor 2 (HER2) and p95HER2. In particular, the tumor-associated antigen is Fibroblast Activation Protein (FAP) or Carcinoembryonic Antigen (CEA). Further TAAs include HER3, EpCAM, TPBG (5T4), mesothelin, MUC1, and PSMA. TAAs also comprise B cell surface antigens such as CD19, CD20 and CD79b. Furthermore, the TAAs GPRC5D, BCMA and CD38 relating to Multiple Myeloma may also be included.

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP that results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO:2), or NCBI (www.ncbi.nlm.nih.gov/) RefSeq NP 004451.2. The extracellular domain (ECD) of human FAP extends from amino acid position 26 to 760. The amino acid sequence of a His-tagged human FAP ECD is shown in SEQ ID NO:135. The amino acid sequence of mouse FAP is shown in UniProt accession no. P97321 (version 126, SEQ ID NO:136), or NCBI RefSeq NP 032012.1. The extracellular domain (ECD) of mouse FAP extends from amino acid position 26 to 761. SEQ ID NO:137 shows the amino acid sequence of a His-tagged mouse FAP ECD. SEQ ID NO 138 shows the amino acid sequence, of a His-tagged cynomolgus FAP ECD. Preferably, an anti-FAP binding molecule of the invention binds to the extracellular domain of FAP.

The term “Carcinoembroynic antigen (CEA)”, also known as Carcinoembryonic antigen-related cell adhesion molecule 5 (CEACAM5), refers to any native CEA from any vertebrate source, including mammals such as primates (e.g. humans) non-human primates (e.g. cynomolgus monkeys) and rodents (e.g. mice and rats), unless otherwise indicated. The amino acid sequence of human CEA is shown in UniProt accession no. P06731 (version 151, SEQ ID NO:3). CEA has long been identified as a tumor-associated antigen (Gold and Freedman, J Exp Med., 121:439-462, 1965; Berinstein N. L., J Clin Oncol., 20:2197-2207, 2002). Originally classified as a protein expressed only in fetal tissue, CEA has now been identified in several normal adult tissues. These tissues are primarily epithelial in origin, including cells of the gastrointestinal, respiratory, and urogential tracts, and cells of colon, cervix, sweat glands, and prostate (Nap et al., Tumour Biol., 9(2-3):145-53, 1988; Nap et al., Cancer Res., 52(8):2329-23339, 1992). Tumors of epithelial origin, as well as their metastases, contain CEA as a tumor associated antigen. While the presence of CEA itself does not indicate transformation to a cancerous cell, the distribution of CEA is indicative. In normal tissue, CEA is generally expressed on the apical surface of the cell (Hammarstrom S., Semin Cancer Biol. 9(2):67-81 (1999)), making it inaccessible to antibody in the blood stream. In contrast to normal tissue, CEA tends to be expressed over the entire surface of cancerous cells (Hammarstrom S., Semin Cancer Biol. 9(2):67-81 (1999)). This change of expression pattern makes CEA accessible to antibody binding in cancerous cells. In addition, CEA expression increases in cancerous cells. Furthermore, increased CEA expression promotes increased intercellular adhesions, which may lead to metastasis (Marshall J., Semin Oncol., 30(a Suppl. 8):30-6, 2003). The prevalence of CEA expression in various tumor entities is generally very high. In concordance with published data, own analyses performed in tissue samples confirmed its high prevalence, with approximately 95% in colorectal carcinoma (CRC), 90% in pancreatic cancer, 80% in gastric cancer, 60% in non-small cell lung cancer (NSCLC, where it is co-expressed with HER3), and 40% in breast cancer; low expression was found in small cell lung cancer and glioblastoma.

CEA is readily cleaved from the cell surface and shed into the blood stream from tumors, either directly or via the lymphatics. Because of this property, the level of serum CEA has been used as a clinical marker for diagnosis of cancers and screening for recurrence of cancers, particularly colorectal cancer (Goldenberg D M., The International Journal of Biological Markers, 7:183-188, 1992; Chau I., et al., J Clin Oncol., 22:1420-1429, 2004; Flamini et al., Clin Cancer Res; 12(23):6985-6988, 2006).

The term “FolR1” refers to Folate receptor alpha and has been identified as a potential prognostic and therapeutic target in a number of cancers. It refers to any native FolR1 from any vertebrate source, including mammals such as primates (e.g. humans) non-human primates (e.g. cynomolgus monkeys) and rodents (e.g. mice and rats), unless otherwise indicated. The amino acid sequence of human FolR1 is shown in UniProt accession no. P15328 (SEQ ID NO: 139), murine FolR1 has the amino acid sequence of UniProt accession no. P35846 (SEQ ID NO:140) and cynomolgus FolR1 has the amino acid sequence as shown in UniProt accession no. G7PR14 (SEQ ID NO:141). FolR1 is an N-glycosylated protein expressed on plasma membrane of cells. FoIR1 has a high affinity for folic acid and for several reduced folic acid derivatives and mediates delivery of the physiological folate, 5-methyltetrahydrofolate, to the interior of cells. FOLR1 is a desirable target for FOLR1-directed cancer therapy as it is overexpressed in vast majority of ovarian cancers, as well as in many uterine, endometrial, pancreatic, renal, lung, and breast cancers, while the expression of FOLR1 on normal tissues is restricted to the apical membrane of epithelial cells in the kidney proximal tubules, alveolar pneumocytes of the lung, bladder, testes, choroid plexus, and thyroid. Recent studies have identified that FolR1 expression is particularly high in triple negative breast cancers (Necela et al. PloS One 2015, 10(3, e01271.33).

The term “Melanoma-associated Chondroitin Sulfate Proteoglycan (MCSP)”, also known as Chondroitin Sulfate Proteoglycan 4 (CSPG4) refers to any native MCSP from any vertebrate source, including mammals such as primates (e.g. humans) non-human primates (e.g. cynomolgus monkeys) and rodents (e.g. mice and rats), unless otherwise indicated. The amino acid sequence of human MCSP is shown in UniProt accession no. Q6UVK1 (version 103, SEQ ID NO:142). MCSP is a highly glycosylated integral membrane chondroitin sulfate proteoglycan consisting of an N-linked 280 kDa glycoprotein component and a 450-kDa chondroitin sulfate proteoglycan component expressed on the cell membrane (Ross et al., Arch. Biochem. Biophys. 1983, 225:370-38). MCSP is more broadly distributed in a number of normal and transformed cells. In particular, MCSP is found in almost all basal cells of the epidermis. MCSP is differentially expressed in melanoma cells, and was found to be expressed in more than 90% of benign nevi and melanoma lesions analyzed. MCSP has also been found to be expressed in tumors of nonmelanocytic origin, including basal cell carcinoma, various tumors of neural crest origin, and in breast carcinomas.

The term “Epidermal Growth Factor Receptor (EGFR)”, also named Proto-oncogene c-ErbB-1 or Receptor tyrosine-protein kinase erbB-1, refers to any native EGFR from any vertebrate source, including mammals such as primates (e.g. humans) non-human primates (e.g. cynomolgus monkeys) and rodents (e.g. mice and rats), unless otherwise indicated. The amino acid sequence of human EGFR is shown in UniProt accession no. P00533 (version 211, SEQ ID NO:143). The proto-oncogene “HER2”, (human epidermal growth factor receptor 2) encodes a protein tyrosine kinase (p185HER2) that is related to and somewhat homologous to the human epidermal growth factor receptor. HER2 is also known in the field as c-erbB-2, and sometimes by the name of the rat homolog, neu. Amplification and/or overexpression of HER2 is associated with multiple human malignancies and appears to be integrally involved in progression of 25-30% of human breast and ovarian cancers. Furthermore, the extent of amplification is inversely, correlated with the observed median patient survival time (Slamon, D. J. et al., Science 244:707-712 (1989)). The amino acid sequence of human HER2 is shown in UniProt accession no. P04626 (version 230, SEQ ID NO:144). The term “p95HER2” as used herein refers to a carboxy terminal fragment (CTF) of the HER2 receptor protein, which is also known as “611-CTF” or “100-115 kDa p95HER2”. The p95HER2 fragment is generated in the cell through initiation of translation of the HER2 mRNA at codon position 611 of the full-length HER2 molecule (Anido et al, EMBO J 25; 3234-44 (2006)). It has a molecular weight of 100 to 115 kDa and is expressed at the cell membrane, where it can form homodimers maintained by intermolecular disulfide bonds (Pedersen et al., Mol Cell Biol 29, 3319-31 (2009)). An exemplary sequence of human p95HER2 is given in SEQ ID NO: 145.

The term “CD28” (Cluster of differentiation 28, Tp44) refers to any CD28 protein from any vertebrate source, including mammals such as primates (e.g. humans) non-human primates (e.g. cynomolgus monkeys) and rodents (e.g. mice and rats), unless otherwise indicated. CD28 is expressed on T cells and provides co-stimulatory signals required for T cell activation and survival. T cell stimulation through CD28 in addition to the T-cell receptor (TCR) can provide a potent signal for the production of various interleukins. CD28 is the receptor for CD80 (B7.1) and CD86 (B7.2) proteins and is the only B7 receptor constitutively expressed on naive T cells. The amino acid sequence of human CD28 is shown in UniProt (www.uniprot.org) accession no. P10747 (SEQ ID NO:1).

An “agonistic antibody” refers to an antibody that comprises an agonistic function against a given receptor. In general, when an agonist ligand (factor) binds to a receptor, the tertiary structure of the receptor protein changes, and the receptor is activated (when the receptor is a. membrane protein, a cell growth signal or such is usually transducted). If the receptor is a dimer-forming type, an agonistic antibody can dimerize the receptor at an appropriate distance and angle, thus acting similarly to a ligand. An appropriate anti-receptor antibody can mimic dimerization of receptors performed by ligands, and thus can become an agonistic antibody,

A “CD28 agonistic antigen binding molecule” or “CD28 conventional agonistic antigen binding molecule” is an antigen binding molecule that mimicks CD28 natural ligands (CD80 or CD86) in their role to enhance T cell activation in presence of a T cell receptor signal (“signal 2”). A T cell needs two signals to become fully activated. Under physiological conditions “signal 1” arises form the interaction of T cell receptor (TCR) molecules with peptide/major histocompatibility complex (WIC) complexes on antigen presenting cells (APCs) and “signal 2” is provided by engagement of a costimulatory receptor, e.g. CD28. A CD28 agonistic antigen binding molecule is able to costimulate cells (signal 2). It is also able to induce T proliferation and cytokine secretion in combination with a molecule with specificity for the TCR complex, however the CD28 agonistic antigen binding molecule is not capable of fully activating T cells without additional stimulation of the TCR. There is however a subclass of CD28 specific antigen binding molecules, the so-called CD28 superagonistic antigen binding molecules. A “CD28 superagonistic antigen binding molecule” is a CD28 antigen binding molecule which is capable of fully activating T cells without additional stimulation of the TCR. A superagonist is normally defined as an agonist that is capable of producing a maximal response greater than the endogenous agonist (ligand) for the target receptor, and thus has an efficacy of more than 100%, however in relation to CD28, a CD28 superagonistic antigen binding molecule is meant to be a CD28 antigen binding molecule that is capable to induce T cell proliferation and cytokine secretion without prior cell activation (signal 1).

The term “variable domain” or “variable region” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR” as used herein refers to each of the regions of an antigen binding variable domain which are hypervariable in sequence and which determine antigen binding specificity, for example “complementarity determining regions” (“CDRs”). Generally, antigen binding domains comprise six CDRs: three in the VH (CDR-H1, CDR-H2, CDR-H3), and three in the VL (CDR-L1, CDR-L2, CDR-L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature. Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

As used herein, the term “affinity matured” in the context of antigen binding molecules (e.g., antibodies) refers to an antigen binding molecule that is derived from a reference antigen binding molecule, e.g., by mutation, binds to the same antigen, preferably binds to the same epitope, as the reference antibody; and has a higher affinity for the antigen than that of the reference antigen binding molecule. Affinity maturation generally involves modification of one or more amino acid residues in one or more CDRs of the antigen binding molecule. Typically, the affinity matured antigen binding molecule binds to the same epitope as the initial reference antigen binding molecule.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to Clq binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “Fc domain” or “Fc region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain. The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector functions” refers to those biological activities attributable to the Fc region of an antibody, which vary with the antibody isotype. Examples of antibody effector functions include: Clq binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 10³-fold and eliminated the human monocyte response to antibody-sensitized red blood cells (Armour, K. L., et al., Eur. J. Immunol. 29 (1999) 2613-2624).
FcγRII (CD32) binds complexed IgG with medium to low affinity and is widely expressed. This receptor can be divided into two sub-types, FcγRIIA and FcγRIIB FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, 5239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

The term “ADCC” or “antibody-dependent cellular cytotoxicity” is an immune mechanism leading to lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example, the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831). For example, the capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA) In particular, binding to FcγR on NK cells is measured.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcaRI (CD89). A particular activating Fc receptor is human FcγRIIIa (see UniProt accession no. P08637, version 141).

An “ectodomain” is the domain of a membrane protein that extends into the extracellular space (i.e. the space outside the target cell). Ectodomains are usually the parts of proteins that initiate contact with surfaces, which leads to signal transduction.

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 5, typically between 2 and 4, in particular 2, i.e. the peptides selected from the group consisting of GGGGS (SEQ ID NO:146) GGGGSGGGGS (SEQ ID NO:147), SGGGGSGGGG (SEQ ID NO:148) and GGGGSGGGGSGGGG (SEQ ID NO:149), but also include the sequences GSPGSSSSGS (SEQ ID NO:150), (G4S)₃ (SEQ ID NO:151), (G45)₄ (SEQ ID NO:152), GSGSGSGS (SEQ ID NO:153), GSGSGNGS (SEQ ID NO:154), GGSGSGSG (SEQ ID NO:155), GGSGSG (SEQ ID NO:156), GGSG (SEQ ID NO:157), GGSGNGSG (SEQ ID NO:158), GGNGSGSG (SEQ ID NO:159) and GGNGSG (SEQ ID NO:160). Peptide linkers of particular interest are (G4S) (SEQ ID NO:146), (G4S)₂ or GGGGSGGGGS (SEQ ID NO:147), (G4S)₃ (SEQ ID NO:151) and (G45)₄ (SEQ ID NO:152).

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

By “fused” or “connected” is meant that the components (e.g. a polypeptide and an ectodomain of said TNF ligand family member) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain embodiments, amino acid sequence variants of the CD28 antigen binding molecules provided herein are contemplated. For example, it may be desirable to improve the binding affinity and/or other biological properties of the CD28 antigen binding molecules. Amino acid sequence variants of the CD28 antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table B under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Tip (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more HVR residues are mutated and the variant antigen binding molecules displayed on phage and screened for a particular biological activity (e.g. binding affinity). In certain embodiments, substitutions, insertions, or deletions may occur within one or more HVRs so long as such alterations do not substantially reduce the ability of the antigen binding molecule to bind antigen. For example, conservative alterations (e.g., conservative substitutions as provided herein) that do not substantially reduce binding affinity may be made in HVRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of insertions include CD28 antigen binding molecules with a fusion to the N- or C-terminus to a polypeptide which increases the serum half-life of the CD28 antigen binding molecules.

In certain embodiments, the CD28 antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the agonistic ICOS-binding molecule comprises an Fc domain, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in agonistic ICOS-binding molecules may be made in order to create variants with certain improved properties. In one aspect, variants of agonistic ICOS-binding molecules are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). Further variants of the CD28 antigen binding molecules of the invention include those with bisected oligosaccharides, e.g., in which a biantennary oligosaccharide attached to the Fc region is bisected by GlcNAc. Such variants may have reduced fucosylation and/or improved ADCC function, see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain embodiments, it may be desirable to create cysteine engineered variants of the CD28 antigen binding molecules of the invention, e.g., “thioMAbs,” in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antigen binding molecules may be generated as described, e.g., in U.S. Pat. No. 7,521,541.

In certain aspects, the CD28 antigen binding molecules provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the bispecific antibody derivative will be used in a therapy under defined conditions, etc. In another aspect, conjugates of an antibody and non-proteinaceous moiety that may be selectively heated by exposure to radiation are provided. In one embodiment, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed. In another aspect, immunoconjugates of the CD28 antigen binding molecules provided herein maybe obtained. An “immunoconjugate” is an antibody conjugated to one or more heterologous molecule(s), including but not limited to a cytotoxic agent.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NSO cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” refers to or describes the physiological condition in mammals that is typically characterized by unregulated cell growth/proliferation. Thus, the term cancer as used herein refers to proliferative diseases, such as carcinoma, lymphomas (e.g., Hodgkin's and non-Hodgkin's lymphoma), blastoma, sarcoma, and leukemia. In particular, the term cancer includes lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers. In one aspect, the cancer is a solid tumor. In another aspect, the cancer is a haematological cancer, particularly leukemia, most particularly acute lymphoblastic leukemia (ALL) or acute myelogenous leukemia (AML).

It is understood that in all aspects and embodiments of the invention described the term “comprising” can also be replaced by “consisting of” and “consisting essentially of” aspects and embodiments.

Superagonistic CD28 antigen binding molecules of the invention

The invention provides novel superagonistic CD28 antigen binding molecules with particularly advantageous properties such as producibility, stability, binding affinity, biological activity, targeting efficiency, reduced toxicity, an extended dosage range that can be given to a patient and thereby a possibly enhanced efficacy. The novel superagonistic CD28 antigen binding molecules comprise an Fc domain composed of a first and a second subunit capable of stable association comprising one or more amino acid substitution that reduces the binding affinity of the antigen binding molecule to an Fc receptor and/or effector function (Fc silent) and thus unspecific cross-linking via Fc receptors is avoided. Instead, they comprise at least one antigen binding domain capable of specific binding to a tumor-associated antigen such as Fibroblast Activation Protein (FAP) or Carcinoembryonic Antigen (CEA) which causes cross-linking at the tumor site. Thus, tumor-specific T cell activation is achieved.

Herein provided is a superagonistic CD28 antigen binding molecule, which is capable of multivalent binding to CD28 and comprises

(a) two or more antigen binding domains capable of specific binding to CD28,
(b) at least one antigen binding domain capable of specific binding to a tumor-associated antigen, and
(c) an Fc domain composed of a first and a second subunit capable of stable association comprising one or more amino acid substitution that reduces the binding affinity of the antigen binding molecule to an Fc receptor and/or effector function.

In one particular aspect, the superagonistic CD28 antigen binding molecule is capable of bivalent binding to CD28 and comprises two antigen binding domains capable of specific binding to CD28.

In one aspect, a superagonistic CD28 antigen binding molecule as defined herein before is provided, wherein the Fc domain is an IgG, particularly an IgG1 Fc domain or an IgG4 Fc domain. In one particular aspect, the Fc domain composed of a first and a second subunit capable of stable association is an IgG1 Fc domain. The Fc domain comprises one or more amino acid substitution that reduces the binding affinity of the antigen binding molecule to an Fc receptor and/or reduces or abolishes effector function. In one aspect, the Fc domain comprises the amino acid substitutions L234A and L235A (numbering according to Kabat EU index). In one aspect, the Fc domain is of human IgG1 subclass and comprises the amino acid mutations L234A, L235A and P329G (numbering according to Kabat EU index).

In one aspect, provided is a superagonistic CD28 antigen binding molecule as defined herein before, wherein each of the antigen binding domains capable of specific binding to CD28 comprises

(i) a heavy chain variable region (V_{H}CD28) comprising a heavy chain complementary determining region CDR-H1 of SEQ ID NO: 20, a CDR-H2 of SEQ ID NO: 21, and a CDR-H3 of SEQ ID NO: 22, and a light chain variable region (V_{L}CD28) comprising a light chain complementary determining region CDR-L1 of SEQ ID NO: 23, a CDR-L2 of SEQ ID NO: 24 and a CDR-L3 of SEQ ID NO: 25; or
(ii) a heavy chain variable region (V_{H}CD28) comprising a CDR-H1 of SEQ ID NO: 36, a CDR-H2 of SEQ ID NO: 37, and a CDR-H3 of SEQ ID NO: 38, and a light chain variable region (V_{L}CD28) comprising a CDR-L1 of SEQ ID NO: 39, a CDR-L2 of SEQ ID NO: 40 and a CDR-L3 of SEQ ID NO: 41.

In one aspect, each of the antigen binding domains capable of specific binding to CD28 of the superagonistic CD28 antigen binding molecule comprises a heavy chain variable region (V_{H}CD28) comprising a CDR-H1 of SEQ ID NO: 36, a CDR-H2 of SEQ ID NO: 37, and a CDR-H3 of SEQ ID NO: 38, and a light chain variable region (V_{L}CD28) comprising a CDR-L1 of SEQ ID NO: 39, a CDR-L2 of SEQ ID NO: 40 and a CDR-L3 of SEQ ID NO: 41.

In another aspect, each of the antigen binding domains capable of specific binding to CD28 of the superagonistic CD28 antigen binding molecule comprises a heavy chain variable region (V_{H}CD28) comprising a CDR-H1 of SEQ ID NO: 20, a CDR-H2 of SEQ ID NO: 21, and a CDR-H3 of SEQ ID NO: 22, and a light chain variable region (V_{L}CD28) comprising a CDR-L1 of SEQ ID NO: 23, a CDR-L2 of SEQ ID NO: 24 and a CDR-L3 of SEQ ID NO: 25.

Furthermore, provided is a superagonistic CD28 antigen binding molecule as defined herein before, wherein each of the antigen binding domains capable of specific binding to CD28 comprises a heavy chain variable region (V_{H}CD28) comprising an amino acid sequence that is at least about 95%, 96%, 97%, 98%, 99% or 100% identical to the amino acid sequence of SEQ ID NO:26, and a light chain variable region (V_{L}CD28) comprising an amino acid sequence that is at least about 95%, 96%, 97%, 98%, 99% or 100% identical to the amino acid sequence of SEQ ID NO:27.

In a further aspect, a superagonistic CD28 antigen binding molecule is provided, wherein each of the antigen binding domains capable of specific binding to CD28 comprises a heavy chain variable region (V_{H}CD28) comprising an amino acid sequence selected from the group consisting of SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50 and SEQ ID NO:51, and a light chain variable region (V_{L}CD28) comprising an amino acid sequence selected from the group consisting of SEQ ID NO:27, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60 and SEQ ID NO:61.

In another aspect, provided is a superagonistic CD28 antigen binding molecule, wherein each of the antigen binding domains capable of specific binding to CD28 comprises

(a) a heavy chain variable region (V_{H}CD28) comprising the amino acid sequence of SEQ ID NO:47 and a light chain variable region (V_{L}CD28) comprising the amino acid sequence of SEQ ID NO:54, or

(b) a heavy chain variable region (V_{H}CD28) comprising the amino acid sequence of SEQ ID NO:47 and a light chain variable region (V_{L}CD28) comprising the amino acid sequence of SEQ ID NO:27, or

(c) a heavy chain variable region (V_{H}CD28) comprising the amino acid sequence of SEQ ID NO:51 and a light chain variable region (V_{L}CD28) comprising the amino acid sequence of SEQ ID NO:61, or

(d) a heavy chain variable region (V_{H}CD28) comprising the amino acid sequence of SEQ ID NO:46 and a light chain variable region (V_{L}CD28) comprising the amino acid sequence of SEQ ID NO:53, or

(e) a heavy chain variable region (V_{H}CD28) comprising the amino acid sequence of SEQ ID NO:46 and a light chain variable region (V_{L}CD28) comprising the amino acid sequence of SEQ ID NO:54, or

(f) a heavy chain variable region (V_{H}CD28) comprising the amino acid sequence of SEQ ID NO:46 and a light chain variable region (V_{L}CD28) comprising the amino acid sequence of SEQ ID NO:59, or

(g) a heavy chain variable region (V_{H}CD28) comprising the amino acid sequence of SEQ ID NO:46 and a light chain variable region (V_{L}CD28) comprising the amino acid sequence of SEQ ID NO:27, or

(h) a heavy chain variable region (V_{H}CD28) comprising the amino acid sequence of SEQ ID NO:43 and a light chain variable region (V_{L}CD28) comprising the amino acid sequence of SEQ ID NO:27, or

(i) a heavy chain variable region (V_{H}CD28) comprising the amino acid sequence of SEQ ID NO:42 and a light chain variable region (V_{L}CD28) comprising the amino acid sequence of SEQ ID NO:53, or

(j) a heavy chain variable region (V_{H}CD28) comprising the amino acid sequence of SEQ ID NO:42 and a light chain variable region (V_{L}CD28) comprising the amino acid sequence of SEQ ID NO:59, or

(k) a heavy chain variable region (V_{H}CD28) comprising the amino acid sequence of SEQ ID NO:42 and a light chain variable region (V_{L}CD28) comprising the amino acid sequence of SEQ ID NO:27.

In one particular aspect, a superagonistic CD28 antigen binding molecule is provided, wherein each of the antigen binding domains capable of specific binding to CD28 comprises a heavy chain variable region (V_{H}CD28) comprising the amino acid sequence of SEQ ID NO:47 and a light chain variable region (V_{L}CD28) comprising the amino acid sequence of SEQ ID NO:54.

In another particular aspect, a superagonistic CD28 antigen binding molecule is provided, wherein each of the antigen binding domains capable of specific binding to CD28 comprises a heavy chain variable region (V_{H}CD28) comprising the amino acid sequence of SEQ ID NO:46 and a light chain variable region (V_{L}CD28) comprising the amino acid sequence of SEQ ID NO:53.

In further particular aspect, a superagonistic CD28 antigen binding molecule is provided, wherein each of the antigen binding domains capable of specific binding to CD28 comprises a heavy chain variable region (V_{H}CD28) comprising the amino acid sequence of SEQ ID NO:42 and a light chain variable region (V_{L}CD28) comprising the amino acid sequence of SEQ ID NO:27.

In a further aspect, provided is a superagonistic CD28 antigen binding molecule as defined herein before, wherein each of the antigen binding domains capable of specific binding to CD28 is a Fab fragment.

In one aspect, a superagonistic CD28 antigen binding molecule is provided, wherein the antigen binding domain capable of specific binding to a tumor-associated antigen is an antigen binding domain capable of specific binding to Carcinoembryonic Antigen (CEA).

In one aspect, provided is a superagonistic CD28 antigen binding molecule as described herein, wherein the antigen binding domain capable of specific binding to CEA comprises a heavy chain variable region (V_{H}CEA) comprising (i) CDR-H1 comprising the amino acid sequence of SEQ ID NO:127, (ii) CDR-H2 comprising the amino acid sequence of SEQ ID NO:128, and (iii) CDR-H3 comprising the amino acid sequence of SEQ ID NO:129, and a light chain variable region (V_{L}CEA) comprising (iv) CDR-L1 comprising the amino acid sequence of SEQ ID NO:130, (v) CDR-L2 comprising the amino acid sequence of SEQ ID NO:131, and (vi) CDR-L3 comprising the amino acid sequence of SEQ ID NO:132. Particularly, the antigen binding domain capable of specific binding to CEA comprises a heavy chain variable region

(V_{H}CEA) comprising an amino acid sequence that is at least about 95%, 96%, 97%, 98%, 99% or 100% identical to the amino acid sequence of SEQ ID NO:133, and a light chain variable region (V_{L}CEA) comprising an amino acid sequence that is at least about 95%, 96%, 97%, 98%, 99% or 100% identical to the amino acid sequence of SEQ ID NO:134.

In another aspect, a superagonistic CD28 antigen binding molecule is provided, wherein the antigen binding domain capable of specific binding to a tumor-associated antigen is an antigen binding domain capable of specific binding to Fibroblast Activation Protein (FAP).

In one aspect, provided is a superagonistic CD28 antigen binding molecule as described herein, wherein the antigen binding domain capable of specific binding to FAP comprises

(a) a heavy chain variable region (V_{H}FAP) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}FAP) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17, or

(b) a heavy chain variable region (V_{H}FAP) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}FAP) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9. In particular, the antigen binding domain capable of specific binding to FAP comprises a heavy chain variable region (V_{H}FAP) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}FAP) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17.

In one aspect, a superagonistic CD28 antigen binding molecule is provided, wherein the antigen binding domain capable of specific binding to FAP comprises (a) a heavy chain variable region (V_{H}FAP) comprising an amino acid sequence that is at least about 95%, 96%, 97%, 98%, 99% or 100% identical to the amino acid sequence of SEQ ID NO:18, and a light chain variable region (V_{L}FAP) comprising an amino acid sequence that is at least about 95%, 96%, 97%, 98%, 99% or 100% identical to the amino acid sequence of SEQ ID NO:19, or (b) a heavy chain variable region (V_{H}FAP) comprising an amino acid sequence that is at least about 95%, 96%, 97%, 98%, 99% or 100% identical to the amino acid sequence of SEQ ID NO:10, and a light chain variable region (V_{L}FAP) comprising an amino acid sequence that is at least about 95%, 96%, 97%, 98%, 99% or 100% identical to the amino acid sequence of SEQ ID NO:11. Particularly, the antigen binding domain capable of specific binding to FAP comprises a heavy chain variable region (V_{H}FAP) comprising the amino acid sequence of SEQ ID NO:18 and a light chain variable region (V_{L}FAP) comprising the amino acid sequence of SEQ ID NO:19.

Superagonistic CD28 antigen binding molecules bivalent for binding to CD28 and monovalent for binding to the tumor-associated antigen (1+2 format)

In another aspect, provided is a superagonistic CD28 antigen binding molecule as described herein, comprising

(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and

(b) a VH and VL domain capable of specific binding to a tumor-associated antigen, wherein the VH domain is connected via a peptide linker to the C-terminus of one of the two heavy chains and wherein the VL domain is connected via a peptide linker to the C-terminus of the second heavy chain.

In one aspect, the peptide linker comprises an amino acid sequence selected from SEQ ID NO:146, SEQ ID NO:147, SEQ ID NO:151 and SEQ ID NO:152. More particularly, the peptide linker comprises the SEQ ID NO:152.

In another aspect, the superagonistic CD28 antigen binding molecule comprises

(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and
(b) a VH and VL domain capable of specific binding to a tumor-associated antigen, wherein the VH domain is connected via a peptide linker to the C-terminus of the Fc knob heavy chain and wherein the VL domain is connected via a peptide linker to the C-terminus of the Fc hole heavy chain.

In a particular aspect, provided is a superagonistic CD28 antigen binding molecule comprising two light chains, each comprising the amino acid sequence of SEQ ID NO:62, a first heavy chain comprising the amino acid sequence of SEQ ID NO:71, and a second heavy chain comprising the amino acid sequence of SEQ ID NO:72.

In another particular aspect, provided is a superagonistic CD28 antigen binding molecule comprising two light chains, each comprising the amino acid sequence of SEQ ID NO:62, a first heavy chain comprising the amino acid sequence of SEQ ID NO:83, and a second heavy chain comprising the amino acid sequence of SEQ ID NO:84.

In a further aspect, the superagonistic CD28 antigen binding molecule comprises

(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and
(b) a VH and VL domain capable of specific binding to a tumor-associated antigen, wherein the VH domain is connected via a peptide linker to the C-terminus of the Fc hole heavy chain and wherein the VL domain is connected via a peptide linker to the C-terminus of the Fc knob heavy chain.

In a further aspect, provided is a superagonistic CD28 antigen binding molecule as described herein, comprising

(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and

(b) a crossFab fragment capable of specific binding to a tumor-associated antigen which is connected via a peptide linker to the C-terminus of one of the two heavy chains.

Superagonistic CD28 Antigen Binding Molecules Bivalent for Binding to CD28 and Bivalent for Binding to the Tumor-Associated Antigen (2+2 Format)

In another aspect, a superagonistic CD28 antigen binding molecule as disclosed herein is provided, comprising

(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and

(b) two crossFab fragments capable of specific binding to a tumor-associated antigen, wherein one crossFab fragment is connected via a peptide linker to the C-terminus of one of the two heavy chains and wherein the other crossFab fragment is connected via a peptide linker to the C-terminus of the second heavy chain.

In one aspect, the superagonistic CD28 antigen binding molecule as described herein before comprises two crossFab fragments capable of specific binding to a tumor-associated antigen, wherein one crossFab fragment is connected via a peptide linker to the C-terminus of one of the two heavy chains and wherein the other crossFab fragment is connected via a peptide linker to the C-terminus of the second heavy chain, and wherein the CH1 and CL domains are exchanged in the crossFabs fragments. In a further aspect, the crossFab fragments are each fused at the N-terminus of the VH domain to the C-terminus of Fc domain.

In a particular aspect, provided is a superagonistic CD28 antigen binding molecule comprising two light chains, each comprising the amino acid sequence of SEQ ID NO:65, two light chains, each comprising the amino acid sequence of SEQ ID NO:74, and two heavy chains, each comprising the amino acid sequence of SEQ ID NO:73.

In another particular aspect, provided is a superagonistic CD28 antigen binding molecule comprising two light chains, each comprising the amino acid sequence of SEQ ID NO:65, two light chains, each comprising the amino acid sequence of SEQ ID NO:82, and two heavy chains, each comprising the amino acid sequence of SEQ ID NO:81.

Trispecific Superagonistic CD28 Antigen Binding Molecules Bivalent for Binding to CD28, Monovalent for Binding to FAP and Monovalent for Binding to CEA

In another aspect, provided is a superagonistic CD28 antigen binding molecule as described herein, comprising

(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function,

(b) a VH and VL domain capable of specific binding to FAP, wherein the VH domain is connected via a peptide linker to the C-terminus of one of the two heavy chains and wherein the VL domain is connected via a peptide linker to the C-terminus of the second heavy chain, and

(c) a crossFab fragment capable of specific binding to CEA which is connected via a peptide linker to the C-terminus of the VH domain capable of specific binding to FAP.

In a particular aspect, provided is a superagonistic CD28 antigen binding molecule comprising two light chains, each comprising the amino acid sequence of SEQ ID NO:62, a light chain comprising the amino acid sequence of SEQ ID NO:109, a first heavy chain comprising the amino acid sequence of SEQ ID NO:107, and a second heavy chain comprising the amino acid sequence of SEQ ID NO:108.

In another aspect, provided is a superagonistic CD28 antigen binding molecule as described herein, comprising

(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function,

(b) a VH and VL domain capable of specific binding to FAP, wherein the VH domain is connected via a peptide linker to the C-terminus of one of the two heavy chains and wherein the VL domain is connected via a peptide linker to the C-terminus of the second heavy chain, and

(c) a VH and VL domain capable of specific binding to CEA, wherein the VH domain is connected via a peptide linker to the C-terminus of the VH domain capable of specific binding to FAP and wherein the VL domain is connected via a peptide linker to the C-terminus of the VL domain capable of specific binding to FAP.

In a particular aspect, provided is a superagonistic CD28 antigen binding molecule comprising two light chains, each comprising the amino acid sequence of SEQ ID NO:62, a first heavy chain comprising the amino acid sequence of SEQ ID NO:110, and a second heavy chain comprising the amino acid sequence of SEQ ID NO:111.

Fc Domain Modifications Reducing Fc Receptor Binding and/or Effector Function

The Fc domain of the superagonistic CD28 antigen binding molecule of the invention consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. The Fc domain confers favorable pharmacokinetic properties to the antigen binding molecules of the invention, including a long serum half-life which contributes to good accumulation in the target tissue and a favorable tissue-blood distribution ratio. On the other side, it may, however, lead to undesirable targeting of the bispecific antibodies of the invention to cells expressing Fc receptors rather than to the preferred antigen-bearing cells.

Accordingly, the Fc domain of the superagonistic CD28 antigen binding molecule of the invention exhibits reduced binding affinity to an Fc receptor and/or reduced effector function, as compared to a native IgG1 Fc domain. In one aspect, the Fc does not substantially bind to an Fc receptor and/or does not induce effector function. In a particular aspect the Fc receptor is an Fcγ receptor. In one aspect, the Fc receptor is a human Fc receptor. In a specific aspect, the Fc receptor is an activating human Fcγ receptor, more specifically human FcγRIIIa, FcγRI or FcγRIIa, most specifically human FcγRIIIa. In one aspect, the Fc domain does not induce effector function. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

In certain aspects, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In one particular aspect, the invention provides an antibody, wherein the Fc region comprises one or more amino acid substitution that reduces binding to an Fc receptor, in particular towards Fcγ receptor. In one aspect, the invention provides an antibody, wherein the Fc region comprises one or more amino acid substitution and wherein the ADCC induced by the antibody is reduced to 0-20% of the ADCC induced by an antibody comprising the wild-type human IgG1 Fc region.

In one aspect, the Fc domain of the antibody of the invention comprises one or more amino acid mutation that reduces the binding affinity of the Fc domain to an Fc receptor and/or effector function. Typically, the same one or more amino acid mutation is present in each of the two subunits of the Fc domain. In particular, the Fc domain comprises an amino acid substitution at a position of E233, L234, L235, N297, P331 and P329 (EU numbering). In particular, the Fc domain comprises amino acid substitutions at positions 234 and 235 (EU numbering) and/or 329 (EU numbering) of the IgG heavy chains. More particularly, provided is an antibody according to the invention which comprises an Fc domain with the amino acid substitutions L234A, L235A and P329G (“P329G LALA”, EU numbering) in the IgG heavy chains. The amino acid substitutions L234A and L235A refer to the so-called LALA mutation. The “P329G LALA” combination of amino acid substitutions almost completely abolishes Fcγ receptor binding of a human IgG1 Fc domain and is described in International Patent Appl. Publ. No. WO 2012/130831 A1 which also describes methods of preparing such mutant Fc domains and methods for determining its properties such as Fc receptor binding or effector functions.

Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In another aspect, the Fc domain is an IgG4 Fc domain. IgG4 antibodies exhibit reduced binding affinity to Fc receptors and reduced effector functions as compared to IgG1 antibodies. In a more specific aspect, the Fc domain is an IgG4 Fc domain comprising an amino acid substitution at position 5228 (Kabat numbering), particularly the amino acid substitution S228P. In a more specific aspect, the Fc domain is an IgG4 Fc domain comprising amino acid substitutions L235E and S228P and P329G (EU numbering). Such IgG4 Fc domain mutants and their Fcγ receptor binding properties are also described in WO 2012/130831.

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. Alternatively, binding affinity of Fc domains or cell activating antibodies comprising an Fc domain for Fc receptors may be evaluated using cell lines known to express particular Fc receptors, such as human NK cells expressing FcγIIIa receptor.

Effector function of an Fc domain, or antigen binding molecules of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in an animal model such as that disclosed in Clynes et al., Proc Natl Acad Sci USA 95, 652-656 (1998).

In some aspects, binding of the Fc domain to a complement component, specifically to C1q, is reduced. Accordingly, in some aspects wherein the Fc domain is engineered to have reduced effector function, said reduced effector function includes reduced CDC. C1q binding assays may be carried out to determine whether the bispecific antibodies of the invention are able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

In one particular aspect, the Fc domain exhibiting reduced binding affinity to an Fc receptor and/or reduced effector function, as compared to a native IgG1 Fc domain, is a human IgG1 Fc domain comprising the amino acid substitutions L234A, L235A and optionally P329G, or a human IgG4 Fc domain comprising the amino acid substitutions S228P, L235E and optionally P329G (numberings according to Kabat EU index). More particularly, it is a human IgG1 Fc domain comprising the amino acid substitutions L234A, L235A and P329G (numbering according to Kabat EU index).

Fc Domain Modifications Promoting Heterodimerization

The superagonistic CD28 antigen binding molecules of the invention comprise different antigen-binding sites, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the bispecific antigen binding molecules of the invention in recombinant production, it will thus be advantageous to introduce in the Fc domain of the bispecific antigen binding molecules of the invention a modification promoting the association of the desired polypeptides.

Accordingly, in particular aspects the invention relates to the superagonistic CD28 antigen binding molecule comprising (a) two or more antigen binding domains capable of specific binding to CD28, (b) at least one antigen binding domain capable of specific binding to a tumor-associated antigen, and (c) an Fc domain composed of a first and a second subunit capable of stable association comprising one or more amino acid substitution that reduces the binding affinity of the antigen binding molecule to an Fc receptor and/or effector function, wherein the Fc domain comprises a modification promoting the association of the first and second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

In a specific aspect said modification is a so-called “knob-into-hole” modification, comprising a “knob” modification in one of the two subunits of the Fc domain and a “hole” modification in the other one of the two subunits of the Fc domain. Thus, the invention relates to the superagonistic CD28 antigen binding molecule comprising (a) two or more antigen binding domains capable of specific binding to CD28, (b) at least one antigen binding domain capable of specific binding to a tumor-associated antigen, and (c) an Fc domain composed of a first and a second subunit capable of stable association comprising one or more amino acid substitution that reduces the binding affinity of the antigen binding molecule to an Fc receptor and/or effector function, wherein the first subunit of the Fc domain comprises knobs and the second subunit of the Fc domain comprises holes according to the knobs into holes method. In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in one aspect, in the CH3 domain of the first subunit of the Fc domain of the bispecific antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. The protuberance and cavity can be made by altering the nucleic acid encoding the polypeptides, e.g. by site-specific mutagenesis, or by peptide synthesis. In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A).

In yet a further aspect, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter (2001), J Immunol Methods 248, 7-15). In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

The C-terminus of the heavy chain of the superagonistic CD28 antigen binding molecule as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, a CD28 antigen binding molecule comprising a heavy chain including a C-terminal CH3 domain as specified herein, comprises the C-terminal glycine-lysine dipeptide (G446 and K447, numbering according to Kabat EU index). In one aspect of all aspects as reported herein, a CD28 antigen binding molecule comprising a heavy chain including a C-terminal CH3 domain, as specified herein, comprises a C-terminal glycine residue (G446, numbering according to Kabat EU index).

Modifications in the Fab Domains

In one aspect, the invention relates to a superagonistic CD28 antigen binding molecule comprising (a) two or more antigen binding domains capable of specific binding to CD28, (b) at least one antigen binding domain capable of specific binding to a tumor-associated antigen, and (c) an Fc domain composed of a first and a second subunit capable of stable association comprising one or more amino acid substitution that reduces the binding affinity of the antigen binding molecule to an Fc receptor and/or effector function, wherein the at least one antigen binding domain capable of specific binding to a tumor-associated antigen is a Fab fragment and in the Fab fragment either the variable domains VH and VL or the constant domains CH1 and CL are exchanged according to the Crossmab technology.

Multispecific antibodies with a domain replacement/exchange in one binding arm (CrossMabVH-VL or CrossMabCH-CL) are described in detail in WO2009/080252 and Schaefer, W. et al, PNAS, 108 (2011) 11187-1191. They clearly reduce the byproducts caused by the mismatch of a light chain against a first antigen with the wrong heavy chain against the second antigen (compared to approaches without such domain exchange).

In one aspect, the invention relates to a superagonistic CD28 antigen binding molecule comprising (a) two or more antigen binding domains capable of specific binding to CD28, (b) two antigen binding domains capable of specific binding to a tumor-associated antigen, and (c) an Fc domain composed of a first and a second subunit capable of stable association comprising one or more amino acid substitution that reduces the binding affinity of the antigen binding molecule to an Fc receptor and/or effector function, wherein in the Fab fragments capable of specific binding to a tumor-associated antigen the constant domains CL and CH1 are replaced by each other so that the CH1 domain is part of the light chain and the CL domain is part of the heavy chain.

In another aspect, and to further improve correct pairing, the superagonistic CD28 antigen binding molecule comprising (a) two or more antigen binding domains capable of specific binding to CD28, (b) two antigen binding domains capable of specific binding to a tumor-associated antigen, and (c) an Fc domain composed of a first and a second subunit capable of stable association comprising one or more amino acid substitution that reduces the binding affinity of the antigen binding molecule to an Fc receptor and/or effector function, can contain different charged amino acid substitutions (so-called “charged residues”). These modifications are introduced in the crossed or non-crossed CH1 and CL domains. In a particular aspect, the invention relates to a superagonistic CD28 antigen binding molecule, wherein in one of CL domains the amino acid at position 123 (EU numbering) has been replaced by arginine (R) and the amino acid at position 124 (EU numbering) has been substituted by lysine (K) and wherein in one of the CH1 domains the amino acids at position 147 (EU numbering) and at position 213 (EU numbering) have been substituted by glutamic acid (E). In one particular aspect, in the CL domain of the Fab fragment capable of specific binding to CD28 the amino acid at position 123 (EU numbering) has been replaced by arginine (R) and the amino acid at position 124 (EU numbering) has been substituted by lysine (K) and in the CH1 domain of the Fab fragment capable of specific binding to CD28 the amino acids at position 147 (EU numbering) and at position 213 (EU numbering) have been substituted by glutamic acid (E).

Polynucleotides

The invention further provides isolated polynucleotides encoding a superagonistic CD28 antigen binding molecule as described herein or a fragment thereof.

The isolated polynucleotides encoding the superagonistic CD28 antigen binding molecule of the invention may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated polynucleotide encodes the entire superagonistic CD28 antigen binding molecule according to the invention as described herein. In other aspects, the isolated polynucleotide encodes a polypeptide comprised in the superagonistic CD28 antigen binding molecule according to the invention as described herein.

In certain aspects the polynucleotide or nucleic acid is DNA. In other aspects, a polynucleotide of the present invention is RNA, for example, in the form of messenger RNA (mRNA). RNA of the present invention may be single stranded or double stranded.

Recombinant Methods

Superagonistic CD28 antigen binding molecules of the invention may be obtained, for example, by solid-state peptide synthesis (e.g. Merrifield solid phase synthesis) or recombinant production. For recombinant production one or more polynucleotide encoding the superagonistic CD28 antigen binding molecule or polypeptide fragments thereof, e.g., as described above, is isolated and inserted into one or more vectors for further cloning and/or expression in a host cell. Such polynucleotide may be readily isolated and sequenced using conventional procedures. In one aspect of the invention, a vector, preferably an expression vector, comprising one or more of the polynucleotides of the invention is provided. Methods which are well known to those skilled in the art can be used to construct expression vectors containing the coding sequence of the antibody (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the antibody or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the antibody of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit d-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the antibody or polypeptide fragments thereof is desired, DNA encoding a signal sequence may be placed upstream of the nucleic acid an antibody of the invention or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the superagonistic CD28 antigen binding molecule may be included within or at the ends of the polynucleotide encoding an antibody of the invention or polypeptide fragments thereof.

In a further aspect of the invention, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) an antibody of the invention of the invention. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr-CHO cells (Urlaub et al., Proc Natl Acad Sci USA 77, 4216 (1980)); and myeloma cell lines such as YO, NSO, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., YO, NSO, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain.

In one aspect, a method of producing a superagonistic CD28 antigen binding molecule of the invention or polypeptide fragments thereof is provided, wherein the method comprises culturing a host cell comprising polynucleotides encoding the antibody of the invention or polypeptide fragments thereof, as provided herein, under conditions suitable for expression of the antibody of the invention or polypeptide fragments thereof, and recovering the antibody of the invention or polypeptide fragments thereof from the host cell (or host cell culture medium).

In certain embodiments the moieties capable of specific binding to a target cell antigen (e.g. Fab fragments) forming part of the antigen binding molecule comprise at least an immunoglobulin variable region capable of binding to an antigen. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of immunoglobulin can be used in the invention. Non-limiting immunoglobulins useful in the present invention can be of murine, primate, or human origin. If the fusion protein is intended for human use, a chimeric form of immunoglobulin may be used wherein the constant regions of the immunoglobulin are from a human. A humanized or fully human form of the immunoglobulin can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Particular immunoglobulins according to the invention are human immunoglobulins. Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N.J., 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain aspects, the superagonistic CD28 antigen binding molecules are engineered to have enhanced binding affinity according to, for example, the methods disclosed in PCT publication WO 2012/020006 (see Examples relating to affinity maturation) or U.S. Pat. Appl. Publ. No. 2004/0132066. The ability of the antigen binding molecules of the invention to bind to a specific antigenic determinant can be measured either through an enzyme-linked immunosorbent assay (ELISA) or other techniques familiar to one of skill in the art, e.g. surface plasmon resonance technique (Liljeblad, et al., Glyco J 17, 323-329 (2000)), and traditional binding assays (Heeley, Endocr Res 28, 217-229 (2002)). Competition assays may be used to identify an antigen binding molecule that competes with a reference antibody for binding to a particular antigen. In certain embodiments, such a competing antigen binding molecule binds to the same epitope (e.g. a linear or a conformational epitope) that is bound by the reference antigen binding molecule. Detailed exemplary methods for mapping an epitope to which an antigen binding molecule binds are provided in Morris (1996) “Epitope Mapping Protocols”, in Methods in Molecular Biology vol. 66 (Humana Press, Totowa, N.J.). In an exemplary competition assay, immobilized antigen is incubated in a solution comprising a first labeled antigen binding molecule that binds to the antigen and a second unlabeled antigen binding molecule that is being tested for its ability to compete with the first antigen binding molecule for binding to the antigen. The second antigen binding molecule may be present in a hybridoma supernatant. As a control, immobilized antigen is incubated in a solution comprising the first labeled antigen binding molecule but not the second unlabeled antigen binding molecule. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antigen binding molecule is competing with the first antigen binding molecule for binding to the antigen. See Harlow and Lane (1988) Antibodies: A Laboratory Manual ch.14 (Cold Spring Harbor Laboratory, Cold Spring Harbor, N.Y.).

Superagonistic CD28 antigen binding molecules of the invention prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the antigen binding molecule binds. For example, for affinity chromatography purification of antigen binding molecules of the invention, a matrix with protein A or protein G may be used. Sequential Protein A or G affinity chromatography and size exclusion chromatography can be used to isolate an antigen binding molecule essentially as described in the Examples. The purity of the CD28 antigen binding molecule or fragments thereof can be determined by any of a variety of well-known analytical methods including gel electrophoresis, high pressure liquid chromatography, and the like. For example, the CD28 antigen binding molecule expressed as described in the Examples were shown to be intact and properly assembled as demonstrated by reducing and non-reducing SDS-PAGE.

Assays

The superagonistic CD28 antigen binding molecules provided herein may be identified, screened for, or characterized for their physical/chemical properties and/or biological activities by various assays known in the art.

1. Affinity Assays

The affinity of the antigen binding molecule provided herein for the corresponding target can be determined in accordance with the methods set forth in the Examples by surface plasmon resonance (SPR), using standard instrumentation such as a Proteon instrument (Bio-rad), and receptors or target proteins such as may be obtained by recombinant expression. The affinity of the TNF family ligand trimer-containing antigen binding molecule for the target cell antigen can also be determined by surface plasmon resonance (SPR), using standard instrumentation such as a Proteon instrument (Bio-rad), and receptors or target proteins such as may be obtained by recombinant expression. A specific illustrative and exemplary embodiment for measuring binding affinity is described in Example 4. According to one aspect, K_{D} is measured by surface plasmon resonance using a Proteon® machine (Bio-Rad) at 25° C.

2. Binding Assays and Other Assays

Binding of the bispecific antigen binding molecule provided herein to the corresponding receptor expressing cells may be evaluated using cell lines expressing the particular receptor or target antigen, for example by flow cytometry (FACS) or by surface plasmon resonance (SPR). In one aspect, CHO cells expressing human CD28 (parental cell line CHO-k1 ATCC # CCL-61, modified to stably overexpress human CD28) are used in the binding assay.

In a further aspect, cancer cell lines expressing the target cell antigen, for example FAP or CEA, were used to demonstrate the binding of the bispecific antigen binding molecules to the target cell antigen.

3. Activity Assays

In one aspect, assays are provided for identifying CD28 antigen binding molecules having biological activity. Biological activity may include, e.g. T cell proliferation and cytokine secretion as measured with the method as described in Example 5 or tumor cell killing as measured in Example 6. Antibodies having such biological activity in vivo and/or in vitro are also provided.

Pharmaceutical Compositions, Formulations and Routes of Administration

In a further aspect, the invention provides pharmaceutical compositions comprising any of the superagonistic CD28 antigen binding molecules provided herein, e.g., for use in any of the below therapeutic methods. In one embodiment, a pharmaceutical composition comprises a superagonistic CD28 antigen binding molecule provided herein and at least one pharmaceutically acceptable excipient. In another embodiment, a pharmaceutical composition comprises an superagonistic CD28 antigen binding molecule provided herein and at least one additional therapeutic agent, e.g., as described below.

Pharmaceutical compositions of the present invention comprise a therapeutically effective amount of one or more antigen binding molecules dissolved or dispersed in a pharmaceutically acceptable excipient. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one superagonistic CD28 antigen binding molecule and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intralesional, intravenous, intraarterial intramuscular, intrathecal or intraperitoneal injection. For injection, the TNF family ligand trimer-containing antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the superagonistic CD28 antigen binding molecule may be in powder form for constitution with a suitable vehicle, e.g., sterile pyrogen-free water, before use. Sterile injectable solutions are prepared by incorporating the fusion proteins of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

In addition to the compositions described previously, the superagonistic CD28 antigen binding molecule may also be formulated as a depot preparation. Such long acting formulations may be administered by implantation (for example subcutaneously or intramuscularly) or by intramuscular injection. Thus, for example, the superagonistic CD28 antigen binding molecule may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the superagonistic CD28 antigen binding molecule of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The superagonistic CD28 antigen binding molecule of the invention may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein may also contain more than one active ingredients as necessary for the particular indication being treated, preferably those with complementary activities that do not adversely affect each other. Such active ingredients are suitably present in combination in amounts that are effective for the purpose intended.

The formulations to be used for in vivo administration are generally sterile. Sterility may be readily accomplished, e.g., by filtration through sterile filtration membranes.

Therapeutic Methods and Compositions

Any of the superagonistic CD28 antigen binding molecules provided herein may be used in therapeutic methods, either alone or in combination.

In one aspect, a superagonistic CD28 antigen binding molecule for use as a medicament is provided. In further aspects, a superagonistic CD28 antigen binding molecule for use in treating cancer is provided. In certain aspects, a superagonistic CD28 antigen binding molecule for use in a method of treatment is provided. In certain aspects, herein is provided a superagonistic CD28 antigen binding molecule for use in a method of treating an individual having cancer comprising administering to the individual an effective amount of the superagonistic CD28 antigen binding molecule. In one such embodiment, the method further comprises administering to the individual an effective amount of at least one additional therapeutic agent.

In further aspects, a superagonistic CD28 antigen binding molecule as described herein for use in cancer immunotherapy is provided. In certain embodiments, a superagonistic CD28 antigen binding molecule for use in a method of cancer immunotherapy is provided. An “individual” according to any of the above aspects is preferably a human.

In a further aspect, herein is provided for the use of a superagonistic CD28 antigen binding molecule as described herein in the manufacture or preparation of a medicament. In one embodiment, the medicament is for treatment of cancer. In a further aspect, the medicament is for use in a method of treating cancer comprising administering to an individual having cancer an effective amount of the medicament. In one such aspect, the method further comprises administering to the individual an effective amount of at least one additional therapeutic agent, e.g., as described below. An “individual” according to any of the above aspects may be a human.

In a further aspect, herein is provided a method for treating a cancer. In one aspect, the method comprises administering to an individual having cancer an effective amount of a superagonistic CD28 antigen binding molecule. In one such aspect, the method further comprises administering to the individual an effective amount of at least one additional therapeutic agent, as described below. An “individual” according to any of the above aspects may be a human.

In a further aspect, herein are provided pharmaceutical formulations comprising any of the superagonistic CD28 antigen binding molecules as reported herein, e.g., for use in any of the above therapeutic methods. In one aspect, a pharmaceutical formulation comprises any of the superagonistic CD28 antigen binding molecules as reported herein and a pharmaceutically acceptable carrier. In another aspect, a pharmaceutical formulation comprises any of the superagonistic CD28 antigen binding molecules as reported herein and at least one additional therapeutic agent.

Antibodies as reported herein can be used either alone or in combination with other agents in a therapy. For instance, an antibody as reported herein may be co-administered with at least one additional therapeutic agent.

Such combination therapies noted above encompass combined administration (where two or more therapeutic agents are included in the same or separate formulations), and separate administration, in which case, administration of the antibody as reported herein can occur prior to, simultaneously, and/or following, administration of the additional therapeutic agent or agents. In one aspect, administration of the superagonistic CD28 antigen binding molecule and administration of an additional therapeutic agent occur within about one month, or within about one, two or three weeks, or within about one, two, three, four, five, or six days, of each other.

An antigen binding molecule as reported herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Superagonistic CD28 antigen binding molecules as described herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The superagonistic CD28 antigen binding molecule need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a superagonistic CD28 antigen binding molecule as described herein (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The superagonistic CD28 antigen binding molecule is suitably administered to the patient at one time or over a series of treatments. Depending on the type and severity of the disease, about 1 μg/kg to 15 mg/kg (e.g. 0.5 mg/kg-10 mg/kg) of superagonistic CD28 antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

Other Agents and Treatments

The superagonistic CD28 antigen binding molecules of the invention may be administered in combination with one or more other agents in therapy. For instance, an antigen binding molecule of the invention may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is another anti-cancer agent.

Such other agents are suitably present in combination in amounts that are effective for the purpose intended. The effective amount of such other agents depends on the amount of antigen binding molecule used, the type of disorder or treatment, and other factors discussed above. The superagonistic CD28 antigen binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the superagonistic CD28 antigen binding molecule of the invention can occur prior to, simultaneously, and/or following, administration of the additional therapeutic agent and/or adjuvant.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a superagonistic CD28 antigen binding molecule of the invention.

The label or package insert indicates that the composition is used for treating the condition of choice. Moreover, the article of manufacture may comprise (a) a first container with a composition contained therein, wherein the composition comprises a superagonistic CD28 antigen binding molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE 13
(Sequences)

| SEQ | | |
|---|---|---|
| ID | | |
| NO: | NAME | Sequence |
| 1 | hu CD28 | UniProt no. P10747, version 1 |
| | | MLRLLLALNL FPSIQVTGNK ILVKQSPMLV |
| | | AYDNAVNLSC KYSYNLFSRE FRASLHKGLD |
| | | SAVEVCVVYG NYSQQLQVYS KTGFNCDGKL |
| | | GNESVTFYLQ NLYVNQTDIY FCKIEVMYPP |
| | | PYLDNEKSNG TIIHVKGKHL CPSPLFPGPS |
| | | KPFWVLVVVG GVLACYSLLV TVAFIIFWVR |
| | | SKRSRLLHSD YMNMTPRRPG PTRKHYQPYA |
| | | PPRDFAAYRS |
| 2 | hu FAP | UniProt no. Q12884, version 168 |
| | | MKTWVKIVFG VATSAVLALL VMCIVLRPSR |
| | | VHNSEENTMR ALTLKDILNG TFSYKTFFPN |
| | | WISGQEYLHQ SADNNIVLYN IETGQSYTIL |
| | | SNRTMKSVNA SNYGLSPDRQ FVYLESDYSK |
| | | LWRYSYTATY YIYDLSNGEF VRGNELPRPI |
| | | QYLCWSPVGS KLAYVYQNNI YLKQRPGDPP |
| | | FQITFNGREN KIFNGIPDWV YEEEMLATKY |
| | | ALWWSPNGKF LAYAEFNDTD IPVIAYSYYG |
| | | DEQYPRTINI PYPKAGAKNP VVRIFIIDTT |
| | | YPAYVGPQEV PVPAMIASSD YYFSWLTWVT |
| | | DERVCLQWLK RVQNVSVLSI CDFREDWQTW |
| | | DCPKTQEHIE ESRTGWAGGF FVSTPVFSYD |
| | | AISYYKIFSD KDGYKHIHYI KDTVENAIQI |
| | | TSGKWEAINI FRVTQDSLFY SSNEFEEYPG |
| | | RRNIYRISIG SYPPSKKCVT CHLRKERCQY |
| | | YTASFSDYAK YYALVCYGPG IPISTLHDGR |
| | | TDQEIKILEE NKELENALKN IQLPKEEIKK |
| | | LEVDEITLWY KMILPPQFDR SKKYPLLIQV |
| | | YGGPCSQSVR SVFAVNWISY LASKEGMVIA |
| | | LVDGRGTAFQ GDKLLYAVYR KLGVYEVEDQ |
| | | ITAVRKFIEM GFIDEKRIAI WGWSYGGYVS |
| | | SLALASGTGL FKCGIAVAPV SSWEYYASVY |
| | | TERFMGLPTK DDNLEHYKNS TVMARAEYFR |
| | | NVDYLLIHGT ADDNVHFQNS AQIAKALVNA |
| | | QVDFQAMWYS DQNHGLSGLS TNHLYTHMTH |
| | | FLKQCFSLSD |
| 3 | hu CEA | UniProt accession no. P06731 |
| | | MESPSAPPHR WCIPWQRLLL TASLLTFWNP |
| | | PTTAKLTIES TPFNVAEGKE VLLLVHNLPQ |
| | | HLFGYSWYKG ERVDGNRQII GYVIGTQQAT |
| | | PGPAYSGREI IYPNASLLIQ NIIQNDTGFY |
| | | TLHVIKSDLV NEEATGQFRV YPELPKPSIS |
| | | SNNSKPVEDK DAVAFTCEPE TQDATYLWWV |
| | | NNQSLPVSPR LQLSNGNRTL TLFNVTRNDT |
| | | ASYKCETQNP VSARRSDSVI LNVLYGPDAP |
| | | TISPLNTSYR SGENLNLSCH AASNPPAQYS |
| | | WFVNGTFQQS TQELFIPNIT VNNSGSYTCQ |
| | | AHNSDTGLNR TTVTTITVYA EPPKPFITSN |
| | | NSNPVEDEDA VALTCEPEIQ NTTYLWWVNN |
| | | QSLPVSPRLQ LSNDNRTLTL LSVTRNDVGP |
| | | YECGIQNKLS VDHSDPVILN VLYGPDDPTI |
| | | SPSYTYYRPG VNLSLSCHAA SNPPAQYSWL |
| | | IDGNIQQHTQ ELFISNITEK NSGLYTCQAN |
| | | NSASGHSRTT VKTITVSAEL PKPSISSNNS |
| | | KPVEDKDAVA FTCEPEAQNT TYLWWVNGQS |
| | | LPVSPRLQLS NGNRTLTLFN VTRNDARAYV |
| | | CGIQNSVSAN RSDPVTLDVL YGPDTPIISP |
| | | PDSSYLSGAN LNLSCHSASN PSPQYSWRIN |
| | | GIPQQHTQVL FIAKITPNNN GTYACFVSNL |
| | | ATGRNNSIVK SITVSASGTS PGLSAGATVG |
| | | IMIGVLVGVA LI |
| 4 | FAP (28H1) CDR-H1 | SHAMS |
| 5 | FAP (28H1) CDR-H2 | AIWASGEQYYADSVKG |
| 6 | FAP (28H1) CDR-H3 | GWLGNFDY |
| 7 | FAP (28H1) CDR-L1 | RASQSVSRSYLA |
| 8 | FAP (28H1) CDR-L2 | GASTRAT |
| 9 | FAP (28H1) CDR-L3 | QQGQVIPPT |
| 10 | FAP (28H1) VH | EVQLLESGGGLVQPGGSLRLSCAASGFTFSSHAMSWVRQ |
| | | APGKGLEWVSAIWASGEQYYADSVKGRFTISRDNSKNTL |
| | | YLQMNSLRAEDTAVYYCAKGWLGNFDYWGQGTLVTVSS |
| 11 | FAP(28H1) VL | EIVLTQSPGTLSLSPGERATLSCRASQSVSRSYLAWYQQ |
| | | KPGQAPRLLIIGASTRATGIPDRFSGSGSGTDFTLTISR |
| | | LEPEDFAVYYCQQGQVIPPTFGQGTKVEIK |
| 12 | FAP(4B9) CDR-H1 | SYAMS |
| 13 | FAP(4B9) CDR-H2 | AIIGSGASTYYADSVKG |
| 14 | FAP(4B9) CDR-H3 | GWFGGFNY |
| 15 | FAP(4B9) CDR-L1 | RASQSVTSSYLA |
| 16 | FAP(4B9) CDR-L2 | VGSRRAT |
| 17 | FAP(4B9) CDR-L3 | QQGIMLPPT |
| 18 | FAP(4B9) VH | EVQLLESGGGLVQPGGSLRLSCAASGFTFSSYAMSWVRQ |
| | | APGKGLEWVSAIIGSGASTYYADSVKGRFTISRDNSKNT |
| | | LYLQMNSLRAEDTAVYYCAKGWFGGFNYWGQGTLVTVSS |
| 19 | FAP(4B9) VL | EIVLTQSPGTLSLSPGERATLSCRASQSVTSSYLAWYQQ |
| | | KPGQAPRLLINVGSRRATGIPDRFSGSGSGTDFTLTISR |
| | | LEPEDFAVYYCQQGIMLPPTFGQGTKVEIK |
| 20 | CD28(SA) CDR-H1 | SYYIH |
| 21 | CD28(SA) CDR-H2 | CIYPGNVNTNYNEKFKD |
| 22 | CD28(SA) CDR-H3 | SHYGLDWNFDV |
| 23 | CD28(SA) CDR-L1 | HASQNIYVWLN |
| 24 | CD28(SA) CDR-L2 | KASNLHT |
| 25 | CD28(SA) CDR-L3 | QQGQTYPYT |
| 26 | CD28(SA) VH | QVQLVQSGAEVKKPGASVKVSCKASGYTFTSYYIHWVRQ |
| | | APGQGLEWIGCIYPGNVNTNYNEKFKDRATLTVDTSIST |
| | | AYMELSRLRSDDTAVYFCTRSHYGLDWNFDVWGQGTTVT |
| | | VSS |
| 27 | CD28(SA) VL | DIQMTQSPSSLSASVGDRVTITCHASQNIYVWLNWYQQK |
| | | PGKAPKLLIYKASNLHTGVPSRFSGSGSGTDFTLTISSL |
| | | QPEDFATYYCQQGQTYPYTFGGGTKVEIK |
| 28 | CD28(mAb 9.3) CDR-H1 | DYGVH |
| 29 | CD28(mAb 9.3) CDR-H2 | VIWAGGGTNYNSALMS |
| 30 | CD28(mAb 9.3) CDR-H3 | DKGYSYYYSMDY |
| 31 | CD28(mAb 9.3) CDR-L1 | RASESVEYYVTSLMQ |
| 32 | CD28(mAb 9.3) CDR-L2 | AASNVES |
| 33 | CD28(mAb 9.3) CDR-L3 | QQSRKVPYT |
| 34 | CD28(mAb 9.3) VH | EVKLQQSGPGLVTPSQSLSITCTVSGFSLSDYGVHWVRQ |
| | | SPGQGLEWLGVIWAGGGTNYNSALMSRKSISKDNSKSQV |
| | | FLKMNSLQADDTAVYYCARDKGYSYYYSMDYWGQGTSVT |
| | | VSS |
| 35 | CD28(mAb 9.3) VL | DIELTQSPASLAVSLGQRATISCRASESVEYYVTSLMQW |
| | | YQQKPGQPPKLLIFAASNVESGVPARFSGSGSGTNFSLN |
| | | IHPVDEDDVAMYFCQQSRKVPYTFGGGTKLEIK |
| 36 | CD28 CDR-H1 consensus | SYYIH |
| 37 | CD28 CDR-H2 consensus | SIYPX₁X₂X₃X₄TNYNEKFKD, wherein |
| | | X₁ is G or R |
| | | X₂ is N or D |
| | | X₃ is V or G |
| | | X₄ is N or Q or A |
| 38 | CD28 CDR-H3 consensus | SHYGX₅DX₆NFDV, wherein |
| | | X₅ is L or A |
| | | X₆ is W or H or Y or F |
| 39 | CD28 CDR-L1 consensus | X₇ASQX₈IX₉X₁₀X₁₁LN, wherein |
| | | X₇ is H or R |
| | | X₈ is N or G |
| | | X₉ is Y or S |
| | | X₁₀ is V or N |
| | | X₁₁ is W or H or F or Y |
| 40 | CD28 CDR-L2 consensus | X₁₂X₁₃SX₁₄LX₁₅X₁₆, wherein |
| | | X₁₂ is K or Y |
| | | X₁₃ is A or T |
| | | X₁₄ is N or S |
| | | X₁₅ is H or Y |
| | | X₁₆ is T or S |
| 41 | CD28 CDR-L3 consensus | QQX₁₇QTYPYT, wherein |
| | | X₁₇ is G or A |
| 42 | CD28 VH variant a | QVQLVQSGAEVKKPGASVKVSCKASGYTFTSYYIHWVRQAPGQ |
| | | GLEWIGSIYPGNVNTNYNEKFKDRATLTVDTSISTAYMELSRL |
| | | RSDDTAVYFCTRSHYGLDWNFDVWGQGTTVTVSS |
| 43 | CD28 VH variant b | QVQLVQSGAEVKKPGASVKVSCKASGYTFTSYYIHWVRQAPGQ |
| | | GLEWIGSIYPGNVQTNYNEKFKDRATLTVDTSISTAYMELSRL |
| | | RSDDTAVYFCTRSHYGLDHNFDVWGQGTTVTVSS |
| 44 | CD28 VH variant c | QVQLVQSGAEVKKPGASVKVSCKASGYTFTSYYIHWVRQAPGQ |
| | | GLEWIGSIYPGNVQTNYNEKFKDRATLTVDTSISTAYMELSRL |
| | | RSDDTAVYFCTRSHYGADHNFDVWGQGTTVTVSS |
| 45 | CD28 VH variant d | QVQLVQSGAEVKKPGASVKVSCKASGYTFTSYYIHWVRQAPGQ |
| | | GLEWIGSIYPRDGQTNYNEKFKDRATLTVDTSISTAYMELSRL |
| | | RSDDTAVYFCTRSHYGLDYNFDVWGQGTTVTVSS |
| 46 | CD28 VH variant e | QVQLVQSGAEVKKPGASVKVSCKASGYTFTSYYIHWVRQAPGQ |
| | | GLEWIGSIYPGNVQTNYNEKFKDRATLTVDTSISTAYMELSRL |
| | | RSDDTAVYFCTRSHYGLDWNFDVWGQGTTVTVSS |
| 47 | CD28 VH variant f | QVQLVQSGAEVKKPGASVKVSCKASGYTFTSYYIHWVRQAPGQ |
| | | GLEWIGSIYPGNVQTNYNEKFKDRATLTVDTSISTAYMELSRL |
| | | RSDDTAVYFCTRSHYGLDFNFDVWGQGTTVTVSS |
| 48 | CD28 VH variant g | QVQLVQSGAEVKKPGASVKVSCKASGYTFTSYYIHWVRQAPGQ |
| | | GLEWIGSIYPRNVQTNYNEKFKDRATLTVDTSISTAYMELSRL |
| | | RSDDTAVYFCTRSHYGLDHNFDVWGQGTTVTVSS |
| 49 | CD28 VH variant h | QVQLVQSGAEVKKPGASVKVSCKASGYTFTSYYIHWVRQAPGQ |
| | | GLEWIGSIYPRDVQTNYNEKFKDRATLTVDTSISTAYMELSRL |
| | | RSDDTAVYFCTRSHYGLDHNFDVWGQGTTVTVSS |
| 50 | CD28 VH variant i | EVQLVESGGGLVQPGGSLRLSCAASGFTFTSYYIHWVRQAPGK |
| | | GLEWVASIYPGNVNTRYADSVKGRFTISADTSKNTAYLQMNSL |
| | | RAEDTAVYYCTRSHYGLDWNFDVWGQGTTVTVSS |
| 51 | CD28 VH variant j | EVQLVESGGGLVQPGGSLRLSCAASGFTFTSYYIHWVRQAPGK |
| | | GLEWVASIYPGNVATRYADSVKGRFTISADTSKNTAYLQMNSL |
| | | RAEDTAVYYCTRSHYGLDWNFDVWGQGTTVTVSS |
| 52 | CD28 VL variant k | DIQMTQSPSSLSASVGDRVTITCHASQNIYVHLNWYQQKPGKA |
| | | PKLLIYKASNLHTGVPSRFSGSGSGTDFTLTISSLQPEDFATY |
| | | YCQQAQTYPYTFGGGTKVEIK |
| 53 | CD28 VL variant l | DIQMTQSPSSLSASVGDRVTITCHASQNIYVFLNWYQQKPGKA |
| | | PKLLIYKASNLHTGVPSRFSGSGSGTDFTLTISSLQPEDFATY |
| | | YCQQGQTYPYTFGGGTKVEIK |
| 54 | CD28 VL variant m | DIQMTQSPSSLSASVGDRVTITCHASQNIYVYLNWYQQKPGKA |
| | | PKLLIYKASNLHTGVPSRFSGSGSGTDFTLTISSLQPEDFATY |
| | | YCQQGQTYPYTFGGGTKVEIK |
| 55 | CD28 VL variant n | DIQMTQSPSSLSASVGDRVTITCHASQGISNYLNWYQQKPGKA |
| | | PKLLIYKASNLHTGVPSRFSGSGSGTDFTLTISSLQPEDFATY |
| | | YCQQGQTYPYTFGGGTKVEIK |
| 56 | CD28 VL variant o | DIQMTQSPSSLSASVGDRVTITCHASQNIYVWLNWYQQKPGKA |
| | | PKLLIYYTSSLHSGVPSRFSGSGSGTDFTLTISSLQPEDFATY |
| | | YCQQGQTYPYTFGGGTKVEIK |
| 57 | CD28 VL variant p | DIQMTQSPSSLSASVGDRVTITCHASQGISNYLNWYQQKPGKA |
| | | PKLLIYYTSSLHSGVPSRFSGSGSGTDFTLTISSLQPEDFATY |
| | | YCQQGQTYPYTFGGGTKVEIK |
| 58 | CD28 VL variant q | DIQMTQSPSSLSASVGDRVTITCHASQGISNHLNWYQQKPGKA |
| | | PKLLIYKASNLHTGVPSRFSGSGSGTDFTLTISSLQPEDFATY |
| | | YCQQGQTYPYTFGGGTKVEIK |
| 59 | CD28 VL variant r | DIQMTQSPSSLSASVGDRVTITCHASQGIYVYLNWYQQKPGKA |
| | | PKLLIYKASNLHTGVPSRFSGSGSGTDFTLTISSLQPEDFATY |
| | | YCQQGQTYPYTFGGGTKVEIK |
| 60 | CD28 VL variant s | DIQMTQSPSSLSASVGDRVTITCHASQGISVYLNWYQQKPGKA |
| | | PKLLIYKASNLHTGVPSRFSGSGSGTDFTLTISSLQPEDFATY |
| | | YCQQGQTYPYTFGGGTKVEIK |
| 61 | CD28 VL variant t | DIQMTQSPSSLSASVGDRVTITCRASQNIYVWLNWYQQKPGKA |
| | | PKLLIYKASNLYSGVPSRFSGSRSGTDFTLTISSLQPEDFATY |
| | | YCQQGQTYPYTFGQGTKLEIK |
| 62 | CD28(SA) light chain | DIQMTQSPSSLSASVGDRVTITCHASQNIYVWLNWYQQK |
| | | PGKAPKLLIYKASNLHTGVPSRFSGSGSGTDFTLTISSL |
| | | QPEDFATYYCQQGQTYPYTFGGGTKVEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 63 | CD28(SA) hu IgG4 heavy | QVQLVQSGAEVKKPGASVKVSCKASGYTFTSYYIHWVRQ |
| | chain | APGQGLEWIGCIYPGNVNTNYNEKFKDRATLTVDTSIST |
| | | AYMELSRLRSDDTAVYFCTRSHYGLDWNFDVWGQGTTVT |
| | | VSSASTKGPSVFPLAPCSRSTSESTAALGCLVKDYFPEP |
| | | VTVSWNSGALTSGVHTFPAVLQSSGLYSLSSVVTVPSSS |
| | | LGTKTYTCNVDHKPSNTKVDKRVESKYGPPCPSCPAPEF |
| | | LGGPSVFLFPPKPKDTLMISRTPEVTCVVVDVSQEDPEV |
| | | QFNWYVDGVEVHNAKTKPREEQFNSTYRVVSVLTVLHQD |
| | | WLNGKEYKCKVSNKGLPSSIEKTISKAKGQPREPQVYTL |
| | | PPSQEEMTKNQVSLTCLVKGFYPSDIAVEWESNGQPENN |
| | | YKTTPPVLDSDGSFFLYSRLTVDKSRWQEGNVFSCSVMH |
| | | EALHNHYTQKSLSLSLGK |
| 64 | CD28(SA) hu IgG1 | QVQLVQSGAEVKKPGASVKVSCKASGYTFTSYYIHWVRQ |
| | PGLALA heavy chain | APGQGLEWIGCIYPGNVNTNYNEKFKDRATLTVDTSIST |
| | | AYMELSRLRSDDTAVYFCTRSHYGLDWNFDVWGQGTTVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPSRDELTKNQVSLTCLVKGFYPSDIAVEWESNGQP |
| | | ENNYKTTPPVLDSDGSFFLYSKLTVDKSRWQQGNVFSCS |
| | | VMHEALHNHYTQKSLSLSP |
| 65 | CD28(SA) hu IgG1 light | DIQMTQSPSSLSASVGDRVTITCHASQNIYVWLNWYQQK |
| | chain “RK” | PGKAPKLLIYKASNLHTGVPSRFSGSGSGTDFTLTISSL |
| | | QPEDFATYYCQQGQTYPYTFGGGTKVEIKRTVAAPSVFI |
| | | FPPSDRKLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 66 | CD28(SA) hu IgG1 | QVQLVQSGAEVKKPGASVKVSCKASGYTFTSYYIHWVRQ |
| | PGLALA Fc knob | APGQGLEWIGCIYPGNVNTNYNEKFKDRATLTVDTSIST |
| | | AYMELSRLRSDDTAVYFCTRSHYGLDWNFDVWGQGTTVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSP |
| 67 | FAP(4B9) VL-CH hu IgG1 | EIVLTQSPGTLSLSPGERATLSCRASQSVTSSYLAWYQQ |
| | PGLALA Fc hole | KPGQAPRLLINVGSRRATGIPDRFSGSGSGTDFTLTISR |
| | | LEPEDFAVYYCQQGIMLPPTFGQGTKVEIKSSASTKGPS |
| | | VFPLAPSSKSTSGGTAALGCLVKDYFPEPVTVSWNSGAL |
| | | TSGVHTFPAVLQSSGLYSLSSVVTVPSSSLGTQTYICNV |
| | | NHKPSNTKVDKKVEPKSCDKTHTCPPCPAPEAAGGPSVF |
| | | LFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALGAPIEKTISKAKGQPREPQVCTLPPSRDEL |
| | | TKNQVSLSCAVKGFYPSDIAVEWESNGQPENNYKTTPPV |
| | | LDSDGSFELVSKLTVDKSRWQQGNVESCSVMHEALHNHY |
| | | TQKSLSLSP |
| 68 | FAP(4B9) VH-Ckappa | EVQLLESGGGLVQPGGSLRLSCAASGETFSSYAMSWVRQ |
| | | APGKGLEWVSAIIGSGASTYYADSVKGRFTISRDNSKNT |
| | | LYLQMNSLRAEDTAVYYCAKGWEGGENYWGQGTLVTVSS |
| | | ASVAAPSVFIFPPSDEQLKSGTASVVCLLNNFYPREAKV |
| | | QWKVDNALQSGNSQESVTEQDSKDSTYSLSSTLTLSKAD |
| | | YEKHKVYACEVTHQGLSSPVTKSFNRGEC |
| 69 | CD28(SA) VHCH-VHCH | QVQLVQSGAEVKKPGASVKVSCKASGYTFTSYYIHWVRQ |
| | hu IgG1 Fc knob FAP(4B9) | APGQGLEWIGCIYPGNVNTNYNEKFKDRATLTVDTSIST |
| | VH PGLALA | AYMELSRLRSDDTAVYFCTRSHYGLDWNFDVWGQGTTVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDGGGGSGGGGS |
| | | QVQLVQSGAEVKKPGASVKVSCKASGYTFTSYYIHWVRQ |
| | | APGQGLEWIGCIYPGNVNTNYNEKFKDRATLTVDTSIST |
| | | AYMELSRLRSDDTAVYFCTRSHYGLDWNFDVWGQGTTVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSPGGGGGSGGGGSGGGGSGGGG |
| | | SEVQLLESGGGLVQPGGSLRLSCAASGETFSSYAMSWVR |
| | | QAPGKGLEWVSAIIGSGASTYYADSVKGRFTISRDNSKN |
| | | TLYLQMNSLRAEDTAVYYCAKGWEGGENYWGQGTLVTVS |
| | | S |
| 70 | CD28(SA) VHCH-VHCH | QVQLVQSGAEVKKPGASVKVSCKASGYTFTSYYIHWVRQ |
| | hu IgG1 Fc hole FAP(4B9) | APGQGLEWIGCIYPGNVNTNYNEKFKDRATLTVDTSIST |
| | VL PGLALA | AYMELSRLRSDDTAVYFCTRSHYGLDWNFDVWGQGTTVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDGGGGSGGGGS |
| | | QVQLVQSGAEVKKPGASVKVSCKASGYTFTSYYIHWVRQ |
| | | APGQGLEWIGCIYPGNVNTNYNEKFKDRATLTVDTSIST |
| | | AYMELSRLRSDDTAVYFCTRSHYGLDWNFDVWGQGTTVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | CTLPPSRDELTKNQVSLSCAVKGFYPSDIAVEWESNGQP |
| | | ENNYKTTPPVLDSDGSFFLVSKLTVDKSRWQQGNVFSCS |
| | | VMHEALHNHYTQKSLSLSPGGGGGSGGGGSGGGGSGGGG |
| | | SEIVLTQSPGTLSLSPGERATLSCRASQSVTSSYLAWYQ |
| | | QKPGQAPRLLINVGSRRATGIPDRFSGSGSGTDFTLTIS |
| | | RLEPEDFAVYYCQQGIMLPPTFGQGTKVEIK |
| 71 | CD28(SA) VHCH-hu IgG1 | QVQLVQSGAEVKKPGASVKVSCKASGYTFTSYYIHWVRQ |
| | Fc knob FAP(4B9) VH | APGQGLEWIGCIYPGNVNTNYNEKFKDRATLTVDTSIST |
| | | AYMELSRLRSDDTAVYFCTRSHYGLDWNFDVWGQGTTVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSPGGGGGSGGGGSGGGGSGGGG |
| | | SEVQLLESGGGLVQPGGSLRLSCAASGETFSSYAMSWVR |
| | | QAPGKGLEWVSAIIGSGASTYYADSVKGRFTISRDNSKN |
| | | TLYLQMNSLRAEDTAVYYCAKGWEGGFNYWGQGTLVTVS |
| | | S |
| 72 | CD28(SA) VHCH-hu IgG1 | QVQLVQSGAEVKKPGASVKVSCKASGYTFTSYYIHWVRQ |
| | Fc hole FAP(4B9) VL | APGQGLEWIGCIYPGNVNTNYNEKFKDRATLTVDTSIST |
| | | AYMELSRLRSDDTAVYFCTRSHYGLDWNFDVWGQGTTVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | CTLPPSRDELTKNQVSLSCAVKGFYPSDIAVEWESNGQP |
| | | ENNYKTTPPVLDSDGSFFLVSKLTVDKSRWQQGNVFSCS |
| | | VMHEALHNHYTQKSLSLSPGGGGGSGGGGSGGGGSGGGG |
| | | SEIVLTQSPGTLSLSPGERATLSCRASQSVTSSYLAWYQ |
| | | QKPGQAPRLLINVGSRRATGIPDRFSGSGSGTDFTLTIS |
| | | RLEPEDFAVYYCQQGIMLPPTFGQGTKVEIK |
| 73 | CD28(SA) VHCH “EE”-hu | QVQLVQSGAEVKKPGASVKVSCKASGYTFTSYYIHWVRQ |
| | IgG1 Fc PGLALA | APGQGLEWIGCIYPGNVNTNYNEKFKDRATLTVDTSIST |
| | FAP(4B9) VHCL | AYMELSRLRSDDTAVYFCTRSHYGLDWNFDVWGQGTTVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPSRDELTKNQVSLTCLVKGFYPSDIAVEWESNGQP |
| | | ENNYKTTPPVLDSDGSFFLYSKLTVDKSRWQQGNVFSCS |
| | | VMHEALHNHYTQKSLSLSPGGGGGSGGGGSGGGGSGGGG |
| | | SEVQLLESGGGLVQPGGSLRLSCAASGETFSSYAMSWVR |
| | | QAPGKGLEWVSAIIGSGASTYYADSVKGRFTISRDNSKN |
| | | TLYLQMNSLRAEDTAVYYCAKGWEGGENYWGQGTLVTVS |
| | | SASVAAPSVFIFPPSDEQLKSGTASVVCLLNNFYPREAK |
| | | VQWKVDNALQSGNSQESVTEQDSKDSTYSLSSTLTLSKA |
| | | DYEKHKVYACEVTHQGLSSPVTKSFNRGEC |
| 74 | FAP(4B9) VLCH1 | EIVLTQSPGTLSLSPGERATLSCRASQSVTSSYLAWYQQ |
| | | KPGQAPRLLINVGSRRATGIPDRFSGSGSGTDFTLTISR |
| | | LEPEDFAVYYCQQGIMLPPTFGQGTKVEIKSSASTKGPS |
| | | VFPLAPSSKSTSGGTAALGCLVKDYFPEPVTVSWNSGAL |
| | | TSGVHTFPAVLQSSGLYSLSSVVTVPSSSLGTQTYICNV |
| | | NHKPSNTKVDKKVEPKSCD |
| 75 | CD28(SA) VLCH1- | DIQMTQSPSSLSASVGDRVTITCHASQNIYVWLNWYQQK |
| | FAP(4B9) VHCH1 “EE”- | PGKAPKLLIYKASNLHTGVPSRFSGSGSGTDFTLTISSL |
| | hu IgG1 Fc knob PGLALA | QPEDFATYYCQQGQTYPYTFGGGTKVEIKSSASTKGPSV |
| | | FPLAPSSKSTSGGTAALGCLVKDYFPEPVTVSWNSGALT |
| | | SGVHTFPAVLQSSGLYSLSSVVTVPSSSLGTQTYICNVN |
| | | HKPSNTKVDKKVEPKSCDGGGGSGGGGSEVQLLESGGGL |
| | | VQPGGSLRLSCAASGETFSSYAMSWVRQAPGKGLEWVSA |
| | | IIGSGASTYYADSVKGRFTISRDNSKNTLYLQMNSLRAE |
| | | DTAVYYCAKGWFGGFNYWGQGTLVTVSSASTKGPSVFPL |
| | | APSSKSTSGGTAALGCLVEDYFPEPVTVSWNSGALTSGV |
| | | HTFPAVLQSSGLYSLSSVVTVPSSSLGTQTYICNVNHKP |
| | | SNTKVDEKVEPKSCDKTHTCPPCPAPEAAGGPSVFLFPP |
| | | KPKDTLMISRTPEVTCVVVDVSHEDPEVKFNWYVDGVEV |
| | | HNAKTKPREEQYNSTYRVVSVLTVLHQDWLNGKEYKCKV |
| | | SNKALGAPIEKTISKAKGQPREPQVYTLPPCRDELTKNQ |
| | | VSLWCLVKGFYPSDIAVEWESNGQPENNYKTTPPVLDSD |
| | | GSFFLYSKLTVDKSRWQQGNVFSCSVMHEALHNHYTQKS |
| | | LSLSP |
| 76 | FAP(4B9) VHCH1 “EE”- | EVQLLESGGGLVQPGGSLRLSCAASGFTFSSYAMSWVRQ |
| | hu IgG1 Fc hole PGLALA | APGKGLEWVSAIIGSGASTYYADSVKGRFTISRDNSKNT |
| | | LYLQMNSLRAEDTAVYYCAKGWEGGENYWGQGTLVTVSS |
| | | ASTKGPSVFPLAPSSKSTSGGTAALGCLVEDYFPEPVTV |
| | | SWNSGALTSGVHTFPAVLQSSGLYSLSSVVTVPSSSLGT |
| | | QTYICNVNHKPSNTKVDEKVEPKSCDKTHTCPPCPAPEA |
| | | AGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALGAPIEKTISKAKGQPREPQVCTL |
| | | PPSRDELTKNQVSLSCAVKGFYPSDIAVEWESNGQPENN |
| | | YKTTPPVLDSDGSFFLVSKLTVDKSRWQQGNVFSCSVMH |
| | | EALHNHYTQKSLSLSP |
| 77 | CD28(SA) VHCL | QVQLVQSGAEVKKPGASVKVSCKASGYTFTSYYIHWVRQ |
| | | APGQGLEWIGCIYPGNVNTNYNEKFKDRATLTVDTSIST |
| | | AYMELSRLRSDDTAVYFCTRSHYGLDWNFDVWGQGTTVT |
| | | VSSASVAAPSVFIFPPSDEQLKSGTASVVCLLNNFYPRE |
| | | AKVQWKVDNALQSGNSQESVTEQDSKDSTYSLSSTLTLS |
| | | KADYEKHKVYACEVTHQGLSSPVTKSFNRGEC |
| 78 | FAP(4B9) VLCL “RK” | EIVLTQSPGTLSLSPGERATLSCRASQSVTSSYLAWYQQ |
| | | KPGQAPRLLINVGSRRATGIPDRFSGSGSGTDFTLTISR |
| | | LEPEDFAVYYCQQGIMLPPTFGQGTKVEIKRTVAAPSVF |
| | | IFPPSDRKLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 79 | hu IgG1 Fc hole PGLALA | DKTHTCPPCPAPEAAGGPSVFLFPPKPKDTLMISRTPEV |
| | | TCVVVDVSHEDPEVKFNWYVDGVEVHNAKTKPREEQYNS |
| | | TYRVVSVLTVLHQDWLNGKEYKCKVSNKALGAPIEKTIS |
| | | KAKGQPREPQVCTLPPSRDELTKNQVSLSCAVKGFYPSD |
| | | IAVEWESNGQPENNYKTTPPVLDSDGSFFLVSKLTVDKS |
| | | RWQQGNVFSCSVMHEALHNHYTQKSLSLSP |
| 80 | hu IgG1 Fc knob- | DKTHTCPPCPAPEAAGGPSVFLFPPKPKDTLMISRTPEV |
| | FAP(4B9) VH | TCVVVDVSHEDPEVKFNWYVDGVEVHNAKTKPREEQYNS |
| | | TYRVVSVLTVLHQDWLNGKEYKCKVSNKALGAPIEKTIS |
| | | KAKGQPREPQVYTLPPCRDELTKNQVSLWCLVKGFYPSD |
| | | IAVEWESNGQPENNYKTTPPVLDSDGSFFLYSKLTVDKS |
| | | RWQQGNVFSCSVMHEALHNHYTQKSLSLSPGGGGGSGGG |
| | | GSGGGGSGGGGSEVQLLESGGGLVQPGGSLRLSCAASGF |
| | | TESSYAMSWVRQAPGKGLEWVSAIIGSGASTYYADSVKG |
| | | RFTISRDNSKNTLYLQMNSLRAEDTAVYYCAKGWFGGFN |
| | | YWGQGTLVTVSS |
| 81 | CD28(SA) VHCH1 “EE”- | QVQLVQSGAEVKKPGASVKVSCKASGYTFTSYYIHWVRQ |
| | hu IgG1 Fc PGLALA | APGQGLEWIGCIYPGNVNTNYNEKFKDRATLTVDTSIST |
| | CEA(Medi-565) VHCL | AYMELSRLRSDDTAVYFCTRSHYGLDWNFDVWGQGTTVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPSRDELTKNQVSLTCLVKGFYPSDIAVEWESNGQP |
| | | ENNYKTTPPVLDSDGSFFLYSKLTVDKSRWQQGNVFSCS |
| | | VMHEALHNHYTQKSLSLSPGGGGGSGGGGSGGGGSGGGG |
| | | SEVQLVESGGGLVQPGRSLRLSCAASGFTVSSYWMHWVR |
| | | QAPGKGLEWVGFIRNKANGGTTEYAASVKGRFTISRDDS |
| | | KNTLYLQMNSLRAEDTAVYYCARDRGLRFYFDYWGQGTT |
| | | VTVSSASVAAPSVFIFPPSDEQLKSGTASVVCLLNNFYP |
| | | REAKVQWKVDNALQSGNSQESVTEQDSKDSTYSLSSTLT |
| | | LSKADYEKHKVYACEVTHQGLSSPVTKSFNRGEC |
| 82 | CEA VLCH1 | QAVLTQPASLSASPGASASLTCTLRRGINVGAYSIYWYQ |
| | | QKPGSPPQYLLRYKSDSDKQQGSGVSSRFSASKDASANA |
| | | GILLISGLQSEDEADYYCMIWHSGASAVFGGGTKLTVLS |
| | | SASTKGPSVFPLAPSSKSTSGGTAALGCLVKDYFPEPVT |
| | | VSWNSGALTSGVHTFPAVLQSSGLYSLSSVVTVPSSSLG |
| | | TQTYICNVNHKPSNTKVDKKVEPKSC |
| 83 | CD28(SA) VHCH1-hu | QVQLVQSGAEVKKPGASVKVSCKASGYTFTSYYIHWVRQ |
| | IgG1 Fc knob CEA VH | APGQGLEWIGCIYPGNVNTNYNEKFKDRATLTVDTSIST |
| | | AYMELSRLRSDDTAVYFCTRSHYGLDWNFDVWGQGTTVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSPGGGGGSGGGGSGGGGSGGGG |
| | | SEVQLVESGGGLVQPGRSLRLSCAASGFTVSSYWMHWVR |
| | | QAPGKGLEWVGFIRNKANGGTTEYAASVKGRFTISRDDS |
| | | KNTLYLQMNSLRAEDTAVYYCARDRGLRFYFDYWGQGTT |
| | | VTVS |
| 84 | CD28(SA) VHCH1-hu | QVQLVQSGAEVKKPGASVKVSCKASGYTFTSYYIHWVRQ |
| | IgG1 Fc hole CEA VL | APGQGLEWIGCIYPGNVNTNYNEKFKDRATLTVDTSIST |
| | | AYMELSRLRSDDTAVYFCTRSHYGLDWNFDVWGQGTTVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | CTLPPSRDELTKNQVSLSCAVKGFYPSDIAVEWESNGQP |
| | | ENNYKTTPPVLDSDGSFFLVSKLTVDKSRWQQGNVFSCS |
| | | VMHEALHNHYTQKSLSLSPGGGGGSGGGGSGGGGSGGGG |
| | | SQAVLTQPASLSASPGASASLTCTLRRGINVGAYSIYWY |
| | | QQKPGSPPQYLLRYKSDSDKQQGSGVSSRFSASKDASAN |
| | | AGILLISGLQSEDEADYYCMIWHSGASAVFGGGTKLTVL |
| 85 | CD28(SA) VHCH1 “EE”- | QVQLVQSGAEVKKPGASVKVSCKASGYTFTSYYIHWVRQ |
| | hu IgG1 Fc hole PGLALA | APGQGLEWIGCIYPGNVNTNYNEKFKDRATLTVDTSIST |
| | HYRF | AYMELSRLRSDDTAVYFCTRSHYGLDWNFDVWGQGTTVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | CTLPPSRDELTKNQVSLSCAVKGFYPSDIAVEWESNGQP |
| | | ENNYKTTPPVLDSDGSFFLVSKLTVDKSRWQQGNVFSCS |
| | | VMHEALHNRFTQKSLSLSP |
| 86 | hu IgG1 Fc knob PGLALA | DKTHTCPPCPAPEAAGGPSVFLFPPKPKDTLMISRTPEV |
| | | TCVVVDVSHEDPEVKFNWYVDGVEVHNAKTKPREEQYNS |
| | | TYRVVSVLTVLHQDWLNGKEYKCKVSNKALGAPIEKTIS |
| | | KAKGQPREPQVYTLPPCRDELTKNQVSLWCLVKGFYPSD |
| | | IAVEWESNGQPENNYKTTPPVLDSDGSFFLYSKLTVDKS |
| | | RWQQGNVFSCSVMHEALHNHYTQKSLSLSP |
| 87 | CEA VL-CH hu IgG1 | QAVLTQPASLSASPGASASLTCTLRRGINVGAYSIYWYQ |
| | PGLALA Fc hole | QKPGSPPQYLLRYKSDSDKQQGSGVSSRFSASKDASANA |
| | | GILLISGLQSEDEADYYCMIWHSGASAVFGGGTKLTVLS |
| | | SASTKGPSVFPLAPSSKSTSGGTAALGCLVKDYFPEPVT |
| | | VSWNSGALTSGVHTFPAVLQSSGLYSLSSVVTVPSSSLG |
| | | TQTYICNVNHKPSNTKVDKKVEPKSCDKTHTCPPCPAPE |
| | | AAGGPSVFLFPPKPKDTLMISRTPEVTCVVVDVSHEDPE |
| | | VKFNWYVDGVEVHNAKTKPREEQYNSTYRVVSVLTVLHQ |
| | | DWLNGKEYKCKVSNKALGAPIEKTISKAKGQPREPQVCT |
| | | LPPSRDELTKNQVSLSCAVKGFYPSDIAVEWESNGQPEN |
| | | NYKTTPPVLDSDGSFFLVSKLTVDKSRWQQGNVFSCSVM |
| | | HEALHNHYTQKSLSLSP |
| 88 | CEA VH-CL | EVQLVESGGGLVQPGRSLRLSCAASGFTVSSYWMHWVRQ |
| | | APGKGLEWVGFIRNKANGGTTEYAASVKGRFTISRDDSK |
| | | NTLYLQMNSLRAEDTAVYYCARDRGLRFYFDYWGQGTTV |
| | | TVSSASVAAPSVFIFPPSDEQLKSGTASVVCLLNNFYPR |
| | | EAKVQWKVDNALQSGNSQESVTEQDSKDSTYSLSSTLTL |
| | | SKADYEKHKVYACEVTHQGLSSPVTKSFNRGEC |
| 89 | CD28(mAb 9.3) light chain | DIELTQSPASLAVSLGQRATISCRASESVEYYVTSLMQW |
| | | YQQKPGQPPKLLIFAASNVESGVPARFSGSGSGTNFSLN |
| | | IHPVDEDDVAMYFCQQSRKVPYTFGGGTKLEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 90 | CD28(mAb 9.3) hu IgG1 | EVKLQQSGPGLVTPSQSLSITCTVSGFSLSDYGVHWVRQ |
| | PGLALA heavy chain | SPGQGLEWLGVIWAGGGTNYNSALMSRKSISKDNSKSQV |
| | | FLKMNSLQADDTAVYYCARDKGYSYYYSMDYWGQGTSVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPSRDELTKNQVSLTCLVKGFYPSDIAVEWESNGQP |
| | | ENNYKTTPPVLDSDGSFFLYSKLTVDKSRWQQGNVFSCS |
| | | VMHEALHNHYTQKSLSLSP |
| 91 | CD28(mAb 9.3) hu IgG1 | EVKLQQSGPGLVTPSQSLSITCTVSGFSLSDYGVHWVRQ |
| | PGLALA Fc knob “EE” | SPGQGLEWLGVIWAGGGTNYNSALMSRKSISKDNSKSQV |
| | | FLKMNSLQADDTAVYYCARDKGYSYYYSMDYWGQGTSVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSP |
| 92 | CD28(mAb 9.3) hu IgG1 | DIELTQSPASLAVSLGQRATISCRASESVEYYVTSLMQW |
| | light chain “RK” | YQQKPGQPPKLLIFAASNVESGVPARFSGSGSGTNFSLN |
| | | IHPVDEDDVAMYFCQQSRKVPYTFGGGTKLEIKRTVAAP |
| | | SVFIFPPSDRKLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 93 | CD28(mAb 9.3) VHCH- | EVKLQQSGPGLVTPSQSLSITCTVSGFSLSDYGVHWVRQ |
| | VHCH hu IgG1 Fc knob | SPGQGLEWLGVIWAGGGTNYNSALMSRKSISKDNSKSQV |
| | FAP(4B9) VH PGLALA | FLKMNSLQADDTAVYYCARDKGYSYYYSMDYWGQGTSVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDGGGGSGGGGS |
| | | EVKLQQSGPGLVTPSQSLSITCTVSGFSLSDYGVHWVRQ |
| | | SPGQGLEWLGVIWAGGGTNYNSALMSRKSISKDNSKSQV |
| | | FLKMNSLQADDTAVYYCARDKGYSYYYSMDYWGQGTSVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSPGGGGGSGGGGSGGGGSGGGG |
| | | SEVQLLESGGGLVQPGGSLRLSCAASGFTFSSYAMSWVR |
| | | QAPGKGLEWVSAIIGSGASTYYADSVKGRFTISRDNSKN |
| | | TLYLQMNSLRAEDTAVYYCAKGWFGGFNYWGQGTLVTVS |
| | | S |
| 94 | CD28(mAb 9.3) VHCH- | EVKLQQSGPGLVTPSQSLSITCTVSGFSLSDYGVHWVRQ |
| | VHCH hu IgG1 Fc hole | SPGQGLEWLGVIWAGGGTNYNSALMSRKSISKDNSKSQV |
| | FAP(4B9) VL PGLALA | FLKMNSLQADDTAVYYCARDKGYSYYYSMDYWGQGTSVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDGGGGSGGGGS |
| | | EVKLQQSGPGLVTPSQSLSITCTVSGFSLSDYGVHWVRQ |
| | | SPGQGLEWLGVIWAGGGTNYNSALMSRKSISKDNSKSQV |
| | | FLKMNSLQADDTAVYYCARDKGYSYYYSMDYWGQGTSVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | CTLPPSRDELTKNQVSLSCAVKGFYPSDIAVEWESNGQP |
| | | ENNYKTTPPVLDSDGSFFLVSKLTVDKSRWQQGNVFSCS |
| | | VMHEALHNHYTQKSLSLSPGGGGGSGGGGSGGGGSGGGG |
| | | SEIVLTQSPGTLSLSPGERATLSCRASQSVTSSYLAWYQ |
| | | QKPGQAPRLLINVGSRRATGIPDRFSGSGSGTDFTLTIS |
| | | RLEPEDFAVYYCQQGIMLPPTFGQGTKVEIK |
| 95 | CD28(mAb 9.3) VHCH-hu | EVKLQQSGPGLVTPSQSLSITCTVSGFSLSDYGVHWVRQ |
| | IgG1 Fc knob FAP(4B9) | SPGQGLEWLGVIWAGGGTNYNSALMSRKSISKDNSKSQV |
| | VH | FLKMNSLQADDTAVYYCARDKGYSYYYSMDYWGQGTSVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSPGGGGGSGGGGSGGGGSGGGG |
| | | SEVQLLESGGGLVQPGGSLRLSCAASGFTFSSYAMSWVR |
| | | QAPGKGLEWVSAIIGSGASTYYADSVKGRFTISRDNSKN |
| | | TLYLQMNSLRAEDTAVYYCAKGWFGGFNYWGQGTLVTVS |
| | | S |
| 96 | CD28(mAb 9.3) VHCH-hu | EVKLQQSGPGLVTPSQSLSITCTVSGFSLSDYGVHWVRQ |
| | IgG1 Fc hole FAP(4B9) VL | SPGQGLEWLGVIWAGGGTNYNSALMSRKSISKDNSKSQV |
| | | FLKMNSLQADDTAVYYCARDKGYSYYYSMDYWGQGTSVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | CTLPPSRDELTKNQVSLSCAVKGFYPSDIAVEWESNGQP |
| | | ENNYKTTPPVLDSDGSFFLVSKLTVDKSRWQQGNVFSCS |
| | | VMHEALHNHYTQKSLSLSPGGGGGSGGGGSGGGGSGGGG |
| | | SEIVLTQSPGTLSLSPGERATLSCRASQSVTSSYLAWYQ |
| | | QKPGQAPRLLINVGSRRATGIPDRFSGSGSGTDFTLTIS |
| | | RLEPEDFAVYYCQQGIMLPPTFGQGTKVEIK |
| 97 | CD28(mAb 9.3) VHCH | EVKLQQSGPGLVTPSQSLSITCTVSGFSLSDYGVHWVRQ |
| | “EE”-hu IgG1 Fc PGLALA | SPGQGLEWLGVIWAGGGTNYNSALMSRKSISKDNSKSQV |
| | FAP(4B9) VHCL | FLKMNSLQADDTAVYYCARDKGYSYYYSMDYWGQGTSVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPSRDELTKNQVSLTCLVKGFYPSDIAVEWESNGQP |
| | | ENNYKTTPPVLDSDGSFFLYSKLTVDKSRWQQGNVFSCS |
| | | VMHEALHNHYTQKSLSLSPGGGGGSGGGGSGGGGSGGGG |
| | | SEVQLLESGGGLVQPGGSLRLSCAASGFTFSSYAMSWVR |
| | | QAPGKGLEWVSAIIGSGASTYYADSVKGRFTISRDNSKN |
| | | TLYLQMNSLRAEDTAVYYCAKGWFGGFNYWGQGTLVTVS |
| | | SASVAAPSVFIFPPSDEQLKSGTASVVCLLNNFYPREAK |
| | | VQWKVDNALQSGNSQESVTEQDSKDSTYSLSSTLTLSKA |
| | | DYEKHKVYACEVTHQGLSSPVTKSFNRGEC |
| 98 | CD28(mAb 9.3) VLCL | DIELTQSPASLAVSLGQRATISCRASESVEYYVTSLMQW |
| | “RK” | YQQKPGQPPKLLIFAASNVESGVPARFSGSGSGTNFSLN |
| | | IHPVDEDDVAMYFCQQSRKVPYTFGGGTKLEIKRTVAAP |
| | | SVFIFPPSDRKLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 99 | FAP(4B9) VLCH1 | EIVLTQSPGTLSLSPGERATLSCRASQSVTSSYLAWYQQ |
| | | KPGQAPRLLINVGSRRATGIPDRFSGSGSGTDFTLTISR |
| | | LEPEDFAVYYCQQGIMLPPTFGQGTKVEIKSSASTKGPS |
| | | VFPLAPSSKSTSGGTAALGCLVKDYFPEPVTVSWNSGAL |
| | | TSGVHTFPAVLQSSGLYSLSSVVTVPSSSLGTQTYICNV |
| | | NHKPSNTKVDKKVEPKSC |
| 100 | CD28(mAb 9.3) VLCH1- | DIELTQSPASLAVSLGQRATISCRASESVEYYVTSLMQW |
| | FAP(4B9) VHCH1 “EE”- | YQQKPGQPPKLLIFAASNVESGVPARFSGSGSGTNFSLN |
| | hu IgG1 Fc knob PGLALA | IHPVDEDDVAMYFCQQSRKVPYTFGGGTKLEIKSSASTK |
| | | GPSVFPLAPSSKSTSGGTAALGCLVKDYFPEPVTVSWNS |
| | | GALTSGVHTFPAVLQSSGLYSLSSVVTVPSSSLGTQTYI |
| | | CNVNHKPSNTKVDKKVEPKSCDGGGGSGGGGSEVQLLES |
| | | GGGLVQPGGSLRLSCAASGETFSSYAMSWVRQAPGKGLE |
| | | WVSAIIGSGASTYYADSVKGRFTISRDNSKNTLYLQMNS |
| | | LRAEDTAVYYCAKGWFGGFNYWGQGTLVTVSSASTKGPS |
| | | VFPLAPSSKSTSGGTAALGCLVEDYFPEPVTVSWNSGAL |
| | | TSGVHTFPAVLQSSGLYSLSSVVTVPSSSLGTQTYICNV |
| | | NHKPSNTKVDEKVEPKSCDKTHTCPPCPAPEAAGGPSVF |
| | | LFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALGAPIEKTISKAKGQPREPQVYTLPPCRDEL |
| | | TKNQVSLWCLVKGFYPSDIAVEWESNGQPENNYKTTPPV |
| | | LDSDGSFFLYSKLTVDKSRWQQGNVFSCSVMHEALHNHY |
| | | TQKSLSLSP |
| 101 | CD28(mAb 9.3) VHCL | EVKLQQSGPGLVTPSQSLSITCTVSGFSLSDYGVHWVRQ |
| | | SPGQGLEWLGVIWAGGGTNYNSALMSRKSISKDNSKSQV |
| | | FLKMNSLQADDTAVYYCARDKGYSYYYSMDYWGQGTSVT |
| | | VSSASVAAPSVFIFPPSDEQLKSGTASVVCLLNNFYPRE |
| | | AKVQWKVDNALQSGNSQESVTEQDSKDSTYSLSSTLTLS |
| | | KADYEKHKVYACEVTHQGLSSPVTKSFNRGEC |
| 102 | CD28(mAb 9.3) VHCH1 | EVKLQQSGPGLVTPSQSLSITCTVSGFSLSDYGVHWVRQ |
| | “EE”-hu IgG1 Fc PGLALA | SPGQGLEWLGVIWAGGGTNYNSALMSRKSISKDNSKSQV |
| | CEA VHCL | FLKMNSLQADDTAVYYCARDKGYSYYYSMDYWGQGTSVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPSRDELTKNQVSLTCLVKGFYPSDIAVEWESNGQP |
| | | ENNYKTTPPVLDSDGSFFLYSKLTVDKSRWQQGNVFSCS |
| | | VMHEALHNHYTQKSLSLSPGGGGGSGGGGSGGGGSGGGG |
| | | SEVQLVESGGGLVQPGRSLRLSCAASGFTVSSYWMHWVR |
| | | QAPGKGLEWVGFIRNKANGGTTEYAASVKGRFTISRDDS |
| | | KNTLYLQMNSLRAEDTAVYYCARDRGLRFYFDYWGQGTT |
| | | VTVSSASVAAPSVFIFPPSDEQLKSGTASVVCLLNNFYP |
| | | REAKVQWKVDNALQSGNSQESVTEQDSKDSTYSLSSTLT |
| | | LSKADYEKHKVYACEVTHQGLSSPVTKSFNRGEC |
| 103 | CD28(mAb 9.3) VHCH1- | EVKLQQSGPGLVTPSQSLSITCTVSGFSLSDYGVHWVRQ |
| | hu IgG1 Fc knob CEA VH | SPGQGLEWLGVIWAGGGTNYNSALMSRKSISKDNSKSQV |
| | | FLKMNSLQADDTAVYYCARDKGYSYYYSMDYWGQGTSVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSPGGGGGSGGGGSGGGGSGGGG |
| | | SEVQLVESGGGLVQPGRSLRLSCAASGFTVSSYWMHWVR |
| | | QAPGKGLEWVGFIRNKANGGTTEYAASVKGRFTISRDDS |
| | | KNTLYLQMNSLRAEDTAVYYCARDRGLRFYFDYWGQGTT |
| | | VTVSS |
| 104 | CD28(mAb 9.3) VHCH1- | EVKLQQSGPGLVTPSQSLSITCTVSGFSLSDYGVHWVRQ |
| | hu IgG1 Fc hole CEA VL | SPGQGLEWLGVIWAGGGTNYNSALMSRKSISKDNSKSQV |
| | | FLKMNSLQADDTAVYYCARDKGYSYYYSMDYWGQGTSVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | CTLPPSRDELTKNQVSLSCAVKGFYPSDIAVEWESNGQP |
| | | ENNYKTTPPVLDSDGSFFLVSKLTVDKSRWQQGNVFSCS |
| | | VMHEALHNHYTQKSLSLSPGGGGGSGGGGSGGGGSGGGG |
| | | SQAVLTQPASLSASPGASASLTCTLRRGINVGAYSIYWY |
| | | QQKPGSPPQYLLRYKSDSDKQQGSGVSSRFSASKDASAN |
| | | AGILLISGLQSEDEADYYCMIWHSGASAVFGGGTKLTVL |
| 105 | CD28(mAb 9.3) VHCH1 | EVKLQQSGPGLVTPSQSLSITCTVSGFSLSDYGVHWVRQ |
| | “EE”-hu IgG1 Fc hole | SPGQGLEWLGVIWAGGGTNYNSALMSRKSISKDNSKSQV |
| | PGLALA HYRF | FLKMNSLQADDTAVYYCARDKGYSYYYSMDYWGQGTSVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | CTLPPSRDELTKNQVSLSCAVKGFYPSDIAVEWESNGQP |
| | | ENNYKTTPPVLDSDGSFFLVSKLTVDKSRWQQGNVFSCS |
| | | VMHEALHNHYTQKSLSLSP |
| 106 | CD28(mAb 9.3) VLCL | DIELTQSPASLAVSLGQRATISCRASESVEYYVTSLMQW |
| | “RK” | YQQKPGQPPKLLIFAASNVESGVPARFSGSGSGTNFSLN |
| | | IHPVDEDDVAMYFCQQSRKVPYTFGGGTKLEIKRTVAAP |
| | | SVFIFPPSDRKLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 107 | CD28(SA) VHCH1 “EE” hu | QVQLVQSGAEVKKPGASVKVSCKASGYTFTSYYIHWVRQ |
| | IgG1 Fc hole PGLALA | APGQGLEWIGCIYPGNVNTNYNEKFKDRATLTVDTSIST |
| | FAP(4B9) VH- | AYMELSRLRSDDTAVYFCTRSHYGLDWNFDVWGQGTTVT |
| | CEA(Medi-565) VHCL | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSPGGGGGSGGGGSGGGGSGGGG |
| | | SEVQLLESGGGLVQPGGSLRLSCAASGETFSSYAMSWVR |
| | | QAPGKGLEWVSAIIGSGASTYYADSVKGRFTISRDNSKN |
| | | TLYLQMNSLRAEDTAVYYCAKGWEGGENYWGQGTLVTVS |
| | | SGGGGSGGGGSGGGGSGGGGSEVQLVESGGGLVQPGRSL |
| | | RLSCAASGFTVSSYWMHWVRQAPGKGLEWVGFIRNKANG |
| | | GTTEYAASVKGRFTISRDDSKNTLYLQMNSLRAEDTAVY |
| | | YCARDRGLRFYFDYWGQGTTVTVSSASVAAPSVFIFPPS |
| | | DEQLKSGTASVVCLLNNFYPREAKVQWKVDNALQSGNSQ |
| | | ESVTEQDSKDSTYSLSSTLTLSKADYEKHKVYACEVTHQ |
| | | GLSSPVTKSFNRGEC |
| 108 | CD28(SA) VHCH1 “EE” hu | QVQLVQSGAEVKKPGASVKVSCKASGYTFTSYYIHWVRQ |
| | IgG1 Fc knob PGLALA | APGQGLEWIGCIYPGNVNTNYNEKFKDRATLTVDTSIST |
| | FAP(4B9) VL | AYMELSRLRSDDTAVYFCTRSHYGLDWNFDVWGQGTTVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | CTLPPSRDELTKNQVSLSCAVKGFYPSDIAVEWESNGQP |
| | | ENNYKTTPPVLDSDGSFFLVSKLTVDKSRWQQGNVFSCS |
| | | VMHEALHNHYTQKSLSLSPGGGGGSGGGGSGGGGSGGGG |
| | | SEIVLTQSPGTLSLSPGERATLSCRASQSVTSSYLAWYQ |
| | | QKPGQAPRLLINVGSRRATGIPDRFSGSGSGTDFTLTIS |
| | | RLEPEDFAVYYCQQGIMLPPTFGQGTKVEIK |
| 109 | CEA VLCH1 | QAVLTQPASLSASPGASASLTCTLRRGINVGAYSIYWYQ |
| | | QKPGSPPQYLLRYKSDSDKQQGSGVSSRFSASKDASANA |
| | | GILLISGLQSEDEADYYCMIWHSGASAVFGGGTKLTVLS |
| | | SASTKGPSVFPLAPSSKSTSGGTAALGCLVKDYFPEPVT |
| | | VSWNSGALTSGVHTFPAVLQSSGLYSLSSVVTVPSSSLG |
| | | TQTYICNVNHKPSNTKVDKKVEPKSC |
| 110 | CD28(SA) VHCH1 hu IgG1 | QVQLVQSGAEVKKPGASVKVSCKASGYTFTSYYIHWVRQ |
| | Fc hole PGLALA FAP(4B9) | APGQGLEWIGCIYPGNVNTNYNEKFKDRATLTVDTSIST |
| | VH-CEA VH | AYMELSRLRSDDTAVYFCTRSHYGLDWNFDVWGQGTTVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSPGGGGGSGGGGSGGGGSGGGG |
| | | SEVQLLESGGGLVQPGGSLRLSCAASGETFSSYAMSWVR |
| | | QAPGKGLEWVSAIIGSGASTYYADSVKGRFTISRDNSKN |
| | | TLYLQMNSLRAEDTAVYYCAKGWEGGENYWGQGTLVTVS |
| | | SGGGGSGGGGSGGGGSGGGGSEVQLVESGGGLVQPGRSL |
| | | RLSCAASGFTVSSYWMHWVRQAPGKGLEWVGFIRNKANG |
| | | GTTEYAASVKGRFTISRDDSKNTLYLQMNSLRAEDTAVY |
| | | YCARDRGLRFYFDYWGQGTTVTVSS |
| 111 | CD28(SA) VHCH1 Fc knob | QVQLVQSGAEVKKPGASVKVSCKASGYTFTSYYIHWVRQ |
| | PGLALA FAP(4B9) VL- | APGQGLEWIGCIYPGNVNTNYNEKFKDRATLTVDTSIST |
| | CEA VL | AYMELSRLRSDDTAVYFCTRSHYGLDWNFDVWGQGTTVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | CTLPPSRDELTKNQVSLSCAVKGFYPSDIAVEWESNGQP |
| | | ENNYKTTPPVLDSDGSFFLVSKLTVDKSRWQQGNVFSCS |
| | | VMHEALHNHYTQKSLSLSPGGGGGSGGGGSGGGGSGGGG |
| | | SEIVLTQSPGTLSLSPGERATLSCRASQSVTSSYLAWYQ |
| | | QKPGQAPRLLINVGSRRATGIPDRFSGSGSGTDFTLTIS |
| | | RLEPEDFAVYYCQQGIMLPPTFGQGTKVEIKGGGGSGGG |
| | | GSGGGGSGGGGSQAVLTQPASLSASPGASASLTCTLRRG |
| | | INVGAYSIYWYQQKPGSPPQYLLRYKSDSDKQQGSGVSS |
| | | RFSASKDASANAGILLISGLQSEDEADYYCMIWHSGASA |
| | | VFGGGTKLTVL |
| 112 | VH (CD28 parental) CH1- | QVQLVQSGAEVKKPGASVKVSCKASGYTFTSYYIHWVRQ |
| | hu IgG1 Fc knob PGLALA | APGQGLEWIGCIYPGNVNTNYNEKFKDRATLTVDTSIST |
| | | AYMELSRLRSDDTAVYFCTRSHYGLDWNFDVWGQGTTVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSP |
| 113 | VH (CD28 variant g) CH1- | QVQLVQSGAEVKKPGASVKVSCKASGYTFTSYYIHWVRQ |
| | hu IgG1 Fc knob PGLALA | APGQGLEWIGSIYPRNVQTNYNEKFKDRATLTVDTSIST |
| | | AYMELSRLRSDDTAVYFCTRSHYGLDHNFDVWGQGTTVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSP |
| 114 | VH (CD28 variant f) CH1- | QVQLVQSGAEVKKPGASVKVSCKASGYTFTSYYIHWVRQ |
| | hu IgG1 Fc knob PGLALA | APGQGLEWIGSIYPGNVQTNYNEKFKDRATLTVDTSIST |
| | | AYMELSRLRSDDTAVYFCTRSHYGLDFNFDVWGQGTTVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSP |
| 115 | VH (CD28 variant j) CH1- | EVQLVESGGGLVQPGGSLRLSCAASGETFTSYYIHWVRQ |
| | hu IgG1 Fc knob PGLALA | APGKGLEWVASIYPGNVATRYADSVKGRFTISADTSKNT |
| | | AYLQMNSLRAEDTAVYYCTRSHYGLDWNFDVWGQGTTVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSP |
| 116 | VH (CD28 variant e) CH1- | QVQLVQSGAEVKKPGASVKVSCKASGYTFTSYYIHWVRQ |
| | hu IgG1 Fc knob PGLALA | APGQGLEWIGSIYPGNVQTNYNEKFKDRATLTVDTSIST |
| | | AYMELSRLRSDDTAVYFCTRSHYGLDWNFDVWGQGTTVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSP |
| 117 | VH (CD28 variant b) CH1- | QVQLVQSGAEVKKPGASVKVSCKASGYTFTSYYIHWVRQ |
| | hu IgG1 Fc knob PGLALA | APGQGLEWIGSIYPGNVQTNYNEKFKDRATLTVDTSIST |
| | | AYMELSRLRSDDTAVYFCTRSHYGLDHNFDVWGQGTTVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSP |
| 118 | VH (CD28 variant a) CH1- | QVQLVQSGAEVKKPGASVKVSCKASGYTFTSYYIHWVRQ |
| | hu IgG1 Fc knob PGLALA | APGQGLEWIGSIYPGNVNTNYNEKFKDRATLTVDTSIST |
| | | AYMELSRLRSDDTAVYFCTRSHYGLDWNFDVWGQGTTVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSP |
| 119 | VH (CD28 variant i) CH1- | EVQLVESGGGLVQPGGSLRLSCAASGETFTSYYIHWVRQ |
| | hu IgG1 Fc knob PGLALA | APGKGLEWVASIYPGNVNTRYADSVKGRFTISADTSKNT |
| | | AYLQMNSLRAEDTAVYYCTRSHYGLDWNFDVWGQGTTVT |
| | | VSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | YTLPPCRDELTKNQVSLWCLVKGFYPSDIAVEWESNGQP |
| | | ENNYKTTPPVLDSDGSFFLYSKLTVDKSRWQQGNVFSCS |
| | | VMHEALHNHYTQKSLSLSP |
| 120 | VL (CD28 variant k)-CL | DIQMTQSPSSLSASVGDRVTITCHASQNIYVHLNWYQQK |
| | | PGKAPKLLIYKASNLHTGVPSRFSGSGSGTDFTLTISSL |
| | | QPEDFATYYCQQAQTYPYTFGGGTKVEIKRTVAAPSVFI |
| | | FPPSDRKLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 121 | VL (CD28 variant 1)-CL | DIQMTQSPSSLSASVGDRVTITCHASQNIYVFLNWYQQK |
| | | PGKAPKLLIYKASNLHTGVPSRFSGSGSGTDFTLTISSL |
| | | QPEDFATYYCQQGQTYPYTFGGGTKVEIKRTVAAPSVFI |
| | | FPPSDRKLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 122 | VL (CD28 variant m)-CL | DIQMTQSPSSLSASVGDRVTITCHASQNIYVYLNWYQQK |
| | | PGKAPKLLIYKASNLHTGVPSRFSGSGSGTDFTLTISSL |
| | | QPEDFATYYCQQGQTYPYTFGGGTKVEIKRTVAAPSVFI |
| | | FPPSDRKLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 123 | VL (CD28 variant r)-CL | DIQMTQSPSSLSASVGDRVTITCHASQGIYVYLNWYQQK |
| | | PGKAPKLLIYKASNLHTGVPSRFSGSGSGTDFTLTISSL |
| | | QPEDFATYYCQQGQTYPYTFGGGTKVEIKRTVAAPSVFI |
| | | FPPSDRKLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 124 | VL (CD28 variant s)-CL | DIQMTQSPSSLSASVGDRVTITCHASQGISVYLNWYQQK |
| | | PGKAPKLLIYKASNLHTGVPSRFSGSGSGTDFTLTISSL |
| | | QPEDFATYYCQQGQTYPYTFGGGTKVEIKRTVAAPSVFI |
| | | FPPSDRKLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 125 | VL (CD28 variant t)-CL | DIQMTQSPSSLSASVGDRVTITCRASQNIYVWLNWYQQK |
| | | PGKAPKLLIYKASNLYSGVPSRFSGSRSGTDFTLTISSL |
| | | QPEDFATYYCQQGQTYPYTFGQGTKLEIKRTVAAPSVFI |
| | | FPPSDRKLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 126 | hu IgG1 Fc hole PGLALA, | DKTHTCPPCPAPEAAGGPSVFLFPPKPKDTLMISRTPEV |
| | HYRF | TCVVVDVSHEDPEVKFNWYVDGVEVHNAKTKPREEQYNS |
| | | TYRVVSVLTVLHQDWLNGKEYKCKVSNKALGAPIEKTIS |
| | | KAKGQPREPQVCTLPPSRDELTKNQVSLSCAVKGFYPSD |
| | | IAVEWESNGQPENNYKTTPPVLDSDGSFFLVSKLTVDKS |
| | | RWQQGNVFSCSVMHEALHNRFTQKSLSLSP |
| 127 | CEA CDR-H1 | SYWMH |
| 128 | CEA CDR-H2 | FIRNKANGGTTEYAASVKG |
| 129 | CEA CDR-H3 | DRGLRFYFDY |
| 130 | CEA CDR-L1 | TLRRGINVGAYSTY |
| 131 | CEA CDR-L2 | YKSDSDKQQGSGV |
| 132 | CEA CDR-L3 | MIWHSGASAV |
| 133 | CEA VH | EVQLVESGGGLVQPGRSLRLSCAASGFTVSSYWMHWVRQ |
| | | APGKGLEWVGFIRNKANGGTTEYAASVKGRFTISRDDSK |
| | | NTLYLQMNSLRAEDTAVYYCARDRGLRFYFDYWGQGTTV |
| | | TVSS |
| 134 | CEA VL | QAVLTQPASLSASPGASASLTCTLRRGINVGAYSIYWYQ |
| | | QKPGSPPQYLLRYKSDSDKQQGSGVSSRFSASKDASANA |
| | | GILLISGLQSEDEADYYCMIWHSGASAVEGGGTKLTVL |
| 135 | His-tagged human FAP | RPSRVHNSEENTMRALTLKDILNGTFSYKTFFPNWISGQ |
| | RAD | EYLHQSADNNIVLYNIETGQSYTILSNRTMKSVNASNYG |
| | | LSPDRQFVYLESDYSKLWRYSYTATYYIYDLSNGEFVRG |
| | | NELPRPIQYLCWSPVGSKLAYVYQNNIYLKQRPGDPPFQ |
| | | ITFNGRENKIFNGIPDWVYEEEMLATKYALWWSPNGKFL |
| | | AYAEFNDTDIPVIAYSYYGDEQYPRTINIPYPKAGAKNP |
| | | VVRIFIIDTTYPAYVGPQEVPVPAMIASSDYYFSWLTWV |
| | | TDERVCLQWLKRVQNVSVLSICDFREDWQTWDCPKTQEH |
| | | IEESRTGWAGGFFVSTPVFSYDAISYYKIFSDKDGYKHI |
| | | HYIKDTVENAIQITSGKWEAINIFRVTQDSLFYSSNEFE |
| | | EYPGRRNIYRISIGSYPPSKKCVTCHLRKERCQYYTASF |
| | | SDYAKYYALVCYGPGIPISTLHDGRTDQEIKILEENKEL |
| | | ENALKNIQLPKEEIKKLEVDEITLWYKMILPPQFDRSKK |
| | | YPLLIQVYGGPCSQSVRSVFAVNWISYLASKEGMVIALV |
| | | DGRGTAFQGDKLLYAVYRKLGVYEVEDQITAVRKFIEMG |
| | | FIDEKRIAIWGWSYGGYVSSLALASGTGLFKCGIAVAPV |
| | | SSWEYYASVYTERFMGLPTKDDNLEHYKNSTVMARAEYF |
| | | RNVDYLLIHGTADDNVHFQNSAQIAKALVNAQVDFQAMW |
| | | YSDQNHGLSGLSTNHLYTHMTHFLKQCFSLSDGKKKKKK |
| | | GHHHHHH |
| 136 | mouse FAP | UniProt accession no. P97321 |
| 137 | His-tagged mouse FAP ECD | RPSRVYKPEGNTKRALTLKDILNGTFSYKTYFPNWISEQ |
| | | EYLHQSEDDNIVFYNIETRESYIILSNSTMKSVNATDYG |
| | | LSPDRQFVYLESDYSKLWRYSYTATYYIYDLQNGEFVRG |
| | | YELPRPIQYLCWSPVGSKLAYVYQNNIYLKQRPGDPPFQ |
| | | ITYTGRENRIFNGIPDWVYEEEMLATKYALWWSPDGKFL |
| | | AYVEFNDSDIPIIAYSYYGDGQYPRTINIPYPKAGAKNP |
| | | VVRVFIVDTTYPHHVGPMEVPVPEMIASSDYYFSWLTWV |
| | | SSERVCLQWLKRVQNVSVLSICDFREDWHAWECPKNQEH |
| | | VEESRTGWAGGFEVSTPAFSQDATSYYKIFSDKDGYKHI |
| | | HYIKDTVENAIQITSGKWEAIYIFRVTQDSLFYSSNEFE |
| | | GYPGRRNIYRISIGNSPPSKKCVTCHLRKERCQYYTASF |
| | | SYKAKYYALVCYGPGLPISTLHDGRTDQEIQVLEENKEL |
| | | ENSLRNIQLPKVEIKKLKDGGLTFWYKMILPPQFDRSKK |
| | | YPLLIQVYGGPCSQSVKSVFAVNWITYLASKEGIVIALV |
| | | DGRGTAFQGDKFLHAVYRKLGVYEVEDQLTAVRKFIEMG |
| | | FIDEERIAIWGWSYGGYVSSLALASGTGLFKCGIAVAPV |
| | | SSWEYYASIYSERFMGLPTKDDNLEHYKNSTVMARAEYF |
| | | RNVDYLLIHGTADDNVHFQNSAQIAKALVNAQVDFQAMW |
| | | YSDQNHGILSGRSQNHLYTHMTHFLKQCFSLSDGKKKKK |
| | | KGHHHHHH |
| 138 | His-tagged cynomolgus FAP | RPPRVHNSEENTMRALTLKDILNGTFSYKTFFPNWISGQ |
| | ECD | EYLHQSADNNIVLYNIETGQSYTILSNRTMKSVNASNYG |
| | | LSPDRQFVYLESDYSKLWRYSYTATYYIYDLSNGEFVRG |
| | | NELPRPIQYLCWSPVGSKLAYVYQNNIYLKQRPGDPPFQ |
| | | ITFNGRENKIFNGIPDWVYEEEMLATKYALWWSPNGKFL |
| | | AYAEFNDTDIPVIAYSYYGDEQYPRTINIPYPKAGAKNP |
| | | FVRIFIIDTTYPAYVGPQEVPVPAMIASSDYYFSWLTWV |
| | | TDERVCLQWLKRVQNVSVLSICDFREDWQTWDCPKTQEH |
| | | IEESRTGWAGGFFVSTPVFSYDAISYYKIFSDKDGYKHI |
| | | HYIKDTVENAIQITSGKWEAINIFRVTQDSLFYSSNEFE |
| | | DYPGRRNIYRISIGSYPPSKKCVTCHLRKERCQYYTASF |
| | | SDYAKYYALVCYGPGIPISTLHDGRTDQEIKILEENKEL |
| | | ENALKNIQLPKEEIKKLEVDEITLWYKMILPPQFDRSKK |
| | | YPLLIQVYGGPCSQSVRSVFAVNWISYLASKEGMVIALV |
| | | DGRGTAFQGDKLLYAVYRKLGVYEVEDQITAVRKFIEMG |
| | | FIDEKRIAIWGWSYGGYVSSLALASGTGLFKCGIAVAPV |
| | | SSWEYYASVYTERFMGLPTKDDNLEHYKNSTVMARAEYF |
| | | RNVDYLLIHGTADDNVHFQNSAQIAKALVNAQVDFQAMW |
| | | YSDQNHGLSGLSTNHLYTHMTHFLKQCFSLSDGKKKKKK |
| | | GHHHHHH |
| 139 | human FolR1 | UniProt accession no. P15328 |
| 140 | murine FolR1 | UniProt accession no. P35846 |
| 141 | cynomolgus FolR1 | UniProt accession no. G7PR14 |
| 142 | human MCSP | UniProt accession no. Q6UVK1 |
| 143 | human EGFR | UniProt accession no. P00533 |
| 144 | human HER2 | Uniprot accession no. P04626 |
| 145 | p95 HER2 | MPIWKFPDEEGACQPCPINCTHSCVDLDDKGCPAEQRAS |
| | | PLTSIISAVVGILLVVVLGVVFGILIKRRQQKIRKYTMR |
| | | RLLQETELVEPLTPSGAMPNQAQMRILKETELRKVKVLG |
| | | SGAFGTVYKGIWIPDGENVKIPVAIKVLRENTSPKANKE |
| | | ILDEAYVMAGVGSPYVSRLLGICLTSTVQLVTQLMPYGC |
| | | LLDHVRENRGRLGSQDLLNWCMQIAKGMSYLEDVRLVHR |
| | | DLAARNVLVKSPNHVKITDFGLARLLDIDETEYHADGGK |
| | | VPIKWMALESILRRRFTHQSDVWSYGVTVWELMTFGAKP |
| | | YDGIPAREIPDLLEKGERLPQPPICTIDVYMIMVKCWMI |
| | | DSECRPRFRELVSEFSRMARDPQRFVVIQNEDLGPASPL |
| | | DSTFYRSLLEDDDMGDLVDAEEYLVPQQGFFCPDPAPGA |
| | | GGMVHHRHRSSSTRSGGGDLTLGLEPSEEEAPRSPLAPS |
| | | EGAGSDVFDGDLGMGAAKGLQSLPTHDPSPLQRYSEDPT |
| | | VPLPSETDGYVAPLTCSPQPEYVNQPDVRPQPPSPREGP |
| | | LPAARPAGATLERPKTLSPGKNGVVKDVFAFGGAVENPE |
| | | YLTPQGGAAPQPHPPPAFSPAFDNLYYWDQDPPERGAPP |
| | | STFKGTPTAENPEYLGLDVPV |
| 146 | Peptide linker (G4S) | GGGGS |
| 147 | Peptide linker (G4S)2 | GGGGSGGGGS |
| 148 | Peptide linker (SG4)2 | SGGGGSGGGG |
| 149 | Peptide linker G4(SG4)2 | GGGGSGGGGSGGGG |
| 150 | peptide linker | GSPGSSSSGS |
| 151 | (G4S)3 peptide linker | GGGGSGGGGSGGGGS3 |
| 152 | (G4S)4 peptide linker | GGGGSGGGGSGGGGSGGGGS |
| 153 | peptide linker | GSGSGSGS |
| 154 | peptide linker | GSGSGNGS |
| 155 | peptide linker | GGSGSGSG |
| 156 | peptide linker | GGSGSG |
| 157 | peptide linker | GGSG |
| 158 | peptide linker | GGSGNGSG |
| 159 | peptide linker | GGNGSGSG |
| 160 | peptide linker | GGNGSG |
| 161 | CEACAM5-based antigen | QLTTESMPFNVAEGKEVLLLVHNLPQQLFGYSWYKGERV |
| | Hu N(A2-B2)A-avi-His | DGNRQIVGYAIGTQQATPGPANSGRETIYPNASLLIQNV |
| | | TQNDTGFYTLQVIKSDLVNEEATGQFHVYPELPKPFITS |
| | | NNSNPVEDEDAVALTCEPEIQNTTYLWWVNNQSLPVSPR |
| | | LQLSNDNRTLTLLSVTRNDVGPYECGIQNKLSVDHSDPV |
| | | ILNVLYGPDDPTISPSYTYYRPGVNLSLSCHAASNPPAQ |
| | | YSWLIDGNIQQHTQELFISNITEKNSGLYTCQANNSASG |
| | | HSRTTVKTITVSALSPVVAKPQIKASKTTVTGDKDSVNL |
| | | TCSINDTGISIRWEEKNQSLPSSERMKLSQGNITLSINP |
| | | VKREDAGTYWCEVFNPISKNQSDPIMLNVNYNALPQENL |
| | | INVDGSGLNDIFEAQKIEWHEARAHHHHHH |
| 162 | Avi-tag | GLNDIFEAQKIEWHE |

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991). Amino acids of antibody chains are numbered and referred to according to the numbering systems according to Kabat (Kabat, E. A., et al., Sequences of Proteins of Immunological Interest, 5th ed., Public Health Service, National Institutes of Health, Bethesda, Md. (1991)) as defined above.

The following numbered paragraphs (paras) describe aspects of the present invention:

1. A superagonistic CD28 antigen binding molecule, which is capable of bivalent binding to CD28 and comprises

(a) two or more antigen binding domains capable of specific binding to CD28,
(b) at least one antigen binding domain capable of specific binding to a tumor-associated antigen, and
(c) an Fc domain composed of a first and a second subunit capable of stable association comprising one or more amino acid substitution that reduces the binding affinity of the antigen binding molecule to an Fc receptor and/or effector function.

2. The superagonistic CD28 antigen binding molecule of para 1, comprising two antigen binding domains capable of specific binding to CD28.

3. The superagonistic CD28 antigen binding molecule of paras 1 or 2, wherein the Fc domain is an IgG, particularly an IgG1 Fc domain or an IgG4 Fc domain.

4. The superagonistic CD28 antigen binding molecule of any one of paras 1 to 3, wherein the Fc domain is of human IgG1 subclass and comprises the amino acid mutations L234A, L235A and P329G (numbering according to Kabat EU index).

5. The superagonistic CD28 antigen binding molecule of any one of paras 1 to 4, wherein each of the antigen binding domains capable of specific binding to CD28 comprises

(i) a heavy chain variable region (V_{H}CD28) comprising a heavy chain complementary determining region CDR-H1 of SEQ ID NO: 20, a CDR-H2 of SEQ ID NO: 21, and a CDR-H3 of SEQ ID NO: 22, and a light chain variable region (V_{L}CD28) comprising a light chain complementary determining region CDR-L1 of SEQ ID NO: 23, a CDR-L2 of SEQ ID NO: 24 and a CDR-L3 of SEQ ID NO: 25; or
(ii) a heavy chain variable region (V_{H}CD28) comprising a CDR-H1 of SEQ ID NO: 36, a CDR-H2 of SEQ ID NO: 37, and a CDR-H3 of SEQ ID NO: 38, and a light chain variable region (V_{L}CD28) comprising a CDR-L1 of SEQ ID NO: 39, a CDR-L2 of SEQ ID NO: 40 and a CDR-L3 of SEQ ID NO: 41.

6. The superagonistic CD28 antigen binding molecule of any one of paras 1 to 5, wherein each of the antigen binding domains capable of specific binding to CD28 comprises a heavy chain variable region (V_{H}CD28) comprising a CDR-H1 of SEQ ID NO: 20, a CDR-H2 of SEQ ID NO: 21, and a CDR-H3 of SEQ ID NO: 22, and a light chain variable region (V_{L}CD28) comprising a CDR-L1 of SEQ ID NO: 23, a CDR-L2 of SEQ ID NO: 24 and a CDR-L3 of SEQ ID NO: 25.

7. The superagonistic CD28 antigen binding molecule of any one of paras 1 to 5, wherein each of the antigen binding domains capable of specific binding to CD28 comprises a heavy chain variable region (V_{H}CD28) comprising an amino acid sequence that is at least about 95%, 96%, 97%, 98%, 99% or 100% identical to the amino acid sequence of SEQ ID NO:26, and a light chain variable region (V_{L}CD28) comprising an amino acid sequence that is at least about 95%, 96%, 97%, 98%, 99% or 100% identical to the amino acid sequence of SEQ ID NO:27.

8. The superagonistic CD28 antigen binding molecule of any one of paras 1 to 5, wherein each of the antigen binding domains capable of specific binding to CD28 comprises a heavy chain variable region (V_{H}CD28) comprising an amino acid sequence selected from the group consisting of SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50 and SEQ ID NO:51, and a light chain variable region (V_{L}CD28) comprising an amino acid sequence selected from the group consisting of SEQ ID NO:27, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60 and SEQ ID NO:60.

9. The superagonistic CD28 antigen binding molecule of any one of paras 1 to 5 or 8, wherein each of the antigen binding domains capable of specific binding to CD28 comprises

(a) a heavy chain variable region (V_{H}CD28) comprising the amino acid sequence of SEQ ID NO:47 and a light chain variable region (V_{L}CD28) comprising the amino acid sequence of SEQ ID NO:54, or

(b) a heavy chain variable region (V_{H}CD28) comprising the amino acid sequence of SEQ ID NO:47 and a light chain variable region (V_{L}CD28) comprising the amino acid sequence of SEQ ID NO:27, or

(c) a heavy chain variable region (V_{H}CD28) comprising the amino acid sequence of SEQ ID NO:51 and a light chain variable region (V_{L}CD28) comprising the amino acid sequence of SEQ ID NO:61, or

(d) a heavy chain variable region (V_{H}CD28) comprising the amino acid sequence of SEQ ID NO:46 and a light chain variable region (V_{L}CD28) comprising the amino acid sequence of SEQ ID NO:53, or

(e) a heavy chain variable region (V_{H}CD28) comprising the amino acid sequence of SEQ ID NO:46 and a light chain variable region (V_{L}CD28) comprising the amino acid sequence of SEQ ID NO:54, or

(f) a heavy chain variable region (V_{H}CD28) comprising the amino acid sequence of SEQ ID NO:46 and a light chain variable region (V_{L}CD28) comprising the amino acid sequence of SEQ ID NO:59, or

(g) a heavy chain variable region (V_{H}CD28) comprising the amino acid sequence of SEQ ID NO:46 and a light chain variable region (V_{L}CD28) comprising the amino acid sequence of SEQ ID NO:27, or

(h) a heavy chain variable region (V_{H}CD28) comprising the amino acid sequence of SEQ ID NO:43 and a light chain variable region (V_{L}CD28) comprising the amino acid sequence of SEQ ID NO:27, or

(i) a heavy chain variable region (V_{H}CD28) comprising the amino acid sequence of SEQ ID NO:42 and a light chain variable region (V_{L}CD28) comprising the amino acid sequence of SEQ ID NO:53, or

(j) a heavy chain variable region (V_{H}CD28) comprising the amino acid sequence of SEQ ID NO:42 and a light chain variable region (V_{L}CD28) comprising the amino acid sequence of SEQ ID NO:59, or

(k) a heavy chain variable region (V_{H}CD28) comprising the amino acid sequence of SEQ ID NO:42 and a light chain variable region (V_{L}CD28) comprising the amino acid sequence of SEQ ID NO:27.

10. The superagonistic CD28 antigen binding molecule of any one of paras 1 to 9, wherein each of the antigen binding domains capable of specific binding to CD28 is a Fab fragment.

11. The superagonistic CD28 antigen binding molecule of any one of paras 1 to 10, wherein the antigen binding domain capable of specific binding to a tumor-associated antigen is an antigen binding domain capable of specific binding to Carcinoembryonic Antigen (CEA).

12. The superagonistic CD28 antigen binding molecule of any one of paras 1 to 12, wherein the antigen binding domain capable of specific binding to CEA comprises a heavy chain variable region (V_{H}CEA) comprising (i) CDR-H1 comprising the amino acid sequence of SEQ ID NO:127, (ii) CDR-H2 comprising the amino acid sequence of SEQ ID NO:128, and (iii) CDR-H3 comprising the amino acid sequence of SEQ ID NO:129, and a light chain variable region (V_{L}CEA) comprising (iv) CDR-L1 comprising the amino acid sequence of SEQ ID NO:130, (v) CDR-L2 comprising the amino acid sequence of SEQ ID NO:131, and (vi) CDR-L3 comprising the amino acid sequence of SEQ ID NO:132.

13. The superagonistic CD28 antigen binding molecule of any one of paras 1 to 12, wherein the antigen binding domain capable of specific binding to CEA comprises a heavy chain variable region (V_{H}CEA) comprising an amino acid sequence that is at least about 95%, 96%, 97%, 98%, 99% or 100% identical to the amino acid sequence of SEQ ID NO:133, and a light chain variable region (V_{L}CEA) comprising an amino acid sequence that is at least about 95%, 96%, 97%, 98%, 99% or 100% identical to the amino acid sequence of SEQ ID NO:134.

14. The superagonistic CD28 antigen binding molecule of any one of paras 1 to 10, wherein the antigen binding domain capable of specific binding to a tumor-associated antigen is an antigen binding domain capable of specific binding to Fibroblast Activation Protein (FAP).

15. The superagonistic CD28 antigen binding molecule of any one of paras 1 to 10 or 14, wherein the antigen binding domain capable of specific binding to FAP comprises

(a) a heavy chain variable region (V_{H}FAP) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}FAP) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17, or

(b) a heavy chain variable region (V_{H}FAP) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}FAP) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9.

16. The superagonistic CD28 antigen binding molecule of any one of paras 1 to 10 or 14 or 15, wherein the antigen binding domain capable of specific binding to FAP comprises (a) a heavy chain variable region (V_{H}FAP) comprising an amino acid sequence that is at least about 95%, 96%, 97%, 98%, 99% or 100% identical to the amino acid sequence of SEQ ID NO:18, and a light chain variable region (V_{L}FAP) comprising an amino acid sequence that is at least about 95%, 96%, 97%, 98%, 99% or 100% identical to the amino acid sequence of SEQ ID NO:19, or

(b) a heavy chain variable region (V_{H}FAP) comprising an amino acid sequence that is at least about 95%, 96%, 97%, 98%, 99% or 100% identical to the amino acid sequence of SEQ ID NO:10, and a light chain variable region (V_{L}FAP) comprising an amino acid sequence that is at least about 95%, 96%, 97%, 98%, 99% or 100% identical to the amino acid sequence of SEQ ID NO:11.

17. The superagonistic CD28 antigen binding molecule of any one of paras 1 to 10 or 14 to 16, wherein the antigen binding domain capable of specific binding to FAP comprises a heavy chain variable region (V_{H}FAP) comprising the amino acid sequence of SEQ ID NO:18 and a light chain variable region (V_{L}FAP) comprising the amino acid sequence of SEQ ID NO:19.

18. The superagonistic CD28 antigen binding molecule of any one of paras 1 to 17, comprising

(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and

(b) a VH and VL domain capable of specific binding to a tumor-associated antigen, wherein the VH domain is connected via a peptide linker to the C-terminus of one of the two heavy chains and wherein the VL domain is connected via a peptide linker to the C-terminus of the second heavy chain.

19. The superagonistic CD28 antigen binding molecule of any one of paras 1 to 17, comprising

(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and

(b) a crossFab fragment capable of specific binding to a tumor-associated antigen which is connected via a peptide linker to the C-terminus of one of the two heavy chains.

20. The superagonistic CD28 antigen binding molecule of any one of paras 1 to 17, comprising

(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and

(b) two crossFab fragments capable of specific binding to a tumor-associated antigen, wherein one crossFab fragment is connected via a peptide linker to the C-terminus of one of the two heavy chains and wherein the other crossFab fragment is connected via a peptide linker to the C-terminus of the second heavy chain.

21. A polynucleotide encoding the bispecific antigen binding molecule of any one of paras 1 to 20.

22. A host cell comprising the polynucleotide of para 21.

23. A method of producing the superagonistic CD28 antigen binding molecule of any one of paras 1 to 20 comprising culturing the host cell of para 22 under conditions suitable for the expression of the bispecific antigen binding molecule.

24. A pharmaceutical composition comprising superagonistic CD28 antigen binding molecule of any one of paras 1 to 20 and at least one pharmaceutically acceptable excipient.

25. The pharmaceutical composition of para 24 for use in the treatment of cancer.

26. The superagonistic CD28 antigen binding molecule of any one of paras 1 to 20, or the pharmaceutical composition of para 24, for use as a medicament.

27. The superagonistic CD28 antigen binding molecule of any one of paras 1 to 20, or the pharmaceutical composition of para 24, for use in the treatment of cancer.

28. The superagonistic CD28 antigen binding molecule of any one of paras 1 to 20 for use in the treatment of cancer, wherein the superagonistic CD28 antigen binding molecule is administered in combination with a chemotherapeutic agent, radiation therapy and/or other agents for use in cancer immunotherapy.

29. Use of the superagonistic CD28 antigen binding molecule of any one of paras 1 to 20, or the pharmaceutical composition of para 24, in the manufacture of a medicament for the treatment of cancer.

30. A method of inhibiting the growth of tumor cells in an individual comprising administering to the individual an effective amount of the superagonistic CD28 antigen binding molecule of any one of paras 1 to 20, or the pharmaceutical composition of para 24, to inhibit the growth of the tumor cells.

31. A method of treating cancer comprising administering to the individual a therapeutically effective amount of the superagonistic CD28 antigen binding molecule of any one of claims 1 to 20, or the pharmaceutical composition of claim 24.

### EXAMPLES

The following are examples of methods and compositions of the invention. It is understood that various other embodiments may be practiced, given the general description provided above.

Recombinant DNA Techniques

Standard methods were used to manipulate DNA as described in Sambrook et al., Molecular cloning: A laboratory manual; Cold Spring Harbor Laboratory Press, Cold Spring Harbor, N.Y., 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

DNA Sequencing

DNA sequences were determined by double strand sequencing.

Gene Synthesis

Desired gene segments, where required, were either generated by PCR using appropriate templates or were synthesized at Geneart AG (Regensburg, Germany) or Genscript (New Jersey, USA) from synthetic oligonucleotides and PCR products by automated gene synthesis. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow subcloning into the respective expression vectors. All constructs were designed with a 5′-end DNA sequence coding for a leader peptide which targets proteins for secretion in eukaryotic cells.

Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE, size exclusion chromatography (SEC) or mass spectrometry.

SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2×PBS on a Dionex HPLC-System. The eluted protein was quantified by UV absorbance and integration of peak areas. BioRad Gel Filtration Standard 151-1901 served as a standard.

Mass Spectrometry

This section describes the characterization of the multispecific antibodies with VH/VL exchange (VH/VL CrossMabs) with emphasis on their correct assembly. The expected primary structures were analyzed by electrospray ionization mass spectrometry (ESI-MS) of the deglycosylated intact CrossMabs and deglycosylated/plasmin digested or alternatively deglycosylated/limited LysC digested CrossMabs.

The VH/VL CrossMabs were deglycosylated with N-Glycosidase F in a phosphate or Tris buffer at 37° C. for up to 17 h at a protein concentration of 1 mg/ml. The plasmin or limited LysC (Roche) digestions were performed with 100 μg deglycosylated VH/VL CrossMabs in a Tris buffer pH 8 at room temperature for 120 hours and at 37° C. for 40 min, respectively. Prior to mass spectrometry the samples were desalted via HPLC on a Sephadex G25 column (GE Healthcare). The total mass was determined via ESI-MS on a maXis 4G UHR-QTOF MS system (Bruker Daltonik) equipped with a TriVersa NanoMate source (Advion).

Determination of Binding and Binding Affinity of Multispecific Antibodies to the Respective Antigens Using Surface Plasmon Resonance (SPR) (BIACORE)

Binding of the generated antibodies to the respective antigens is investigated by surface plasmon resonance using a BIACORE instrument (GE Healthcare Biosciences AB, Uppsala, Sweden). Briefly, for affinity measurements Goat-Anti-Human IgG, JIR 109-005-098 antibodies are immobilized on a CM5 chip via amine coupling for presentation of the antibodies against the respective antigen. Binding is measured in HBS buffer (HBS-P (10 mM HEPES, 150 mM NaCl, 0.005% Tween 20, ph 7.4), 25° C. (or alternatively at 37° C.). Antigen (R&D Systems or in house purified) was added in various concentrations in solution. Association was measured by an antigen injection of 80 seconds to 3 minutes; dissociation was measured by washing the chip surface with HBS buffer for 3-10 minutes and a KD value was estimated using a 1:1 Langmuir binding model. Negative control data (e.g. buffer curves) are subtracted from sample curves for correction of system intrinsic baseline drift and for noise signal reduction. The respective Biacore Evaluation Software is used for analysis of sensorgrams and for calculation of affinity data.

### Example 1

### Generation and Production of Bispecific or Trispecific Antibodies Targeting CD28 and Fibroblast Activation Protein (FAP) or Carcinoembryonic Antigen (CEA)

1.1 Cloning of Bispecific or Trispecific Antibodies Targeting CD28 and Fibroblast Activation Protein (FAP) or Carcinoembryonic Antigen (CEA)

Cloning of the Antigen:

A DNA fragment encoding the extracellular domain (amino acids 1 to 134 of matured protein) of human CD28 (Uniprot: P10747) was inserted in frame into two different mammalian recipient vectors upstream of a fragment encoding a hum IgG1 Fc fragment which serves as solubility- and purification tag. One of the expression vectors contained the “hole” mutations in the Fc region, the other one the “knob” mutations as well as a C-terminal avi tag (GLNDIFEAQKIEWHE, SEQ ID NO:162) allowing specific biotinylation during co-expression with Bir A biotin ligase. In addition, both Fc fragments contained the PG-LALA mutations. Both vectors were co-transfected in combination with a plasmid coding for the BirA biotin ligase in order to get a dimeric CD28-Fc construct with a monovalent biotinylated avi-tag at the C-terminal end of the Fc-knob chain.

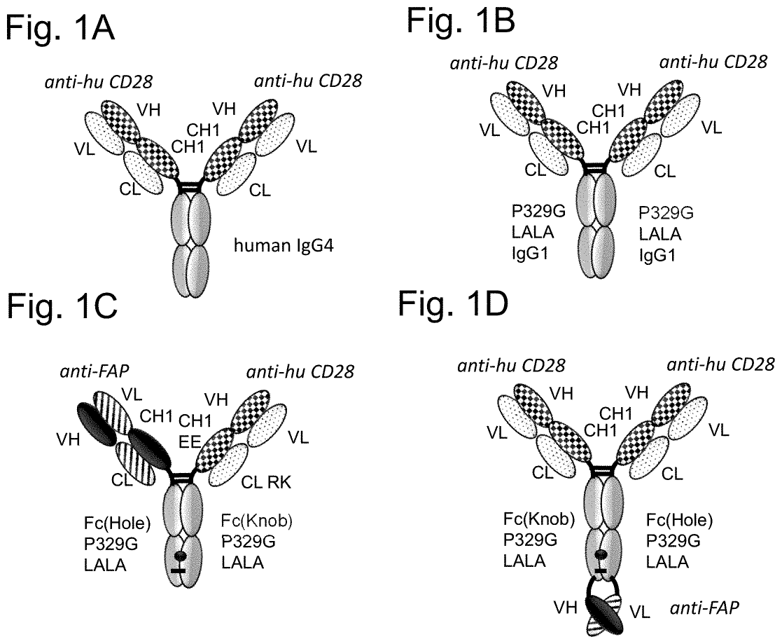

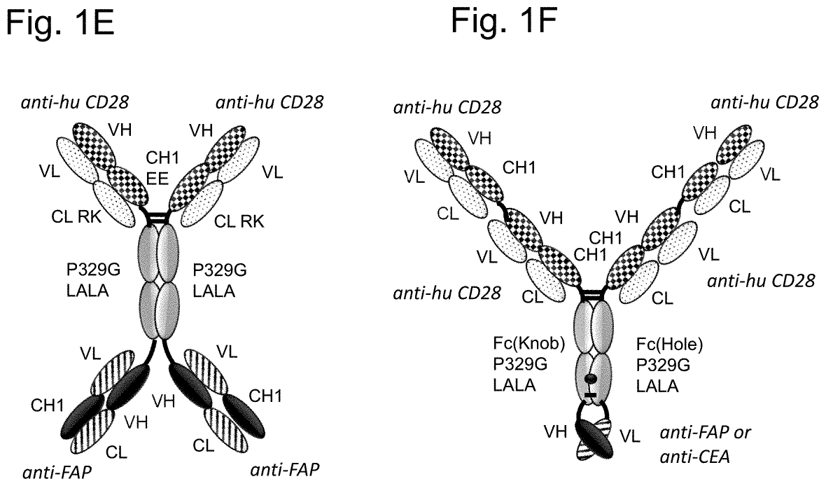

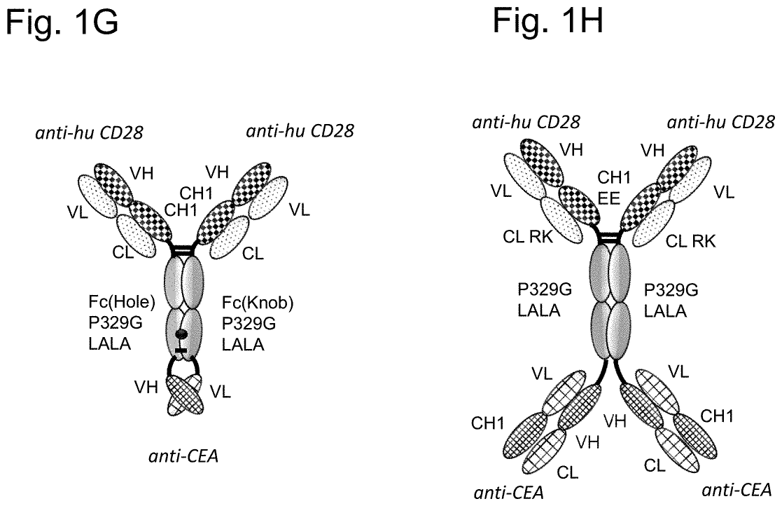

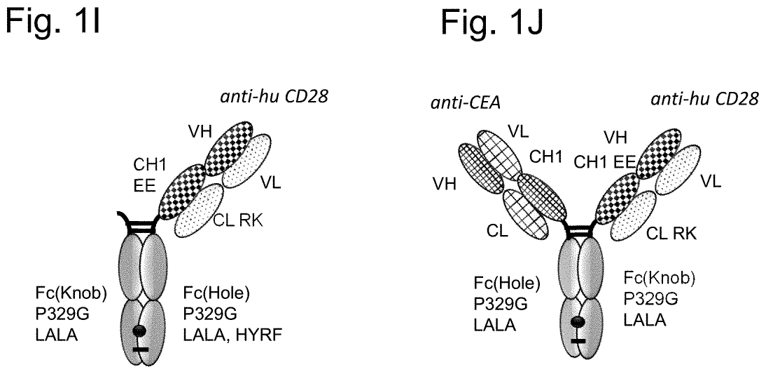

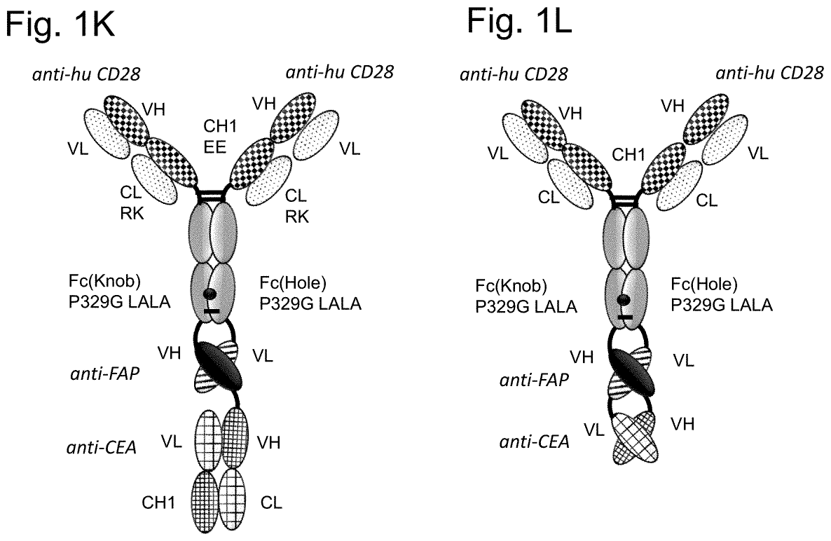

The variable domains of the FAP clone 4B9, a CEA binder and the CD28 clones SA and mAb 9.3 were used for the generation of various tumor targeted CD28 constructs. The generation and preparation of FAP clone 4B9 is disclosed in WO 2012/020006 A2, which is incorporated herein by reference. The CEA clone called MEDI-565 herein is described in WO 2007/071422 and the CD28 superagonistic antibody (SA) is described in WO 2006/050949. A description of antibody mAb 9.3 can be found in Tan et al. J. Immunology 2002, 169, 1119-1125. For the generation of the respective expression plasmids, the sequences of the respective variable domains were used and sub-cloned in frame with the respective constant regions which are pre-inserted in the respective recipient mammalian expression vector. A schematic description of the resulting molecules is shown in FIGS. 1A to 1L. Where indicated, Pro329Gly, Leu234Ala and Leu235Ala mutations (PG-LALA) have been introduced in the constant region of the human IgG1 heavy chains to abrogate binding to Fc gamma receptors. For the generation of unsymmetric bispecific antibodies, Fc-fragments contained either the “knob” or “hole” mutations to avoid mispairng of the heavy chains. In order to avoid mispairing of light chains in bi- and multispecific antibody constructs, exchange of VH/VL or CH1/Ckappa domains was introduced in one binding moiety (CrossFab technology). In another binding moiety, charges were introduced into the CH1 and Ckappa domains.

The following molecules were cloned, a schematic illustration of specific molecules is shown in FIGS. 1A to 1L:

Molecule A: CD28(SA) (hu IgG4), TGN1412, CD28 (SA) antibody in a human IgG4 isotype (FIG. 1A), comprises the amino acid sequences of SEQ ID NO:62 and SEQ ID NO:63 (P1AE1975).

Molecule B: CD28(SA) (PG-LALA), CD28 (SA) antibody in a huIgG1 PG-LALA isotype (FIG. 1B) comprises the amino acid sequences of SEQ ID NO:62 and SEQ ID NO:64 (P1AD9289).

Molecule C: FAP(4B9)-CD28(SA) 1+1 format, bispecific huIgG1 PG-LALA CrossFab molecule with charged modifications in the CD28(SA) Fab fragment (knob) and VH/VL exchange in FAP(4B9) Fab fragment (hole) (FIG. 1C) comprising the amino acid sequences of SEQ ID NOs: 65, 66, 67 and 68 (P1AD4492).

Molecule D: FAP(4B9)-CD28(SA) 1+4 format, bispecific tetravalent anti-CD28 (SA) and monovalent anti-FAP huIgG1 PG-LALA construct. The VH and VL domains of the FAP clone 4B9 were fused to the C-terminal end of respective chains of the Fc domain (VH: knob chain, VL: hole chain) (FIG. 1F). The molecule comprises the amino acid sequences of SEQ ID NOs: 62, 69 and 70 (P1AD9018).

Molecule E: FAP(4B9)-CD28(SA) 1+2 format, bispecific bivalent anti-CD28 (SA) and monovalent anti-FAP huIgG1 PG-LALA construct. The VH and VL domains of the FAP clone 4B9 were fused to the C-terminal end of respective chains of the Fc domain (VH: knob chain, VL: hole chain) (FIG. 1D). The molecule comprises the amino acid sequences of SEQ ID NOs: 62, 71 and 72 (P1AD9011).

Molecule F: FAP(4B9)-CD28(SA) 2+2, bispecific bivalent anti-CD28 (SA) and bivalent anti-FAP huIgG1 PG-LALA CrossFab construct, charged modifications in the anti-CD28 Fab fragments, VH fusion of the anti-FAP CrossFab fragments with CH1/Ckappa exchange to the C-terminal end of the Fc fragment (FIG. 1E). The molecule comprises the amino acid sequences of SEQ ID NOs:65, 73 and 74 (P1AD4493).

Molecule G: FAP (4B9)-CD28 (SA) 2+1, bispecific monovalent anti-CD28 (SA) and bivalent anti-FAP huIgG1 PG-LALA CrossFab construct, “classical orientation”, VH/VL exchange in the anti-CD28 CrossFab fragment, charged modification in anti-FAP Fab fragments. The molecule comprises the amino acid sequences of SEQ ID NOs: 75, 76, 77 and 78 (P1AD5231).

Molecule H: FAP(4B9)-CD28(SA)C-01, 1+1 bispecific monovalent anti-CD28 (SA) and monovalent anti-FAP huIgG1 PG-LALA CrossFab molecule, “head-to-tail”, VH/VL exchange in anti-CD28 CrossFab fragment, charged modification in anti-FAP binder. The molecule comprises the amino acid sequences of SEQ ID NOs: 75, 77, 78 and 79 (P1AE2021).

Molecule I: FAP(4B9)-CD28(SA)C-04, 1+1 bispecific monovalent anti-CD28 (SA) and monovalent anti-FAP huIgG1 PG-LALA construct. The VH and VL domains of the FAP binder 4B9 were fused to the C-terminal end of respective chains of the Fc fragment (VH: knob chain, VL: hole chain). The molecule comprises the amino acid sequences of SEQ ID NOs: SEQ ID NO: 62, 72 and 80 (P1AE2236).

Molecule J: CEA(Medi565)-CD28SA) 2+2, bispecific bivalent anti-CD28 (SA) and bivalent anti-CEA huIgG1 PG-LALA CrossFab construct, charged modifications in the anti-CD28 Fab fragments, VH fusion of the anti-CEA CrossFab fragment with CH1/Ckappa exchange to the C-terminal end of the Fc fragment (FIG. 111). The molecule comprises the amino acid sequences of SEQ ID NOs: 65, 81 and 82 (P1AE1195).

Molecule K: CEA(Medi565)-CD28(SA) 1+2, bispecific bivalent anti-CD28 (SA) and monovalent anti-CEA huIgG1 PG-LALA construct. The VH and VL domains of the CEA binder were fused to the C-terminal end of respective chains of the Fc fragment (VH: knob chain, VL: hole chain) (FIG. 1G). The molecule comprises the amino acid sequences of SEQ ID NOs: 62, 83 and 84 (P1AE1194).

Molecule L: monovalent IgG CD28 (SA), monovalent anti-CD28 (SA) huIgG1 PG-LALA construct, wherein the CD28 heavy chain is expressed as a “hole” Fc chain in combination with a Fc (knob) fragment (FIG. 1I). The molecule comprises the amino acid sequences of SEQ ID NOs: 65, 85 and 86 (P1AD8944).

Molecule M: CEA-CD28(SA) 1+1 format, bispecific huIgG1 PG-LALA CrossFab molecule with charged modifications in the CD28(SA) Fab fragment (knob) and VH/VL exchange in CEA crossFab fragment (hole) (FIG. 1J) comprising the amino acid sequences of SEQ ID NOs: 65, 66, 87 and 88 (P1AE3127).

Molecule N: mab 9.3 (PG-LALA), mAb9.3 clone in human IgG1 PG-LALA isotype (as in FIG. 1B). The molecule comprises the amino acid sequences of SEQ ID NOs: 89 and 90 (P1AD5142).

Molecule O: FAP(4B9)-CD28(mAb9.3)C-03, bispecific huIgG1 PG-LALA CrossFab construct with charged modifications in the mAb9.3 Fab fragment (knob) and VH/VL exchange in the anti-FAP fragment (hole) (as in FIG. 1C). The molecule comprises the amino acid sequences of SEQ ID NOs: 67, 68, 91 and 92 (P1AE2238).

Molecule P: FAP(4B9)-CD28(mAb9.3) 1+4, bispecific tetravalent anti-CD28 mAb9.3 and anti-FAP huIgG1 PG-LALA construct. The VH and VL domains of the FAP binder are fused to the C-terminal end of respective chains of the Fc fragment (VH: knob chain, VL: hole chain) (as in FIG. 1F). The molecule comprises the amino acid sequences of SEQ ID NOs: 89, 93 and 94 (P1AD8969).

Molecule Q: FAP(4B9)-CD28(mAb9.3) 1+2, bispecific bivalent anti-CD28 mAb9.3 and monovalent anti-FAP huIgG1 PG-LALA construct. The VH and VL domains of the FAP binder were fused to the C-terminal end of respective chains of the Fc fragment (VH: knob chain, VL: hole chain) (as in FIG. 1D). The molecule comprises the amino acid sequences of SEQ ID Nos: 89, 95 and 96 (P1AD8962).

Molecule R: FAP(4B9)-CD28(mAb9.3) 2+2, bispecific bivalent anti-CD28 mAb9.3 and bivalent anti-FAP huIgG1 PG-LALA CrossFab construct, charged modifications in the mAb9.3 FAP fragment, VH fusion of the anti-FAP Fab fragment with CH1/Ckappa CrossFab exchange to the C-terminal end of the Fc fragment (as in FIG. 1E). The molecule comprises the amino acid sequences of SEQ ID Nos: 97, 98 and 99 (P1AD8968).

Molecule S: FAP (4B9)-CD28(mAb9.3) 2+1, bispecific monovalent anti-CD28 (mAb9.3) and bivalent anti-FAP huIgG1 PG-LALA CrossFab construct, “classical orientation”, VH/VL exchange in the anti-CD28 (mAb9.3) CrossFab fragment, charged modification in anti-FAP Fab fragments. The molecule comprises the amino acid sequences of SEQ ID Nos: 76, 77, 100 and 101 (P1AD5560).

Molecule T: FAP(4B9)-CD28(mAb9.3)C-02, bispecific monovalent anti-CD28 (mAb9.3) and monovalent anti-FAP huIgG1 PG-LALA CrossFab construct, “head-to-tail”, VH/VL exchange in the anti-CD28 (mAb9.3) CrossFab fragment, charged modification in the anti-FAP fragment. The molecule comprises the amino acid sequences of SEQ ID Nos: 78, 79, 100 and 101 (P1AE2022).

Molecule U: FAP(4B9)-CD28(mAb9.3)C-05, bispecific monovalent anti-CD28 (mAb9.3) and monovalent anti-FAP huIgG1 PG-LALA construct. The VH and VL domains of the FAP binder 4B9 were fused to the C-terminal end of respective chains of the Fc fragment (VH: Fc knob chain, VL: Fc hole chain). The molecule comprises the amino acid sequences of SEQ ID Nos: 80, 89 and 96 (P1AE2237).

Molecule V: CEA-CD28(mAb9.3) 2+2, bispecific bivalent anti-CD28 (mAb9.3) and bivalent anti-CEA huIgG1 PG-LALA CrossFab construct, charged modifications in the mAb9.3 Fab fragment, VH fusion of the anti-CEA CrossFab fragment with CH1/Ckappa exchange to the C-terminal end of the Fc fragment (as in FIG. 111). The molecule comprises the amino acid sequences of SEQ ID Nos: 82, 89 and 102 (P1AE1193).

Molecule W: CEA-CD28(mAb9.3) 1+2, bispecific bivalent anti-CD28 (mAb9.3) and monovalent anti-CEA huIgG1 PG-LALA construct. The VH and VL domains of the CEA binder were fused to the C-terminal end of respective chains of the Fc fragment (VH: knob chain, VL: hole chain) (as in FIG. 1G). The molecule comprises the amino acid sequences of SEQ ID Nos: 89, 103 and 104 (P1AE1192).

Molecule X: monovalent IgG CD28 (mAb9.3), wherein the CD28 heavy chain is expressed as a “hole” Fc chain in combination with a Fc (knob) fragment (as in FIG. 1I). The molecule comprises the amino acid sequences of SEQ ID Nos: 86, 105 and 106 (P1AD8938).

Molecule Y: FAP(4B9)-CEA-CD28(SA) 1+1+2, trispecific bivalent anti-CD28, monovalent anti-FAP and monovalent anti-CEA huIgG1 PG-LALA construct. The VH and VL domains of the FAP binder were fused to the C-terminal end of respective chains of the Fc fragment (VH domain of FAP: knob chain, VL domain of FAP: hole chain). VH fusion of the anti-CEA Fab fragment to the C-terminal end of the FAP VH with CH1/Ckappa CrossFab exchange (FIG. 1K). The molecule comprises the amino acid sequences of SEQ ID Nos: 65, 107, 108 and 109 (P1AE0487).

Molecule Z: FAP(4B9)-CEA-CD28(SA) 1+1+2, trispecific bivalent anti-CD28, monovalent anti-FAP and monovalent anti-CEA huIgG1 PG-LALA construct. The VH and VL domains of the FAP and CEA binders were fused to the C-terminal end of respective chains of the Fc fragment (VH domains of FAP and CEA: knob chain, VL domains of FAP and CEA: hole chain) (FIG. 1L). The molecule comprises the amino acid sequences of SEQ ID Nos: 62, 110 and 111 (P1AE0486).

1.2 Production of Bispecific or Trispecific Antibodies Targeting CD28 and Fibroblast Activation Protein (FAP) or Carcinoembryonic Antigen (CEA)

Expression of the above-mentioned molecules is either driven by a chimeric MPSV promoter or a CMV promoter. Polyadenylation is driven by a synthetic polyA signal sequence located at the 3′ end of the CDS. In addition, each vector contains an EBV OriP sequence for autosomal replication.

For the production of the constructs C to W, HEK293-EBNA cells that grow in suspension were co-transfected with the respective expression vectors using polyethylenimine as a transfection reagent. Antibodies and bispecific antibodies were generated by transient transfection of HEK293 EBNA cells. Cells were centrifuged and medium replaced by pre-warmed CD CHO medium. Expression vectors were mixed in CD CHO medium, PEI was added, the solution vortexed and incubated for 10 minutes at room temperature. Afterwards, cells were mixed with the DNA/PEI solution, transferred to shake flask and incubated for 3 hours at 37° C. in an incubator with a 5% CO2 atmosphere. After the incubation, Excell medium with supplements was added (Mammalian Cell Cultures for Biologics Manufacturing, Editors: Weichang Zhou, Anne Kantardjieff). One day after transfection supplements (Feed) were added (Mammalian Cell Cultures for Biologics Manufacturing, Editors: Weichang Zhou, Anne Kantardjieff). Cell supernatants were harvested after 7 days by centrifugation and subsequent filtration (0.2 μm filter) and purified by standard methods.

Constructs A, B, X and Y were prepared by Evitria using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). Supernatant was harvested by centrifugation and subsequent filtration (0.2 μm filter) and purified by standard methods.

1.3 Purification of Bispecific or Trispecific Antibodies Targeting CD28 and Fibroblast Activation Protein (FAP) or Carcinoembryonic Antigen (CEA)

Proteins were purified from filtered cell culture supernatants referring to standard protocols. In brief, Fc-containing proteins were purified from cell culture supernatants by affinity chromatography using Protein A. Elution was achieved at pH 3.0 followed by immediate neutralization of the sample. The protein was concentrated and aggregated protein was separated from monomeric protein by size exclusion chromatography in 20 mM histidine, 140 mM sodium chloride, pH 6.0.

1.4 Analytical Data of Bispecific or Trispecific Antibodies Targeting CD28 and Fibroblast Activation Protein (FAP) or Carcinoembryonic Antigen (CEA)

The protein concentration of purified constructs was determined by measuring the optical density (OD) at 280 nm, using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII (Perkin Elmer). Determination of the aggregate content was performed by HPLC chromatography at 25° C. using analytical size-exclusion column (TSKgel G3000 SW XL or UP-SW3000) equilibrated in running buffer (25 mM K₂HPO₄, 125 mM NaCl, 200 mM L-Arginine Monohydrocloride, pH 6.7 or 200 mM KH₂PO₄, 250 mM KCl pH 6.2 respectively). A summary of the purification parameters of all molecules is given in Table 1.

TABLE 1
Summary of the production and purification of bispecific or trispecific CD28
antigen binding molecules

| | | | Analytical SEC | |
|---|---|---|---|---|
| | | | (HMW/Monomer/L | |
| | | Yield | MW) | Purity measured |
| Molecule | Description | [mg/l] | [%] | by CE-SDS [%] |
| A | CD28(SA) | 257 | 0/100/0 | 84.25 |
| | (hu IgG4) | | | |
| B | CD28(SA) | 390 | 0/97.3/2.7 | 84 |
| | hu IgG1 (PG-LALA) | | | |
| C | FAP(4B9)-CD28(SA) | 19.5 | 0.64/97.28/2.07 | 98.75 |
| | 1 + 1 | | | |
| D | FAP(4B9)- | 1.75 | 3.53/96.48/0 | n.d. |
| | CD28(TGN1412) 1 + 4 | | | |
| E | FAP(4B9)-CD28(SA) | 0.38 | 0.8/95.48/3.72 | 93.58 |
| | 1 + 2 | | | |
| F | FAP(4B9)-CD28(SA) | 18.2 | 1.4/98.6/0 | 91.42 |
| | 2 + 2 | | | |
| G | FAP(4B9)-CD28(SA) | 2.66 | 3.79/94.02/2.19 | 64 |
| | 2 + 1 | | | |
| H | FAP(4B9)-CD28(SA) | 10.6 | 0/100/0 | 99.38 |
| | C-01 | | | |
| I | FAP(4B9)-CD28(SA) | 5.55 | 4.12/ 81.17/14.71 | 96.5 |
| | C-04 | | | |
| J | CEA-CD28(SA) 2 + 2 | 6.25 | 1/99/0 | n.d. |
| K | CEA-CD28(SA) 1 + 2 | 5.8 | 0.5/99.5/0 | 64 |
| L | monovalent IgG1 CD28 | 38.5 | 0.2/99.6/0.2 | 99.3 |
| | (SA) | | | |
| M | CEA-CD28(SA) 1 + 1 | 14.3 | 0/100/0 | 99.18 |
| N | CD28(mAb 9.3) | 22.06 | 0/100/0 | 88 |
| | hu IgG1 (PG-LALA) | | | |
| O | FAP(4B9)- | 2.14 | 0/100/0 | 97.4 |
| | CD28(mAb9.3) C-03 | | | |
| P | FAP(4B9)- | 7.6 | 1.2/98.8/0 | 97.6 |
| | CD28(mAb9.3) 1 + 4 | | | |
| Q | FAP(4B9)- | 16. | 1/98.5/0.5 | 97.16 |
| | CD28(mAb9.3) 1 + 2 | | | |
| R | FAP(4B9)- | 3.9 | 0/95.5/4.5 | 87 |
| | CD28(mAb9.3) 2 + 2 | | | |
| S | FAP(4B9)-CD28 | 2.63 | 2.1/96.3/1.6 | 90.55 |
| | (mAb9.3) 2 + 1 | | | |
| T | FAP(4B9)- | 2.3 | 0/100/0 | 100 |
| | CD28(mAb9.3) C-02 | | | |
| U | FAP(4B9)- | 23.78 | 0.68/97.82/1.5 | 96.1 |
| | CD28(mAb9.3) C-05 | | | |
| V | CEA-CD28(mAb9.3) | 3.1 | 0/100/0 | 100 |
| | 2 + 2 | | | |
| W | CEA-CD28(mAb9.3) | 2.25 | 0/100/0 | 92.8 |
| | 1 + 2 | | | |
| X | monovalent IgG1 | 20.2 | 1.4/98.6/0 | 97.7 |
| | CD28 (mAb9.3) | | | |
| Y | FAP(4B9)-CEA- | 11.3 | 10.7/85/4.3 | 85 |
| | CD28(SA) 1 + 1 + 2 | | | |
| Z | FAP(4B9)-CEA- | 11.8 | 4.6/95.4/0 | 73.6 |
| | CD28(SA) 1 + 1 + 2 | | | |

### Example 2

### Binding and Kinetic Analysis of Bispecific Antibodies of Bispecific or Trispecific Antibodies Targeting CD28 and Fibroblast Activation Protein (FAP) or Carcinoembryonic Antigen (CEA)

2.1 Binding of Bispecific Antibodies Targeting CD28 and Fibroblast Activation Protein (FAP) to FAP- and CD28-Expressing Cells

The binding of bispecific FAP-CD28 molecules was tested using human fibroblast activating protein (huFAP) expressing 3T3-huFAP cells (clone 19). This cell line was generated by the transfection of the mouse embryonic fibroblast NIH/3T3 cell line (ATCC CRL-1658) with the expression vector pETR4921 to express huFAP under 1.5 μg/mL Puromycin selection. The binding to human CD28 was tested with CHO cells expressing human CD28 (parental cell line CHO-k1 ATCC # CCL-61, modified to stably overexpress human CD28).

To assess binding, cells were harvested, counted, checked for viability and re-suspended at 2.5E5/ml in FACS buffer (eBioscience, Cat No 00-4222-26). 5×10⁴ cells were incubated in round-bottom 96-well plates for 2 h at 4° C. with increasing concentrations of the FAP-targeted CD28 constructs (1 pM-100 nM). Then, cells were washed three times with cold FACS buffer, incubated for further 60 min at 4° C. with PE-conjugated, goat-anti human PE (Jackson ImmunoReserach, Cat No 109-116-098), washed once with cold FACS buffer, centrifuged and resuspended in 100 μl FACS buffer. To monitor unspecific binding interactions between constructs and cells, an anti-DP47 IgG was included as negative control. Binding was assessed by flow cytometry with a FACS Fortessa (BD, Software FACS Diva). Binding curves were obtained using GraphPadPrism6.

The FAP-CD28 molecules were able to bind to both human FAP as well as human CD28 on cells in a concentration dependent manner (FIGS. 2B and 2C for certain examples). As expected, no binding was detected with the anti-DP47 IgG, indicating that the detection of binding is due to specific CD28 and FAP binding by the respective targeting moieties.

2.2 Kinetic Analysis of Bispecific or Trispecific Antibodies Targeting CD28 and CEA

Affinity (K_{D}) of both binding moieties of the bispecific or trispecific antibodies comprising anti-CEA (Medi-565) and anti-CD28 was measured by SPR using a ProteOn XPR36 instrument (Biorad) at 25° C. with biotinylated huCD28-Fc antigen and biotinylated Hu N(A2-B2)A-avi-His immobilized on an NLC chip by neutravidin capture.

For the generation of a CEACAM5-based antigen that contains the epitope for CEA(Medi-565), a chimeric protein consisting of two CEACAM1 and two CEACAM5 Ig domains was generated. Based on the sequence of CEACAM1, the second and third domain of CEACAM1 was replaced by the CEACAM5 domains A2 and B2. A C-terminal avi-tag and His tag were fused for site-specific biotinylation and purification. The resulting protein was named Hu N(A2-B2)A-avi-His (SEQ ID NO: 161).

Immobilization of recombinant antigens (ligand): Antigens were diluted with PBST (10 mM phosphate, 150 mM sodium chloride pH 7.4, 0.005% Tween 20) to 10 μg/ml, then injected at 30 μl/minute at varying contact times, to achieve immobilization levels of about 400, 800, and 1600 response units (RU) in vertical orientation. Injection of analytes: For one-shot kinetics measurements, injection direction was changed to horizontal orientation, two-fold dilution series of the purified bispecific CEA-targeted anti-CD28 bispecific antibody (varying concentration ranges between 50 and 3.125 nM) were injected simultaneously at 50 μl/min along separate channels 1-5, with association times of 150 s, and dissociation times of 450 s. Buffer (PBST) was injected along the sixth channel to provide an “in-line” blank for referencing. Association rate constants (kon) and dissociation rate constants (koff) were calculated using a simple one-to-one Langmuir binding model in ProteOn Manager v3.1 software by simultaneously fitting the association and dissociation sensorgrams. The equilibrium dissociation constant (K_{D}) was calculated as the ratio koff/kon. Calculated K_{D} values of a bispecific antibody comprising one anti-CD28 antigen binding domain and one anti-CEA antigen binding domain (Molecule M) are in line with the measured values of the respective monospecific constructs. The kinetic and thermodynamic data are summarized in Table 2 below.

TABLE 2
kinetic and thermodynamic analysis of CEA-CD28(SA) 1 + 1
(Molecule M)

| Binding moiety | kₒₙ (1/(s * M) | k_{off} (1/s) | K_{D} (nM) |
|---|---|---|---|
| Anti-CEA (Medi-565) | 4.13 exp5 | 1.2 exp−4 | 0.29 |
| Anti-CD28 (TGN1412) | 3.13 exp5 | 3.76 exp−4 | 1.2 |

### Example 3

### Generation and Characterization of CD28 (SA) Variants Devoid of Hotspots and Reduced in Affinity

3.1 Removal of an Unpaired Cysteine Residue, Tryptophan Residues, a Deamidation Site and Generation of Affinity-Reduced CD28 (SA) Variants

As part of our detailed binder characterization, a computational analysis of the CD28(SA) variable domain sequences was performed. This analysis revealed an unpaired cysteine in the CDR2 region of VH (position 50, Kabat numbering), tryptophan residues in CDR3 of VH (position 100a, Kabat numbering) and CDR1 of VL (position 32, Kabat numbering), and a potential asparagine deamidation site in CDR2 of VH (position 56, Kabat numbering). While oxidation of tryptophan is a rather slow process and can be prevented by adding reducing compounds, the presence of unpaired cysteines in an antibody variable domain can be critical. Free cysteines are reactive and can form stable bonds with other unpaired cysteines of other proteins or components of the cell or media. As a consequence, this can lead to a heterogeneous and instable product with unknown modifications which are potentially immunogenic and therefore may pose a risk for the patients. In addition, deamidation of asparagine and the resulting formation of iso-aspartate and succinimide can affect both in vitro stability and in vivo biological functions. A crystal structure analysis of the parental murine binder 5.11A revealed that C50 is not involved in binding to human CD28 and therefore can be replaced by a similar amino acid such as serine without affecting the affinity to CD28 (Table 5, variant 29). Both tryptophan residues as well as asparagine at position 50, however, are close to or involved in the binding interface and a replacement by a similar amino acid can therefore lead to a reduction of the binding affinity. In this example, we particularly aimed at reducing the affinity of CD28(SA) to human CD28 because of the following reason: The affinity of CD28(SA) is in the range of 1-2 nM with a binding half-life of about 32 minutes. This strong affinity can lead to a sink effect in tissue containing large amounts of CD28-expressing cells such as blood and lymphatic tissue when injected intravenously into patients. As a consequence, site-specific targeting of the compound via the targeting component(s) FAP and/or CEA may be reduced and the efficacy of the construct can be diminished. In order to minimize such an effect, several VH and VL variants were generated in order to reduce to affinities to different degrees (FIGS. 3A and 3C). Besides the previously mentioned positions that represent potential stability hotspots, additional residues involved directly or indirectly in the binding to human CD28 were replaced either by the original murine germline amino acid or by a similar amino acid. In addition, the CDRs of both CD28(SA) VL and VH were also grafted into the respective framework sequences of trastuzumab (FIGS. 3B and 3D). Several combinations of VH and VL variants were then expressed as monovalent one-armed anti-CD28 IgG-like constructs and binding was characterized by SPR.

3.2 Analysis of the Dissociation Rate Constants (Koff) of Reduced One-Armed Anti-CD28 Variants by SPR

In order to characterize the anti-CD28 binder variants in a first step, all binders were expressed as monovalent one-armed IgG-like constructs (FIG. 4A). This format was chosen in order to characterize the binding to CD28 in a 1:1 model. 5 days after transfection into HEK cells, the supernatant was harvested and the titer of the expressed constructs was determined.

The off-rate of the anti-CD28 binder variants was determined by surface plasmon resonance (SPR) using a ProteOn XPR36 instrument (Biorad) at 25° C. with biotinylated huCD28-Fc antigen immobilized on NLC chips by neutravidin capture. For the immobilization of recombinant antigen (ligand), huCD28-Fc was diluted with PBST (Phophate buffered saline with Tween 20 consisting of 10 mM phosphate, 150 mM sodium chloride pH 7.4, 0.005% Tween 20) to concentrations ranging from 100 to 500 nM, then injected at 25 μl/minute at varying contact times. This resulted in immobilization levels between 1000 to 3000 response units (RU) in vertical orientation.

For one-shot kinetics measurements, injection direction was changed to horizontal orientation. Based on the titer of the produced supernatants, the monovalent one-armed IgGs were diluted with PBST to get two-fold dilution series ranging from 100 nM to 6.25 nM. Injection was performed simultaneously at 50 μl/min along separate channels 1-5, with association times of 120 s, and dissociation times of 300 s. Buffer (PBST) was injected along the sixth channel to provide an “in-line” blank for referencing. Since the binding interaction was measured with monovalent one-armed IgGs from supernatant without purification and biochemical characterization, only the off-rates of the protein:protein interaction was used for further conclusions. Off-rates were calculated using a simple one-to-one Langmuir binding model in ProteOn Manager v3.1 software by fitting the dissociation sensorgrams. The dissociation rate constants (k_{off}) values of all clones are summarized in Table 2. Comparison of the produced variants revealed k_{off} values with an up to 30-fold decrease compared to the parental sequence.

TABLE 2
Summary of all expressed monovalent anti-CD28 variants with
dissociation rate constants (k_{off}) values

| | | SEQ ID | SEQ ID | | k_{off} |
|---|---|---|---|---|---|
| Binder variants | Tapir ID | NO: | NO: | SEQ ID NO: | (10⁻⁴/M) |
| CD28(SA)_variant_1 | P1AE4441 | 112 | 65 | 126 | 3.0 |
| (parental CD28) | | | | | |
| CD28(SA)_variant_2 | P1AE3058 | 113 | 120 | 126 | N/A |
| CD28(SA)_variant_3 | P1AE3059 | 113 | 121 | 126 | N/A |
| CD28(SA)_variant_4 | P1AE3060 | 113 | 122 | 126 | N/A |
| CD28(SA)_variant_5 | P1AE3061 | 113 | 65 | 126 | N/A |
| CD28(SA)_variant_6 | P1AE3062 | 114 | 120 | 126 | N/A |
| CD28(SA)_variant_7 | P1AE3063 | 114 | 121 | 126 | 100 |
| CD28(SA)_variant_8 | P1AE3064 | 114 | 122 | 126 | 68 |
| CD28(SA)_variant_9 | P1AE3065 | 114 | 123 | 126 | 78 |
| CD28(SA)_variant_10 | P1AE3066 | 114 | 124 | 126 | N/A |
| CD28(SA)_variant_11 | P1AE3067 | 114 | 65 | 126 | 37 |
| CD28(SA)_variant_12 | P1AE3068 | 115 | 125 | 126 | 2.4 |
| CD28(SA)_variant_13 | P1AE3069 | 115 | 65 | 126 | 1.9 |
| CD28(SA)_variant_14 | P1AE3070 | 116 | 120 | 126 | 100 |
| CD28(SA)_variant_15 | P1AE3071 | 116 | 121 | 126 | 24 |
| CD28(SA)_variant_16 | P1AE3072 | 116 | 122 | 126 | 10 |
| CD28(SA)_variant_17 | P1AE3073 | 116 | 123 | 126 | 14 |
| CD28(SA)_variant_18 | P1AE3074 | 116 | 124 | 126 | 82 |
| CD28(SA)_variant_19 | P1AE3075 | 116 | 65 | 126 | 2.9 |
| CD28(SA)_variant_20 | P1AE3076 | 117 | 120 | 126 | N/A |
| CD28(SA)_variant_21 | P1AE3077 | 117 | 121 | 126 | N/A |
| CD28(SA)_variant_22 | P1AE3078 | 117 | 122 | 126 | 61 |
| CD28(SA)_variant_23 | P1AE3079 | 117 | 65 | 126 | 43 |
| CD28(SA)_variant_24 | P1AE3080 | 118 | 120 | 126 | 80 |
| CD28(SA)_variant_25 | P1AE3081 | 118 | 121 | 126 | 3.51 |
| CD28(SA)_variant_26 | P1AE3082 | 118 | 122 | 126 | 9.7 |
| CD28(SA)_variant_27 | P1AE3083 | 118 | 123 | 126 | 14 |
| CD28(SA)_variant_28 | P1AE3084 | 118 | 124 | 126 | 69 |
| CD28(SA)_variant_29 | P1AE3085 | 118 | 65 | 126 | 2.5 |
| CD28(SA)_variant_30 | P1AE3086 | 119 | 125 | 126 | 3.22 |
| CD28(SA)_variant_31 | P1AE3087 | 119 | 65 | 126 | 2.5 |

3.3 Preparation and Kinetic Analysis of Bispecific FAP-Targeted Anti-CD28 Affinity Variants

Based on the off-rate analysis and the binding study on CD28-expressing cells, several combinations of anti-CD28 VH and VL variants with different binding intensities were selected and expressed as FAP-targeted bispecific huIgG1 PG-LALA CrossFab molecules (for combinations of SEQ ID NO:s see Table 3). The resulting constructs in 1+1 format (FIG. 1C) were purified and a biochemical analysis was performed (Table 4).

TABLE 3
Summary of all expressed 1 + 1 bispecific FAP-targeted anti-CD28 variants

| | | SEQ ID | SEQ ID | SEQ ID | |
|---|---|---|---|---|---|
| Binder variants | Tapir ID | NO: | NO: | NO: | SEQ ID NO: |
| FAP (4B9)-CD28 | P1AE3131 | 67 | 68 | 114 | 122 |
| (CD28(SA)_Variant 8) 1 + 1 | | | | | |
| FAP (4B9)-CD28 | P1AE3132 | 67 | 68 | 114 | 65 |
| (CD28(SA)_Variant 11) 1 + 1 | | | | | |
| FAP (4B9)-CD28 | P1AE3133 | 67 | 68 | 115 | 125 |
| (CD28(SA)_Variant 12) 1 + 1 | | | | | |
| FAP (4B9)-CD28 | P1AE3134 | 67 | 68 | 116 | 121 |
| (CD28(SA)_Variant 15) 1 + 1 | | | | | |
| FAP (4B9)-CD28 | P1AE3135 | 67 | 68 | 116 | 122 |
| (CD28(SA)_Variant 16) 1 + 1 | | | | | |
| FAP (4B9)-CD28 | P1AE3136 | 67 | 68 | 116 | 123 |
| (CD28(SA)_Variant 17) 1 + 1 | | | | | |
| FAP (4B9)-CD28 | P1AE3137 | 67 | 68 | 116 | 65 |
| (CD28(SA)_Variant 19) 1 + 1 | | | | | |
| FAP (4B9)-CD28 | P1AE3138 | 67 | 68 | 117 | 65 |
| (CD28(SA)_Variant 23) 1 + 1 | | | | | |
| FAP (4B9)-CD28 | P1AE3139 | 67 | 68 | 118 | 121 |
| (CD28(SA)_Variant 25) 1 + 1 | | | | | |
| FAP (4B9)-CD28 | P1AE3140 | 67 | 68 | 118 | 123 |
| (CD28(SA)_Variant 27) 1 + 1 | | | | | |
| FAP (4B9)-CD28 | P1AE3141 | 67 | 68 | 118 | 65 |
| (CD28(SA)_Variant 29) 1 + 1 | | | | | |

TABLE 4
Summary of the production and purification of FAP-targeted anti- CD28 variants

| | | | Analytical SEC | Purity |
|---|---|---|---|---|
| | | Yield | (HMW/Monomer/ | measured by |
| TaPIR ID | Bispecific molecules | [mg/l] | LMW) [%] | CE-SDS [%] |
| P1AE3131 | FAP (4B9)-CD28 | 11.8 | 0.1/98.5/1.4 | 100 |
| | (CD28(SA)_Variant 8) 1 + 1 | | | |
| P1AE3132 | FAP (4B9)-CD28 | 8.1 | 0.5/97.4/2.1 | 100 |
| | (CD28(SA)_Variant 11) 1 + 1 | | | |
| P1AE3133 | FAP (4B9)-CD28 | 6.1 | 0/100/0′ | 100 |
| | (CD28(SA)_Variant 12) 1 + 1 | | | |
| P1AE3134 | FAP (4B9)-CD28 | 9.2 | 0/100/0 | 100 |
| | (CD28(SA)_Variant 15) 1 + 1 | | | |
| P1AE3135 | FAP (4B9)-CD28 | 0.4 | 0/100/0 | 97 |
| | (CD28(SA)_Variant 16) 1 + 1 | | | |
| P1AE3136 | FAP (4B9)-CD28 | 1.35 | 0/78.7/21.3 | 87 |
| | (CD28(SA)_Variant 17) 1 + 1 | | | |
| P1AE3137 | FAP (4B9)-CD28 | 2.6 | 0/100/0 | 100 |
| | (CD28(SA)_Variant 19) 1 + 1 | | | |
| P1AE3138 | FAP (4B9)-CD28 | 15.5 | 0/97.5/2.5 | 98 |
| | (CD28(SA)_Variant 23) 1 + 1 | | | |
| P1AE3139 | FAP (4B9)-CD28 | 5.4 | 0/88.7/11.3 | 100 |
| | (CD28(SA)_Variant 25) 1 + 1 | | | |
| P1AE3140 | FAP (4B9)-CD28 | 9.7 | 0/98.3/1.7 | 96 |
| | (CD28(SA)_Variant 27) 1 + 1 | | | |
| P1AE3141 | FAP (4B9)-CD28 | 1.76 | 1/99/0 | 96.3 |
| | (CD28(SA)_Variant 29) 1 + 1 | | | |

Affinity (K_{D}) of the produced bispecific antigen binding molecules to CD28 was measured by SPR using a ProteOn XPR36 instrument (Biorad) at 25° C. with biotinylated huCD28-Fc antigen immobilized on NLC chips by neutravidin capture. Immobilization of recombinant antigens (ligand): Antigen was diluted with PBST (10 mM phosphate, 150 mM sodium chloride pH 7.4, 0.005% Tween 20) to 10 μg/ml, then injected at 30 μl/minute at varying contact times, to achieve immobilization levels of about 200, 400 or 800 response units (RU) in vertical orientation. Injection of analytes: For one-shot kinetics measurements, injection direction was changed to horizontal orientation, two-fold dilution series of purified bispecific FAP-targeted anti-CD28 affinity variants (varying concentration ranges between 50 and 3.125 nM) were injected simultaneously at 50 μl/min along separate channels 1-5, with association times of 150 s, and dissociation times of 450 s. Buffer (PBST) was injected along the sixth channel to provide an “in-line” blank for referencing. Association rate constants (kₒₙ) and dissociation rate constants (k_{off}) were calculated using a simple one-to-one Langmuir binding model in ProteOn Manager v3.1 software by simultaneously fitting the association and dissociation sensorgrams. The equilibrium dissociation constant (K_{D}) was calculated as the ratio k_{off}/k_{off} Analyzed clones revealed K_{D} values in a broad range (between 1 and 25 nM). The kinetic and thermodynamic data are summarized in Table 5.

TABLE 5
kinetic and thermodynamic analysis of expressed FAP-targeted
anti-CD28 variants

| Bispecific molecule | kₒₙ (1/(s * M) | k_{off} (1/s) | K_{D} (nM) |
|---|---|---|---|
| parental | 3.79 exp5 | 3.6 exp−4 | 1 |
| FAP (4B9)-CD28 | 2.19 exp5 | 5.21 exp−3 | 23.8 |
| (CD28(SA)_Variant 8) 1 + 1 | | | |
| FAP (4B9) - CD28 | 2.3 exp5 | 2.87 exp−3 | 12.5 |
| (CD28(SA)_Variant 11) 1 + 1 | | | |
| FAP (4B9) - CD28 | 2.61 exp5 | 2.67 exp−4 | 1 |
| (CD28(SA)_Variant 12) 1 + 1 | | | |
| FAP (4B9) - CD28 | 2.59 exp5 | 1.84 exp−3 | 7.1 |
| (CD28(SA)_Variant 15) 1 + 1 | | | |
| FAP (4B9) - CD28 | 1.87 exp5 | 9.94 exp−4 | 5.3 |
| (CD28(SA)_Variant 16) 1 + 1 | | | |
| FAP (4B9) - CD28 | 3.38 exp5 | 1.25 exp−3 | 3.7 |
| (CD28(SA)_Variant 17) 1 + 1 | | | |
| FAP (4B9) - CD28 | 2.8 exp5 | 3.04 exp−4 | 1.1 |
| (CD28(SA)_Variant 19) 1 + 1 | | | |
| FAP (4B9) - CD28 | 2.11 exp5 | 3.42 exp−3 | 16.3 |
| (CD28(SA)_Variant 23) 1 + 1 | | | |
| FAP (4B9) - CD28 | 2.38 exp5 | 3.96 exp−4 | 1.7 |
| (CD28(SA)_Variant 25) 1 + 1 | | | |
| FAP (4B9) - CD28 | 2.27 exp5 | 1.21 exp−3 | 5.4 |
| (CD28(SA)_Variant 27) 1 + 1 | | | |
| FAP (4B9) - CD28 | 2.72 exp5 | 3.07 exp−4 | 1.1 |
| (CD28(SA)_Variant 29) 1 + 1 | | | |

### Example 4

### Binding of Monovalent CD28 Agonistic IgGs and FAP-Targeted CD28 Agonistic Antibodies to CD28-Expressing Cells

Binding to human CD28 was tested with CHO cells expressing human CD28 (parental cell line CHO-k1 ATCC # CCL-61, modified to stably overexpress human CD28). To assess binding, cells were harvested, counted, checked for viability and re-suspended at 2.5×10⁵/ml in FACS buffer (eBioscience, Cat No 00-4222-26). 5×10⁴ cells were incubated in round-bottom 96-well plates for 2 h at 4° C. with increasing concentrations of the CD28 binders (1 pM-100 nM). Then, cells were washed three times with cold FACS buffer, incubated for further 60 min at 4° C. with PE-conjugated, goat-anti human PE (Jackson ImmunoReserach, Cat No 109-116-098), washed once with cold FACS buffer, centrifuged and resuspended in 100 ul FACS buffer. To monitor unspecific binding interactions between constructs and cells, an anti-DP47 IgG was included as negative control. Binding was assessed by flow cytometry with a FACS Fortessa (BD, Software FACS Diva). Binding curves were obtained using GraphPadPrism6.

The monovalent one-armed IgG-like CD28 variant constructs showed differences in binding as can be seen from FIGS. 4A to 4C. Furthermore, the binding of bispecific FAP-targeted anti-CD28 antibodies in 1+1 format to CHO cells expressing human CD28 was determined. The K_{D} values for the different 1+1 constructs with selected CD28 variants are shown in Table 6 below or in the corresponding graphs of FIGS. 4D and 4E.

TABLE 6
Binding of FAP-targeted anti-CD28 1 + 1 constructs to CHO
cells expressing human CD28

| Binder | TAPIR | K_{D} (nM) |
|---|---|---|
| TGN1412 | P1AD4492 | 1 |
| variant 8 | P1AE3131 | 23.8 |
| variant 11 | P1AE3132 | 12.5 |
| variant 12 | P1AE3133 | 1 |
| variant 15 | P1AE3134 | 7.1 |
| variant 16 | P1AE3135 | 5.3 |
| variant 17 | P1AE3136 | 3.7 |
| variant 19 | P1AE3137 | 1.1 |
| variant 23 | P1AE3138 | 16.3 |
| variant 25 | P1AE3139 | 1.7 |
| variant 27 | P1AE3140 | 5.4 |
| Variant 29 | P1AE3141 | 1.1 |

### Example 5

### In Vitro Functional Characterization of Bispecific Antibodies Targeting CD28 and Fibroblast Activation Protein (FAP) to FAP- and CD28-Expressing Cells

Several cell-based in vitro assays were performed with primary human PBMCs to evaluate the activity of CD28(SA) and bispecific FAP-targeted CD28 antigen binding molecules in the presence and absence of TCR signals provided by T-cell bispecific-(TCB) antibodies. T-cell proliferation, cytokine secretion, and tumor cell killing as determined by flow cytometry, cytokine ELISA, and live cell imaging were obtained as read-outs.

1. The activity of the original superagonistic CD28(SA) IgG4 was assessed using a previously described high density pre-culture system to restore the responsiveness of peripheral blood derived T cells towards CD28-mediated superagonism (Römer et al., 2011).

2. The functionality of bispecific FAP-targeted CD28 molecules in the absence of TCR signals was assessed in a primary human PBMC co-culture assay, wherein bispecific FAP-targeted CD28 molecules were crosslinked by simultaneous binding to human CD28 on T cells and human FAP, expressed on either 3T3-huFAP cells (parental cell line ATCC # CCL-92, modified to stably overexpress human FAP) or MCSP- and FAP-expressing MV3 melanoma cells.

3. The functionality of bispecific FAP-targeted CD28 molecules in the presence of TCR signals was assessed as described above, with the additional presence of a TCB molecule, crosslinked by simultaneous binding to CD3 on T cells and, either human CEA on MKN45 gastric cancer cells (DSMZ # ACC 409), or MCSP, expressed on MV3 melanoma cells.

PBMC Isolation

Peripheral blood mononuclear cells (PBMCs) were prepared by density gradient centrifugation from enriched lymphocyte preparations of heparinized blood obtained from a Buffy Coat (Blutspende Zurich). 25 ml of blood (diluted 1:2 in PBS) were layered over 15 ml lymphoprep (STEMCELL technologies, Cat No 07851) and centrifuged at room temperature for 25 min at 845×g without brake. The PBMC-containing interphase was collected in 50 ml tubes with a 10 ml pipette. The cells were washed with PBS and centrifuged 5 min at 611×g. The supernatant was discarded, the pellet re-suspended in 50 ml PBS and centrifuged for 5 min at 304×g. The washing step was repeated, centrifuging at 171×g. The cells were re-suspended in RPMI 1640 Glutamax (containing 5% human serum, sodium pyruvate, NEAA, 50 μM 2-mercaptoethanol, Penicillin/Streptomycin) and processed for further functional analysis according to the respective assay protocol.

High Density Pre-Culture of PBMCs and In Vitro Assessment of T Cell Activation by the CD28 Superagonist CD28(SA)

To restore the responsiveness of human T cells to TGN1412-mediated CD28 superagonism, PBMCs were pre-cultured at high density (HD) (Römer et al, 2011) before assessing the effects of CD28 superagonistic antibodies. In brief, PBMCs were adjusted to 1E7 cells/ml in complete medium (RPMI 1640 Glutamax, 5% human serum, Sodium-Pyruvate, NEAA, 50 uM 2-Mercaptoethanol, Penicillin/Streptomycin) and cultured at 1.5 ml/well in a 24-well plate for 48 hours at 37° C., 5% CO₂. Cells were then re-harvested, washed in complete medium, centrifuged at 550×g for 5 min and adjusted with to the desired cell density required for functional characterization. To assess T cell proliferation, PBMCs were labelled with CFSE and CFSE-dilution was measured as proxy for T cell proliferation after 5 days of stimulation. In brief, cells were adjusted to 2×10⁷/ml in PBS and labelled with 2.5 μM CFSE proliferation dye (LifeTechnologies, Cat No 65-0850-84) for 6 minutes at 37° C., 5% CO₂. Cells were washed once in complete medium, followed by 2 washing steps in PBS. For stimulation with TGN1412, PBMCs were adjusted to 2×10⁶/ml in complete medium and 1×10⁵ cells were distributed to each well of a flat bottom 96-well plate and stimulated with increasing concentrations of TGN1412 (0.0002 nM to 10 nM, triplicates). CFSE-dilution was assessed by flow cytometry. Briefly, cells were centrifuged at 550×g for 5 min and washed with PBS. CFSE-dilution was assessed by flow cytometry. Briefly, cells were centrifuged at 550×g for 5 min and washed with PBS. Surface staining for CD8 (BV711 anti-human CD8a, BioLegend #301044), CD4 (PE-Cy7 anti-human CD4, BioLegend #344612) was performed according to the suppliers' indications. Cells were then washed twice with 150 μl/well PBS and resuspended in 200 μl/well FACS buffer and analyzed using BD FACS Fortessa. Cytokine secretion was measured at day 5 post activation via cytokine ELISA (huTNFα, DuoSet # DY210-05 and huIFNγ, DuoSet # DY285-05) or cytokine multiplex (Human Cytokine 17-plex assay, Bio-Rad # M5000031YV) analysis from culture supernatants.

Superagonism of CD28(SA) Requires FcγRIIb Cross-Linking

High Density Pre-Culture of PBMCs Restores CD28(SA) Superagonism

To understand the mechanism of action of CD28(SA), we validated high density (HD) pre-culture of PBMCs as a previously described protocol to restore the ability of PBMC-derived T cells to respond to TGN1412-mediated CD28 superagonism (Romer et al., 2011). As depicted in FIGS. 5A and 5B, CD28(SA) IgG4 (P1AE1975) induces PBMC T cell proliferation (FIG. 5A) and cytokine production (FIG. 5B) in a concentration-dependent manner at 5 days post stimulation only in PBMCs subjected to HD pre-culture, while fresh PBMCs remained unresponsive. We concluded that the previously published protocol to restore T cells' responsiveness to CD28(SA) in vitro (Romer et al., 2011) could be reproduced in our hands.

CD28(SA) Superagonistic Activity Requires Cross-Linking Via FcγRIIb—Blocking of FcγRIIb Abolishes CD28(SA) Functionality

Previously published literature indicates that TGN1412 potentially relies on FcγRIIb cross-linking. To understand the link between HD pre-culture of PBMCs and Fc-dependence of CD28(SA) functionality, the expression levels of FcγRIIb on PBMCs were assessed by flow cytometry before and after HD pre-culture. As depicted in FIG. 5C, FcγRIIb expression was absent in fresh PBMC monocytes, while 96.8% of monocytes expressed FcγRIIb after 2 days of HD pre-culture. Antibody-mediated blocking of FcγRIIb in subsequent T cell proliferation assays completely abrogated T cell proliferation upon stimulation with CD28(SA), measured after 5 days in culture (FIG. 5D). In an alternative approach, an Fc-silenced variant of CD28(SA) which carries the P329G-LALA mutation (CD28(SA) IgG1 PG-LALA: P1AD9289) did not display superagonistic function (FIG. 6A). These data confirm that CD28(SA)-mediated CD28 superagonism relies on cross-linking via FcγRIIb.

Adding a Tumor-Targeting Moiety for FAP-Targeting to Fc-Silent CD28(SA) Restores Superagonism, which is then Dependent on the Presence of the Tumor Target

Given that CD28 superagonism by CD28(SA) relies on FcγRIIb cross-linking, we hypothesized that FcR-dependence may be re-directed to tumors by introduction of (i) an Fc-silencing P329G-LALA mutation and (ii) a targeting moiety that cross-links to a surface-expressed tumor-antigen. To test this hypothesis, a FAP-targeting moiety was added as C-terminal fusion to an Fc-silenced CD28(SA) (FAP-CD28(SA) 1+2: P1AD9011). Since FcR-crosslinking was not required for this approach, PBMCs were not subjected to HD pre-culture. Instead, fresh PBMCs were co-cultured with 3T3-huFAP or 3T3-WT for 5 days in presence of increasing concentration of FAP-CD28 (P1AD9011) and T cell proliferation was assessed by CFSE-dilution via flow cytometry. As shown in FIG. 6B, the introduction of FAP-binding moiety enabled T cell proliferation exclusively in the presence of FAP. We concluded that superagonism can be selectively targeted to tumor antigens by Fc-silencing and addition of a tumor-targeting moiety.

In Vitro Assessment of T Cell Proliferation and Cytokine Secretion by Bispecific FAP-Targeted CD28 Antigen Binding Molecules in Absence and Presence of TCB Signals

Pan T cells were used as effector cells and isolated from PBMCs by MACS, using the Pan T Cell Isolation Kit (Miltenyi Biotec) according to the manufacturer's instructions.

To measure T cell activation by bispecific FAP-CD28 antigen binding molecules in absence of TCB, CFSE-labelled pan T cells were co-cultured with 3×10⁴/well 3T3-huFAP or parental 3T3 cells lacking FAP expression (3T3-WT), seeded the previous day in flat-bottom 96-well plates. Bispecific FAP-CD28 antigen binding molecules were added in increasing concentrations (0.0002 nM-10 nM, triplicates).

To measure T cell proliferation in presence of a TCB signal, CFSE-labelled pan T cells were incubated with 3×10⁴ FAP- and MCSP-expressing MV3 cells/well, seeded the previous day in flat-bottom 96-well plates, increasing concentrations of bispecific FAP-CD28 antigen binding molecules (0.0002 nM-10 nM, triplicates), and fixed concentration of MCSP-TCB (5 pM, P1AD2189). As controls, wells containing only TCB were included.

CFSE-dilution was assessed by flow cytometry and cytokine secretion was measured at 5 days post activation via cytokine ELISA (huTNFα, DuoSet # DY210-05 and huIFNγ, DuoSet # DY285-05) or cytokine multiplex (Human Cytokine 17-plex assay, Bio-Rad # M5000031YV) analysis from culture supernatants.

Conventional CD28 Agonistic Antibodies (Clone 9.3) do not Behave Superagonistically in Tumor-Targeted Bispecific Formats

Two types of CD28 agonistic antibodies have been reported in the literature: superagonistic CD28 antibodies such as TGN1412 are able to autonomously activate T cells without the necessity of an additional signal provided by TCR. These antibodies are referred to as superagonists, because they surpass the functionality of natural CD28 agonistic ligands CD80 and CD86, which strictly rely on the presence of a TCR signal to enhance T cell function. In contrast to superagonistic antibodies such as TGN1412, conventional agonistic antibodies such as clone mab 9.3 are not able to activate T cells autonomously, but, just like the natural CD28 ligands, require an additional TCR signal to enhance T cell activity. To assess the effect of targeting CD28 agonists to tumor antigens in more detail, we generated further FAP-CD28 molecules: (i) a superagonistic (SA) molecule with 2 CD28 binding moieties (TGN1412) and 2 FAP binding moieties=2+2 SA format (P1AD4493), (ii) a conventional agonist (CA) with 2 CD28 binding moieties (clone 9.3) and 1 or 2 FAP binding moieties, respectively: 2+2 CA (P1AD8968), 1+2 CA (P1AD8962). Fresh PBMCs were co-cultured with 3T3-huFAP or 3T3-WT for 5 days in presence of increasing concentration of the FAP-targeted molecules and T cell proliferation was assessed by CFSE-dilution via flow cytometry. As depicted in FIGS. 7A to 7D, only superagonistic binders were able to activate T cells. Further, T cell activation via the described superagonistic constructs is strictly dependent on the presence of FAP (FIG. 7B), as demonstrated by absent T cell activation in absence of FAP (FIG. 7D). In line with these data, also cytokine secretion was only observed for constructs harboring the superagonistic CD28(SA) antibodies, but not the conventional agonistic 9.3 antibody (FIG. 7E). We concluded that only superagonistic CD28 antibodies elicit autonomous T cell activation in bispecific tumor-targeted antibody formats, while the same formats with conventional 9.3 binders are not superagonistic.

### Example 6

### In Vitro Assessment of Tumor Cell Killing by Tumor-Targeted CD28 Molecules in the Absence or Presence of TCB

To assess the ability of bispecific FAP-CD28 or CEA-CD28 antigen binding molecules to achieve tumor cell killing or support TCB-mediated tumor cell killing, purified pan T cells served as effector cells and RFP-expressing MV3 cells and MKN45 cells, respectively, served as tumor targets.

To assess killing of MV3 tumor cells, 5000 MV3 target cells seeded the previous day were co-cultured with 1×10⁵ pan T cells per well in flat bottom 96-well plates (E:T 20:1), in presence of 5 pM MCSP-TCB (P1AD2189) alone or in combination with 10 nM bispecific FAP-CD28 antigen binding molecule. To assess killing of MV3 tumor cells, 5000 MV3 target cells seeded the previous day were co-cultured with 1×10⁵ pan T cells per well in flat bottom 96-well plates (E:T 20:1), in presence of 2 nM FAP-CD28. To assess the killing of MKN45 tumor cells, 5000 MKN45, seeded the previous day, were co-cultured with 1×10⁵ pan T cells per well in flat-bottom 96-well plates in presence of 2 nM CEA-CD28. Killing of target cells was monitored over the course of 90 hours, using the IncuCyte live cell imaging system (Essen Biosciences), capturing 4 images per well every 3 hours. RFP+ object counts per image (assessed via IncuCyte ZOOM software, Essen Biosciences) over time served as proxy for target cell death. Antibody-mediated target cell killing was distinguished from spontaneous target cell death by monitoring counts of target cells in presence of effector T cells alone over time (=baseline control). Killing was calculated as 100-x, x being % targets relative to the baseline control. Statistical analyses were performed using student's t-test, comparing the areas under the curves (AUC) of % killing over time.

FAP-CD28 Induces Target Cell Killing in the 2+1 Format, but Only with Superagonistic CD28 Binders, not with Conventional CD28 Agonistic Binders

The ability of FAP-CD28 molecules to induce tumor cell killing was assessed. As depicted in FIGS. 8A to 8D, co-culture of PBMC-derived T cells with FAP-expressing MV3 melanoma cells in presence of FAP-CD28 over 90 hours led to killing of MV3 cells exclusively by FAP CD28(SA) in 1+2 format (P1AD9011) and was comparable to the induction of killing achieved by a FAP-targeted TCB (P1AD4645). No killing was observed with FAP-CD28(SA) in 2+2 format (P1AD4493) as well as FAP-CD28 with conventional CD28 agonistic 9.3 antibody (P1AD8968 & P1AD8962). We conclude that in addition to T cell proliferation and cytokine secretion, a FAP-CD28 in 1+2 format with superagonistic binders can also elicit target cell killing, comparable to a TCB.

CEA-CD28 Induces Target Cell Killing in the 1+2 and 2+2 Format, but Only with Superagonistic Antibodies, not with Conventional CD28 Agonistic Antibodies

In an alternative approach, we used CEA-targeted CD28 agonistic molecules in the 2+2 SA (P1AE1195), 1+2 SA (P1AE1194), 2+2 CA (P1AE1193), and 2+1 CA (P1AE1192) formats to assess their ability to induce target cell killing. PBMC T cells were co-cultured with CEA-expressing MKN45 cells in presence of CEA-CD28 in the aforementioned formats for 90 h. Both formats containing superagonistic CD28 binders were able to induce killing of CEA-expressing MKN45 cells (FIGS. 9A and 9B). We speculate that the discrepancy between FAP-CD28(SA) 2+2 and CEA-CD28 (SA) 2+2's ability to kill their respective target cells lies within discrepancies of target expression levels in MKN45 vs. MV3 cells. Precisely, in house data confirmed that FAP-expression levels of MV3 cells are 10× lower than CEA-expression levels of MKN45 cells. Thus, in MV3 cells, tumor target binding sites might be limiting and killing of MV3 cells requires efficient occupancy of FAP vs. CD28, which is advantageous in the 1+2 format (i.e. 1 FAP binding site cross-links 2 CD28 binding sites) compared to the 2+2 (i.e. 2 FAP binding sites required for cross-linking of 2 CD28 binding sites).

CD28 Superagonism by TGN1412 Binders Relies on CD28 Binder Multivalency—Monovalent Binders are not Superagonistic

To further investigate the nature of CD28 superagonism, we assessed if monovalent CD28 TGN1412 binders display superagonistic behavior in a tumor-targeted bispecific format. PBMC T cells were co-cultured with 3T3-huFAP cells and incubated with increasing concentrations FAP-CD28 1+2 SA with CD28 bivalency (P1AD9011) and FAP-CD28 1+1 SA with CD28 monovalency (P1AD4492). As displayed in FIG. 10A, FAP-CD28 with monovalent CD28 binding (P1AD4492) was not able to induce T cell proliferation, as opposed to the CD28 bivalent construct (P1AD9011). Consistently, upregulation of the T cell activation markers CD69 and CD25 was only observed with the CD28 bivalency (FIGS. 10B and 10C, respectively). In conclusion, TGN1412-mediated superagonism does not only rely on cross-linking via Fc receptors but also requires CD28 binder multivalency.

In conclusion, it could be established that CD28 superagonism can be targeted specifically to tumor antigens by Fc-silencing and introduction of an antigen binding domain capable of specific binding to a tumor-associated antigen. Further, tumor-targeted bispecific antibodies are only superagonistic when they comprised CD28(SA)-based binders and not when they comprised conventional agonistic binders (clone 9.3). Further, superagonism requires multivalency of the CD28(SA) binder and monovalent CD28(SA) binding in bispecific constructs abrogates superagonistic T cell activation.

### REFERENCES

Acuto, O., and Michel, F. (2003). CD28-mediated co-stimulation: a quantitative support for TCR signalling. Nat Rev Immunol 3, 939-951.
Boomer, J. S., and Green, J. M. (2010). An enigmatic tail of CD28 signaling. Cold Spring Harb Perspect Biol 2, a002436.
Carreno, B. M., and Collins, M. (2002). The B7 family of ligands and its receptors: new pathways for costimulation and inhibition of immune responses. Annu Rev Immunol 20, 29-53.
Chen, L., and Flies, D. B. (2013). Molecular mechanisms of T cell co-stimulation and co-inhibition. Nat Rev Immunol 13, 227-242.
Engelhardt, J. J., Sullivan, T. J., and Allison, J. P. (2006). CTLA-4 overexpression inhibits T cell responses through a CD28-B7-dependent mechanism. J Immunol 177, 1052-1061.
Esensten, J. H., Helou, Y. A., Chopra, G., Weiss, A., and Bluestone, J. A. (2016). CD28 Costimulation: From Mechanism to Therapy. Immunity 44, 973-988.
Fraser, J. D., Irving, B. A., Crabtree, G. R., and Weiss, A. (1991). Regulation of interleukin-2 gene enhancer activity by the T cell accessory molecule CD28. Science 251, 313-316.
Hui, E., Cheung, J., Zhu, J., Su, X., Taylor, M. J., Wallweber, H. A., Sasmal, D. K., Huang, J., Kim, J. M., Mellman, I., and Vale, R. D. (2017). T cell costimulatory receptor CD28 is a primary target for PD-1-mediated inhibition. Science 355, 1428-1433.
Hunig, T. (2012). The storm has cleared: lessons from the CD28 superagonist TGN1412 trial. Nat Rev Immunol 12, 317-318.
June, C. H., Ledbetter, J. A., Gillespie, M. M., Lindsten, T., and Thompson, C. B. (1987). T-cell proliferation involving the CD28 pathway is associated with cyclosporine-resistant interleukin 2 gene expression. Mol Cell Biol 7, 4472-4481.
Kamphorst, A. O., Wieland, A., Nasti, T., Yang, S., Zhang, R., Barber, D. L., Konieczny, B. T., Daugherty, C. Z., Koenig, L., Yu, K., et al. (2017). Rescue of exhausted CD8 T cells by PD-1-targeted therapies is CD28-dependent. Science 355, 1423-1427.
Lavin, Y., Kobayashi, S., Leader, A., Amir, E. D., Elefant, N., Bigenwald, C., Remark, R., Sweeney, R., Becker, C. D., Levine, J. H., et al. (2017). Innate Immune Landscape in Early Lung Adenocarcinoma by Paired Single-Cell Analyses. Cell 169, 750-765 e717.
Linsley, P. S., Clark, E. A., and Ledbetter, J. A. (1990). T-cell antigen CD28 mediates adhesion with B cells by interacting with activation antigen B7/BB-1. Proc Natl Acad Sci USA 87, 5031-5035.
Romer, P. S., Berr, S., Avota, E., Na, S. Y., Battaglia, M., ten Berge, I., Einsele, H., and Hunig, T. (2011). Preculture of PBMCs at high cell density increases sensitivity of T-cell responses, revealing cytokine release by CD28 superagonist TGN1412. Blood 118, 6772-6782.
Tai, X., Van Laethem, F., Sharpe, A. H., and Singer, A. (2007). Induction of autoimmune disease in CTLA-4−/− mice depends on a specific CD28 motif that is required for in vivo costimulation. Proc Natl Acad Sci USA 104, 13756-13761.
Thompson, C. B., Lindsten, T., Ledbetter, J. A., Kunkel, S. L., Young, H. A., Emerson, S. G., Leiden, J. M., and June, C. H. (1989). CD28 activation pathway regulates the production of multiple T-cell-derived lymphokines/cytokines. Proc Natl Acad Sci USA 86, 1333-1337.
Tirosh, I., Izar, B., Prakadan, S. M., Wadsworth, M. H., 2nd, Treacy, D., Trombetta, J. J., Rotem, A., Rodman, C., Lian, C., Murphy, G., et al. (2016). Dissecting the multicellular ecosystem of metastatic melanoma by single-cell RNA-seq. Science 352, 189-196.
Zheng, C., Zheng, L., Yoo, J. K., Guo, H., Zhang, Y., Guo, X., Kang, B., Hu, R., Huang, J. Y., Zhang, Q., et al. (2017). Landscape of Infiltrating T Cells in Liver Cancer Revealed by Single-Cell Sequencing. Cell 169, 1342-1356 e1316.

1. A superagonistic CD28 antigen binding molecule, which is capable of bivalent binding to CD28 and comprises
(a) two or more antigen binding domains capable of specific binding to CD28,
(b) at least one antigen binding domain capable of specific binding to a tumor-associated antigen, and
(c) an Fc domain composed of a first and a second subunit capable of stable association comprising one or more amino acid substitution that reduces the binding affinity of the antigen binding molecule to an Fc receptor and/or effector function.

2. The superagonistic CD28 antigen binding molecule of claim 1, wherein the Fc domain is of human IgG1 subclass and comprises the amino acid mutations L234A, L235A and P329G (numbering according to Kabat EU index).

3. The superagonistic CD28 antigen binding molecule of claim 1 or 2, wherein each of the antigen binding domains capable of specific binding to CD28 comprises
(i) a heavy chain variable region (V_{H}CD28) comprising a heavy chain complementary determining region CDR-H1 of SEQ ID NO: 20, a CDR-H2 of SEQ ID NO: 21, and a CDR-H3 of SEQ ID NO: 22, and a light chain variable region (V_{L}CD28) comprising a light chain complementary determining region CDR-L1 of SEQ ID NO: 23, a CDR-L2 of SEQ ID NO: 24 and a CDR-L3 of SEQ ID NO: 25; or
(ii) a heavy chain variable region (V_{H}CD28) comprising a CDR-H1 of SEQ ID NO: 36, a CDR-H2 of SEQ ID NO: 37, and a CDR-H3 of SEQ ID NO: 38, and a light chain variable region (V_{L}CD28) comprising a CDR-L1 of SEQ ID NO: 39, a CDR-L2 of SEQ ID NO: 40 and a CDR-L3 of SEQ ID NO: 41.

4. The superagonistic CD28 antigen binding molecule of any one of claims 1 to 3, wherein each of the antigen binding domains capable of specific binding to CD28 comprises a heavy chain variable region (V_{H}CD28) comprising a CDR-H1 of SEQ ID NO: 20, a CDR-H2 of SEQ ID NO: 21, and a CDR-H3 of SEQ ID NO: 22, and a light chain variable region (V_{L}CD28) comprising a CDR-L1 of SEQ ID NO: 23, a CDR-L2 of SEQ ID NO: 24 and a CDR-L3 of SEQ ID NO: 25.

5. The superagonistic CD28 antigen binding molecule of any one of claims 1 to 4, wherein each of the antigen binding domains capable of specific binding to CD28 comprises a heavy chain variable region (V_{H}CD28) comprising an amino acid sequence that is at least about 95%, 96%, 97%, 98%, 99% or 100% identical to the amino acid sequence of SEQ ID NO:26, and a light chain variable region (V_{L}CD28) comprising an amino acid sequence that is at least about 95%, 96%, 97%, 98%, 99% or 100% identical to the amino acid sequence of SEQ ID NO:27.

6. The superagonistic CD28 antigen binding molecule of any one of claims 1 to 3, wherein each of the antigen binding domains capable of specific binding to CD28 comprises a heavy chain variable region (V_{H}CD28) comprising an amino acid sequence selected from the group consisting of SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50 and SEQ ID NO:51, and a light chain variable region (V_{L}CD28) comprising an amino acid sequence selected from the group consisting of SEQ ID NO:27, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60 and SEQ ID NO:61.

7. The superagonistic CD28 antigen binding molecule of any one of claim 1 to 3 or 6, wherein each of the antigen binding domains capable of specific binding to CD28 comprises
(a) a heavy chain variable region (V_{H}CD28) comprising the amino acid sequence of SEQ ID NO:47 and a light chain variable region (V_{L}CD28) comprising the amino acid sequence of SEQ ID NO:54, or
(b) a heavy chain variable region (V_{H}CD28) comprising the amino acid sequence of SEQ ID NO:47 and a light chain variable region (V_{L}CD28) comprising the amino acid sequence of SEQ ID NO:27, or
(c) a heavy chain variable region (V_{H}CD28) comprising the amino acid sequence of SEQ ID NO:51 and a light chain variable region (V_{L}CD28) comprising the amino acid sequence of SEQ ID NO:61, or
(d) a heavy chain variable region (V_{H}CD28) comprising the amino acid sequence of SEQ ID NO:46 and a light chain variable region (V_{L}CD28) comprising the amino acid sequence of SEQ ID NO:53, or
(e) a heavy chain variable region (V_{H}CD28) comprising the amino acid sequence of SEQ ID NO:46 and a light chain variable region (V_{L}CD28) comprising the amino acid sequence of SEQ ID NO:54, or
(f) a heavy chain variable region (V_{H}CD28) comprising the amino acid sequence of SEQ ID NO:46 and a light chain variable region (V_{L}CD28) comprising the amino acid sequence of SEQ ID NO:59, or
(g) a heavy chain variable region (V_{H}CD28) comprising the amino acid sequence of SEQ ID NO:46 and a light chain variable region (V_{L}CD28) comprising the amino acid sequence of SEQ ID NO:27, or
(h) a heavy chain variable region (V_{H}CD28) comprising the amino acid sequence of SEQ ID NO:43 and a light chain variable region (V_{L}CD28) comprising the amino acid sequence of SEQ ID NO:27, or
(i) a heavy chain variable region (V_{H}CD28) comprising the amino acid sequence of SEQ ID NO:42 and a light chain variable region (V_{L}CD28) comprising the amino acid sequence of SEQ ID NO:53, or
(j) a heavy chain variable region (V_{H}CD28) comprising the amino acid sequence of SEQ ID NO:42 and a light chain variable region (V_{L}CD28) comprising the amino acid sequence of SEQ ID NO:59, or
(k) a heavy chain variable region (V_{H}CD28) comprising the amino acid sequence of SEQ ID NO:42 and a light chain variable region (V_{L}CD28) comprising the amino acid sequence of SEQ ID NO:27.

8. The superagonistic CD28 antigen binding molecule of any one of claims 1 to 7, wherein each of the antigen binding domains capable of specific binding to CD28 is a Fab fragment.

9. The superagonistic CD28 antigen binding molecule of any one of claims 1 to 8, wherein the antigen binding domain capable of specific binding to a tumor-associated antigen is an antigen binding domain capable of specific binding to Carcinoembryonic Antigen (CEA).

10. The superagonistic CD28 antigen binding molecule of any one of claims 1 to 9, wherein the antigen binding domain capable of specific binding to CEA comprises a heavy chain variable region (V_{H}CEA) comprising (i) CDR-H1 comprising the amino acid sequence of SEQ ID NO:127, (ii) CDR-H2 comprising the amino acid sequence of SEQ ID NO:128, and (iii) CDR-H3 comprising the amino acid sequence of SEQ ID NO:129, and a light chain variable region (V_{L}CEA) comprising (iv) CDR-L1 comprising the amino acid sequence of SEQ ID NO:130, (v) CDR-L2 comprising the amino acid sequence of SEQ ID NO:131, and (vi) CDR-L3 comprising the amino acid sequence of SEQ ID NO:132.

11. The superagonistic CD28 antigen binding molecule of any one of claims 1 to 10, wherein the antigen binding domain capable of specific binding to CEA comprises a heavy chain variable region (V_{H}CEA) comprising an amino acid sequence that is at least about 95%, 96%, 97%, 98%, 99% or 100% identical to the amino acid sequence of SEQ ID NO:133, and a light chain variable region (V_{L}CEA) comprising an amino acid sequence that is at least about 95%, 96%, 97%, 98%, 99% or 100% identical to the amino acid sequence of SEQ ID NO:134.

12. The superagonistic CD28 antigen binding molecule of any one of claims 1 to 8, wherein the antigen binding domain capable of specific binding to a tumor-associated antigen is an antigen binding domain capable of specific binding to Fibroblast Activation Protein (FAP).

13. The superagonistic CD28 antigen binding molecule of any one of claim 1 to 8 or 12, wherein the antigen binding domain capable of specific binding to FAP comprises
(a) a heavy chain variable region (V_{H}FAP) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}FAP) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17, or
(b) a heavy chain variable region (V_{H}FAP) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}FAP) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9.

14. The superagonistic CD28 antigen binding molecule of any one of claim 1 to 8 or 12 or 13, wherein the antigen binding domain capable of specific binding to FAP comprises
(a) a heavy chain variable region (V_{H}FAP) comprising an amino acid sequence that is at least about 95%, 96%, 97%, 98%, 99% or 100% identical to the amino acid sequence of SEQ ID NO:18, and a light chain variable region (V_{L}FAP) comprising an amino acid sequence that is at least about 95%, 96%, 97%, 98%, 99% or 100% identical to the amino acid sequence of SEQ ID NO:19, or
(b) a heavy chain variable region (V_{H}FAP) comprising an amino acid sequence that is at least about 95%, 96%, 97%, 98%, 99% or 100% identical to the amino acid sequence of SEQ ID NO:10, and a light chain variable region (V_{L}FAP) comprising an amino acid sequence that is at least about 95%, 96%, 97%, 98%, 99% or 100% identical to the amino acid sequence of SEQ ID NO:11.

15. The superagonistic CD28 antigen binding molecule of any one of claims 1 to 14, comprising
(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and
(b) a VH and VL domain capable of specific binding to a tumor-associated antigen, wherein the VH domain is connected via a peptide linker to the C-terminus of one of the two heavy chains and wherein the VL domain is connected via a peptide linker to the C-terminus of the second heavy chain.

16. The superagonistic CD28 antigen binding molecule of any one of claims 1 to 14, comprising
(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and
(b) a crossFab fragment capable of specific binding to a tumor-associated antigen which is connected via a peptide linker to the C-terminus of one of the two heavy chains.

17. The superagonistic CD28 antigen binding molecule of any one of claims 1 to 14, comprising
(a) two light chains and two heavy chains of an antibody comprising two Fab fragments capable of specific binding to CD28 and the Fc domain comprising one or more amino acid substitution that reduces the binding affinity of the antigen binding molecule to an Fc receptor and/or effector function, and
(b) two crossFab fragments capable of specific binding to a tumor-associated antigen, wherein one crossFab fragment is connected via a peptide linker to the C-terminus of one of the two heavy chains and wherein the other crossFab fragment is connected via a peptide linker to the C-terminus of the second heavy chain.

18. A polynucleotide encoding the bispecific antigen binding molecule of any one of paras 1 to 17.

19. A host cell comprising the polynucleotide of claim 18.

20. A method of producing the superagonistic CD28 antigen binding molecule of any one of claims 1 to 17 comprising culturing the host cell of claim 19 under conditions suitable for the expression of the bispecific antigen binding molecule.

21. A pharmaceutical composition comprising superagonistic CD28 antigen binding molecule of any one of claims 1 to 17 and at least one pharmaceutically acceptable excipient.

22. The superagonistic CD28 antigen binding molecule of any one of claims 1 to 17, or the pharmaceutical composition of claim 21, for use as a medicament.

23. The superagonistic CD28 antigen binding molecule of any one of claims 1 to 17, or the pharmaceutical composition of claim 21, for use in the treatment of cancer.

24. The superagonistic CD28 antigen binding molecule of any one of claims 1 to 17 for use in the treatment of cancer, wherein the superagonistic CD28 antigen binding molecule is administered in combination with a chemotherapeutic agent, radiation therapy and/or other agents for use in cancer immunotherapy.

25. Use of the superagonistic CD28 antigen binding molecule of any one of claims 1 to 17, or the pharmaceutical composition of claim 21, in the manufacture of a medicament for the treatment of cancer.

26. A method of inhibiting the growth of tumor cells in an individual comprising administering to the individual an effective amount of the superagonistic CD28 antigen binding molecule of any one of claims 1 to 17, or the pharmaceutical composition of claim 22, to inhibit the growth of the tumor cells.

27. A method of treating cancer comprising administering to the individual a therapeutically effective amount of the superagonistic CD28 antigen binding molecule of any one of claims 1 to 20, or the pharmaceutical composition of claim 24.


SEQUENCE LISTING (162 sequences)

SEQ ID NO: 1 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 3 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 4 - protein, 5 aa, Artificial Sequence
  FAP(28H1)  CDR-H1
SHAMS

SEQ ID NO: 5 - protein, 16 aa, Artificial Sequence
  FAP(28H1)  CDR-H2
AIWASGEQYYADSVKG

SEQ ID NO: 6 - protein, 8 aa, Artificial Sequence
  FAP(28H1)  CDR-H3
GWLGNFDY

SEQ ID NO: 7 - protein, 12 aa, Artificial Sequence
  FAP(28H1)  CDR-L1
RASQSVSRSYLA

SEQ ID NO: 8 - protein, 7 aa, Artificial Sequence
  FAP(28H1)  CDR-L2
GASTRAT

SEQ ID NO: 9 - protein, 9 aa, Artificial Sequence
  FAP(28H1)  CDR-L3
QQGQVIPPT

SEQ ID NO: 10 - protein, 116 aa, Artificial Sequence
  FAP(28H1) VH
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 11 - protein, 108 aa, Artificial Sequence
  FAP(28H1) VL
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 12 - protein, 5 aa, Artificial Sequence
  FAP(4B9)  CDR-H1
SYAMS

SEQ ID NO: 13 - protein, 17 aa, Artificial Sequence
  FAP(4B9)  CDR-H2
AIIGSGASTYYADSVKG

SEQ ID NO: 14 - protein, 8 aa, Artificial Sequence
  FAP(4B9)  CDR-H3
GWFGGFNY

SEQ ID NO: 15 - protein, 12 aa, Artificial Sequence
  FAP(4B9)  CDR-L1
RASQSVSRSYLA

SEQ ID NO: 16 - protein, 7 aa, Artificial Sequence
  FAP(4B9)  CDR-L2
VGSRRAT

SEQ ID NO: 17 - protein, 9 aa, Artificial Sequence
  FAP(4B9)  CDR-L3
QQGIMLPPT

SEQ ID NO: 18 - protein, 117 aa, Artificial Sequence
  FAP(4B9)  VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 19 - protein, 108 aa, Artificial Sequence
  FAP(4B9)  VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 20 - protein, 5 aa, Artificial Sequence
  CD28(SA) CDR-H1
SYYIH

SEQ ID NO: 21 - protein, 17 aa, Artificial Sequence
  CD28(SA) CDR-H2
CIYPGNVNTNYNEKFKD

SEQ ID NO: 22 - protein, 11 aa, Artificial Sequence
  CD28(SA) CDR-H3
SHYGLDWNFDV

SEQ ID NO: 23 - protein, 11 aa, Artificial Sequence
  CD28(SA) CDR-L1
HASQNIYVWLN

SEQ ID NO: 24 - protein, 7 aa, Artificial Sequence
  CD28(SA) CDR-L2
KASNLHT

SEQ ID NO: 25 - protein, 9 aa, Artificial Sequence
  CD28(SA) CDR-L3
QQGQTYPYT

SEQ ID NO: 26 - protein, 120 aa, Artificial Sequence
  CD28(SA) VH
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 27 - protein, 107 aa, Artificial Sequence
  CD28(SA) VL
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 28 - protein, 5 aa, Artificial Sequence
  CD28(mAb 9.3) CDR-H1
DYGVH

SEQ ID NO: 29 - protein, 16 aa, Artificial Sequence
  CD28(mAb 9.3) CDR-H2
VIWAGGGTNYNSALMS

SEQ ID NO: 30 - protein, 12 aa, Artificial Sequence
  CD28(mAb 9.3) CDR-H3
DKGYSYYYSMDY

SEQ ID NO: 31 - protein, 15 aa, Artificial Sequence
  CD28(mAb 9.3) CDR-L1
RASESVEYYVTSLMQ

SEQ ID NO: 32 - protein, 7 aa, Artificial Sequence
  CD28(mAb 9.3) CDR-L2
AASNVES

SEQ ID NO: 33 - protein, 9 aa, Artificial Sequence
  CD28(mAb 9.3) CDR-L3
QQSRKVPYT

SEQ ID NO: 34 - protein, 120 aa, Artificial Sequence
  CD28(mAb 9.3) VH
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSS

SEQ ID NO: 35 - protein, 111 aa, Artificial Sequence
  CD28(mAb 9.3) VL
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIK

SEQ ID NO: 36 - protein, 5 aa, Artificial Sequence
  CD28 CDR-H1 consensus
SYYIH

SEQ ID NO: 37 - protein, 17 aa, Artificial Sequence
  CD28 CDR-H2 consensus
  VARIANT (5)..(5): Gly or Arg
  VARIANT (6)..(6): Asn or Asp
  VARIANT (7)..(7): Val or Gly
  VARIANT (8)..(8): Asn, Gln or Ala
SIYPXXXXTNYNEKFKD

SEQ ID NO: 38 - protein, 11 aa, Artificial Sequence
  CD28 CDR-H3 consensus
  VARIANT (5)..(5): Leu or Ala
  VARIANT (7)..(7): Trp, His, Tyr or Phe
SHYGXDXNFDV

SEQ ID NO: 39 - protein, 11 aa, Artificial Sequence
  CD28 CDR-L1 consensus
  VARIANT (1)..(1): His or Arg
  VARIANT (5)..(5): Asn or Gly
  VARIANT (7)..(7): Tyr or Ser
  VARIANT (8)..(8): Val or Asn
  VARIANT (9)..(9): Trp, His, Phe or Tyr
XASQXIXXXLN

SEQ ID NO: 40 - protein, 7 aa, Artificial Sequence
  CD28 CDR-L2 consensus
  VARIANT (1)..(1): Lys or Tyr
  VARIANT (2)..(2): Ala or Gly
  VARIANT (4)..(4): Asn or Ser
  VARIANT (6)..(6): His or Tyr
  VARIANT (7)..(7): Thr or Ser
XXSXLXX

SEQ ID NO: 41 - protein, 9 aa, Artificial Sequence
  CD28 CDR-L3 consensus
  VARIANT (3)..(3): Gly or Ala
QQXQTYPYT

SEQ ID NO: 42 - protein, 120 aa, Artificial Sequence
  CD28 VH variant a
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSS

SEQ ID NO: 43 - protein, 120 aa, Artificial Sequence
  CD28 VH variant b
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDHNFDVWGQGTTVTVSS

SEQ ID NO: 44 - protein, 120 aa, Artificial Sequence
  CD28 VH variant c
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGADHNFDVWGQGTTVTVSS

SEQ ID NO: 45 - protein, 120 aa, Artificial Sequence
  CD28 VH variant d
QVQLVQSGAEVKKPGASVKVSCKASGYTFTSYYIHWVRQAPGQGLEWIGSIYPRDGQTNYNEKFKDRATLTVDTSISTAYMELSRLRSDDTAVYFCTRSHYGLDYNFDVWGQGTTVTVSS

SEQ ID NO: 46 - protein, 120 aa, Artificial Sequence
  CD28 VH variant e
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDWNFDVWGQGTTVTVSS

SEQ ID NO: 47 - protein, 120 aa, Artificial Sequence
  CD28 VH variant f
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDFNFDVWGQGTTVTVSS

SEQ ID NO: 48 - protein, 120 aa, Artificial Sequence
  CD28 VH variant g
QVQLVQSGAEVKKPGASVKVSCKASGYTFTSYYIHWVRQAPGQGLEWIGSIYPRNVQTNYNEKFKDRATLTVDTSISTAYMELSRLRSDDTAVYFCTRSHYGLDHNFDVWGQGTTVTVSS

SEQ ID NO: 49 - protein, 120 aa, Artificial Sequence
  CD28 VH variant h
QVQLVQSGAEVKKPGASVKVSCKASGYTFTSYYIHWVRQAPGQGLEWIGSIYPRDVQTNYNEKFKDRATLTVDTSISTAYMELSRLRSDDTAVYFCTRSHYGLDHNFDVWGQGTTVTVSS

SEQ ID NO: 50 - protein, 120 aa, Artificial Sequence
  CD28 VH variant i
EVQLVESGGGLVQPGGSLRLSCAASGFTFTSYYIHWVRQAPGKGLEWVASIYPGNVNTRYADSVKGRFTISADTSKNTAYLQMNSLRAEDTAVYYCTRSHYGLDWNFDVWGQGTTVTVSS

SEQ ID NO: 51 - protein, 120 aa, Artificial Sequence
  CD28 VH variant j
EVQLVESGGGLVQPGGSLRLSCAASGFTFTSYYIHWVRQAPGKGLEWVASIYPGNVATRYADSVKGRFTISADTSKNTAYLQMNSLRAEDTAVYYCTRSHYGLDWNFDVWGQGTTVTVSS

SEQ ID NO: 52 - protein, 107 aa, Artificial Sequence
  CD28 VL variant k
DIQMTQSPSSLSASVGDRVTITCHASQNIYVHLNWYQQKPGKAPKLLIYKASNLHTGVPSRFSGSGSGTDFTLTISSLQPEDFATYYCQQAQTYPYTFGGGTKVEIK

SEQ ID NO: 53 - protein, 107 aa, Artificial Sequence
  CD28 VL variant l
DIQMTQSPSSLSASVGDRVTITCHASQNIYVFLNWYQQKPGKAPKLLIYKASNLHTGVPSRFSGSGSGTDFTLTISSLQPEDFATYYCQQGQTYPYTFGGGTKVEIK

SEQ ID NO: 54 - protein, 107 aa, Artificial Sequence
  CD28 VL variant m
DIQMTQSPSSLSASVGDRVTITCHASQNIYVYLNWYQQKPGKAPKLLIYKASNLHTGVPSRFSGSGSGTDFTLTISSLQPEDFATYYCQQGQTYPYTFGGGTKVEIK

SEQ ID NO: 55 - protein, 107 aa, Artificial Sequence
  CD28 VL variant n
DIQMTQSPSSLSASVGDRVTITCHASQGISNYLNWYQQKPGKAPKLLIYKASNLHTGVPSRFSGSGSGTDFTLTISSLQPEDFATYYCQQGQTYPYTFGGGTKVEIK

SEQ ID NO: 56 - protein, 107 aa, Artificial Sequence
  CD28 VL variant o
DIQMTQSPSSLSASVGDRVTITCHASQNIYVWLNWYQQKPGKAPKLLIYYTSSLHSGVPSRFSGSGSGTDFTLTISSLQPEDFATYYCQQGQTYPYTFGGGTKVEIK

SEQ ID NO: 57 - protein, 107 aa, Artificial Sequence
  CD28 VL variant p
DIQMTQSPSSLSASVGDRVTITCHASQGISNYLNWYQQKPGKAPKLLIYYTSSLHSGVPSRFSGSGSGTDFTLTISSLQPEDFATYYCQQGQTYPYTFGGGTKVEIK

SEQ ID NO: 58 - protein, 107 aa, Artificial Sequence
  CD28 VL variant q
DIQMTQSPSSLSASVGDRVTITCHASQGISNHLNWYQQKPGKAPKLLIYKASNLHTGVPSRFSGSGSGTDFTLTISSLQPEDFATYYCQQGQTYPYTFGGGTKVEIK

SEQ ID NO: 59 - protein, 107 aa, Artificial Sequence
  CD28 VL variant r
DIQMTQSPSSLSASVGDRVTITCHASQGIYVYLNWYQQKPGKAPKLLIYKASNLHTGVPSRFSGSGSGTDFTLTISSLQPEDFATYYCQQGQTYPYTFGGGTKVEIK

SEQ ID NO: 60 - protein, 107 aa, Artificial Sequence
  CD28 VL variant s
DIQMTQSPSSLSASVGDRVTITCHASQGISVYLNWYQQKPGKAPKLLIYKASNLHTGVPSRFSGSGSGTDFTLTISSLQPEDFATYYCQQGQTYPYTFGGGTKVEIK

SEQ ID NO: 61 - protein, 107 aa, Artificial Sequence
  CD28 VL variant t
DIQMTQSPSSLSASVGDRVTITCRASQNIYVWLNWYQQKPGKAPKLLIYKASNLYSGVPSRFSGSRSGTDFTLTISSLQPEDFATYYCQQGQTYPYTFGQGTKLEIK

SEQ ID NO: 62 - protein, 214 aa, Artificial Sequence
  CD28(SA) light chain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 447 aa, Artificial Sequence
  CD28(SA) hu IgG4 heavy chain
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 448 aa, Artificial Sequence
  CD28(SA) hu IgG1 PGLALA heavy chain
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 65 - protein, 214 aa, Artificial Sequence
  CD28(SA)  hu IgG light chain “RK”
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 448 aa, Artificial Sequence
  CD28(SA) hu IgG1 PGLALA Fc knob
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 67 - protein, 438 aa, Artificial Sequence
  FAP(4B9) VL-CH hu IgG1 PGLALA Fc hole
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 68 - protein, 224 aa, Artificial Sequence
  FAP(4B9) VH-Ckappa
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 820 aa, Artificial Sequence
  CD28(SA) VHCH-VHCH Fc knob FAP(4B9) VH PGLALA
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 70 - protein, 811 aa, Artificial Sequence
  CD28(SA) VHCH-VHCH Fc hole FAP(4B9) VL PGLALA
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 71 - protein, 586 aa, Artificial Sequence
  CD28(SA) VHCH- Fc knob FAP(4B9) VH
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 72 - protein, 577 aa, Artificial Sequence
  CD28(SA) VHCH- Fc hole FAP(4B9) VL
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 73 - protein, 693 aa, Artificial Sequence
  CD28(SA) VHCH “EE”- Fc PGLALA FAP(4B9) VHCL
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 214 aa, Artificial Sequence
  FAP(4B9) VLCH1
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 75 - protein, 668 aa, Artificial Sequence
  CD28(SA) VLCH1- FAP(4B9) VHCH1 “EE”- Fc knob PGLALA
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 76 - protein, 445 aa, Artificial Sequence
  FAP(4B9) VHCH1 “EE”- Fc hole PGLALA
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 77 - protein, 227 aa, Artificial Sequence
  CD28(SA) VHCL
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 215 aa, Artificial Sequence
  FAP(4B9) VLCL “RK”
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 225 aa, Artificial Sequence
  Fc hole PGLALA
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 80 - protein, 363 aa, Artificial Sequence
  Fc knob -FAP(4B9) VH
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 81 - protein, 697 aa, Artificial Sequence
  CD28(SA) VHCH1 “EE”- Fc PGLALA CEA(Medi-565) VHCL
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 221 aa, Artificial Sequence
  CEA-VLCH1
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 83 - protein, 590 aa, Artificial Sequence
  CD28(SA) VHCH1- Fc knob CEA VH
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 84 - protein, 585 aa, Artificial Sequence
  CD28(SA) VHCH1- Fc hole CEA VL
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 85 - protein, 448 aa, Artificial Sequence
  CD28(SA) VHCH1 “EE”- Fc hole PGLALA HYRF
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSP

SEQ ID NO: 86 - protein, 225 aa, Artificial Sequence
  Fc knob PGLALA
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 87 - protein, 446 aa, Artificial Sequence
  CEA VL-CH hu IgG1 PGLALA Fc hole
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 88 - protein, 228 aa, Artificial Sequence
  CEA VH-CL
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 218 aa, Artificial Sequence
  CD28(mAb 9.3) light chain
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 448 aa, Artificial Sequence
  CD28(mAb 9.3) hu IgG1 PGLALA heavy chain
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 91 - protein, 448 aa, Artificial Sequence
  CD28(mAb 9.3) hu IgG1 PGLALA Fc knob “EE”
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 92 - protein, 218 aa, Artificial Sequence
  CD28(mAb 9.3) hu IgG light chain “RK”
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 820 aa, Artificial Sequence
  CD28(mAb 9.3) VHCH-VHCH Fc knob FAP(4B9) VH PGLALA
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 94 - protein, 811 aa, Artificial Sequence
  CD28(mAb 9.3) VHCH-VHCH Fc hole FAP(4B9) VL PGLALA
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 95 - protein, 586 aa, Artificial Sequence
  CD28(mAb 9.3) VHCH- Fc knob FAP(4B9) VH
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 96 - protein, 577 aa, Artificial Sequence
  CD28(mAb 9.3) VHCH- Fc hole FAP(4B9) VL
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 97 - protein, 693 aa, Artificial Sequence
  CD28(mAb 9.3) VHCH “EE”- Fc PGLALA FAP(4B9) VHCL
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 218 aa, Artificial Sequence; identical to SEQ ID NO: 92
  CD28(mAb 9.3) VLCL “RK”

SEQ ID NO: 99 - protein, 213 aa, Artificial Sequence
  FAP(4B9) VL-CH1
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 100 - protein, 672 aa, Artificial Sequence
  CD28(mAb 9.3) VLCH1- FAP(4B9) VHCH1 “EE”- Fc knob PGLALA
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 101 - protein, 227 aa, Artificial Sequence
  CD28(mAb 9.3) VHCL
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 697 aa, Artificial Sequence
  CD28(mAb 9.3) VHCH1 “EE”- Fc PGLALA CEA VHCL
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 590 aa, Artificial Sequence
  CD28(mAb 9.3) VHCH1- Fc knob CEA VH
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 104 - protein, 585 aa, Artificial Sequence
  CD28(mAb 9.3) VHCH1- Fc hole CEA VL
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 105 - protein, 448 aa, Artificial Sequence
  CD28(mAb 9.3) VHCH1 “EE”- Fc hole PGLALA HYRF
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 106 - protein, 218 aa, Artificial Sequence; identical to SEQ ID NO: 92
  CD28(mAb 9.3) VLCL “RK”

SEQ ID NO: 107 - protein, 834 aa, Artificial Sequence
  CD28(SA) VHCH1 “EE” Fc hole PGLALA FAP(4B9) VH - CEA(Medi-565) VHCL
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSGGGGSGGGGSGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 577 aa, Artificial Sequence
  CD28(SA) VHCH1 “EE” Fc knob PGLALA FAP(4B9) VL
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 109 - protein, 221 aa, Artificial Sequence; identical to SEQ ID NO: 82
  CEA VLCH1

SEQ ID NO: 110 - protein, 727 aa, Artificial Sequence
  CD28(SA) VHCH1 Fc hole PGLALA FAP(4B9) VH - CEA VH
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSGGGGSGGGGSGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 111 - protein, 713 aa, Artificial Sequence
  CD28(SA) VHCH1 Fc knob PGLALA FAP(4B9) VL - CEA VL
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKGGGGSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 112 - protein, 448 aa, Artificial Sequence; identical to SEQ ID NO: 66
  VH (CD28 parental) CH1- Fc knob PGLALA

SEQ ID NO: 113 - protein, 448 aa, Artificial Sequence
  VH (CD28 variant g) CH1- Fc knob PGLALA
QVQLVQSGAEVKKPGASVKVSCKASGYTFTSYYIHWVRQAPGQGLEWIGSIYPRNVQTNYNEKFKDRATLTVDTSISTAYMELSRLRSDDTAVYFCTRSHYGLDHNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 114 - protein, 448 aa, Artificial Sequence
  VH (CD28 variant f) CH1- Fc knob PGLALA
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDFNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 115 - protein, 448 aa, Artificial Sequence
  VH (CD28 variant j) CH1- Fc knob PGLALA
EVQLVESGGGLVQPGGSLRLSCAASGFTFTSYYIHWVRQAPGKGLEWVASIYPGNVATRYADSVKGRFTISADTSKNTAYLQMNSLRAEDTAVYYCTRSHYGLDWNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 116 - protein, 448 aa, Artificial Sequence
  VH (CD28 variant e) CH1- Fc knob PGLALA
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDWNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 117 - protein, 448 aa, Artificial Sequence
  VH (CD28 variant b) CH1- Fc knob PGLALA
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDHNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 118 - protein, 448 aa, Artificial Sequence
  VH (CD28 variant a) CH1- Fc knob PGLALA
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 119 - protein, 448 aa, Artificial Sequence
  VH (CD28 variant i) CH1- Fc knob PGLALA
EVQLVESGGGLVQPGGSLRLSCAASGFTFTSYYIHWVRQAPGKGLEWVASIYPGNVNTRYADSVKGRFTISADTSKNTAYLQMNSLRAEDTAVYYCTRSHYGLDWNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 120 - protein, 214 aa, Artificial Sequence
  VL (CD28 variant k)-CL
DIQMTQSPSSLSASVGDRVTITCHASQNIYVHLNWYQQKPGKAPKLLIYKASNLHTGVPSRFSGSGSGTDFTLTISSLQPEDFATYYCQQAQTYPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 214 aa, Artificial Sequence
  VL (CD28 variant l)-CL
DIQMTQSPSSLSASVGDRVTITCHASQNIYVFLNWYQQKPGKAPKLLIYKASNLHTGVPSRFSGSGSGTDFTLTISSLQPEDFATYYCQQGQTYPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 214 aa, Artificial Sequence
  VL (CD28 variant m)-CL
DIQMTQSPSSLSASVGDRVTITCHASQNIYVYLNWYQQKPGKAPKLLIYKASNLHTGVPSRFSGSGSGTDFTLTISSLQPEDFATYYCQQGQTYPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 214 aa, Artificial Sequence
  VL (CD28 variant r)-CL
DIQMTQSPSSLSASVGDRVTITCHASQGIYVYLNWYQQKPGKAPKLLIYKASNLHTGVPSRFSGSGSGTDFTLTISSLQPEDFATYYCQQGQTYPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 214 aa, Artificial Sequence
  VL (CD28 variant s)-CL
DIQMTQSPSSLSASVGDRVTITCHASQGISVYLNWYQQKPGKAPKLLIYKASNLHTGVPSRFSGSGSGTDFTLTISSLQPEDFATYYCQQGQTYPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 214 aa, Artificial Sequence
  VL (CD28 variant t)-CL
DIQMTQSPSSLSASVGDRVTITCRASQNIYVWLNWYQQKPGKAPKLLIYKASNLYSGVPSRFSGSRSGTDFTLTISSLQPEDFATYYCQQGQTYPYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 225 aa, Artificial Sequence
  Fc hole PGLALA, HYRF
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSP

SEQ ID NO: 127 - protein, 5 aa, Artificial Sequence
  CEA CDR-H1
SYWMH

SEQ ID NO: 128 - protein, 19 aa, Artificial Sequence
  CEA CDR-H2
FIRNKANGGTTEYAASVKG

SEQ ID NO: 129 - protein, 10 aa, Artificial Sequence
  CEA CDR-H3
DRGLRFYFDY

SEQ ID NO: 130 - protein, 14 aa, Artificial Sequence
  CEA CDR-L1
TLRRGINVGAYSIY

SEQ ID NO: 131 - protein, 13 aa, Artificial Sequence
  CEA CDR-L2
YKSDSDKQQGSGV

SEQ ID NO: 132 - protein, 10 aa, Artificial Sequence
  CEA CDR-L3
MIWHSGASAV

SEQ ID NO: 133 - protein, 121 aa, Artificial Sequence
  CEA VH
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 134 - protein, 116 aa, Artificial Sequence
  CEA VL
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 135 - protein, 748 aa, Artificial Sequence
  His-tagged human FAP ECD
RPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 136 - protein, 761 aa, murine
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 137 - protein, 749 aa, Artificial Sequence
  Murine FAP ectodomain+poly-lys-tag+his6-tag
RPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGILSGRSQNHLYTHMTHFLKQCFSLSDGKKKKKKGHHHHHH

SEQ ID NO: 138 - protein, 748 aa, Artificial Sequence
  Cynomolgus FAP ectodomain+poly-lys-tag+his6-tag
RPPRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPFVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 139 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 140 - protein, 255 aa, Mus musculus
MAHLMTVQLLLLVMWMAECAQSRATRARTELLNVCMDAKHHKEKPGPEDNLHDQCSPWKTNSCCSTNTSQEAHKDISYLYRFNWNHCGTMTSECKRHFIQDTCLYECSPNLGPWIQQVDQSWRKERILDVPLCKEDCQQWWEDCQSSFTCKSNWHKGWNWSSGHNECPVGASCHPFTFYFPTSAALCEEIWSHSYKLSNYSRGSGRCIQMWFDPAQGNPNEEVARFYAEAMSGAGFHGTWPLLCSLSLVLLWVIS

SEQ ID NO: 141 - protein, 257 aa, Cynomolgus
MAQRMTTQLLLLLVWVAVVGEAQTRTARARTELLNVCMNAKHHKEKPGPEDKLHEQCRPWKKNACCSTNTSQEAHKDVSYLYRFNWNHCGEMAPACKRHFIQDTCLYECSPNLGPWIQQVDQSWRKERVLNVPLCKEDCERWWEDCRTSYTCKSNWHKGWNWTSGFNKCPVGAACQPFHFYFPTPTVLCNEIWTYSYKVSNYSRGSGRCIQMWFDPAQGNPNEEVARFYAAAMSGAGPWAAWPLLLSLALTLLWLLS

SEQ ID NO: 142 - protein, 2322 aa, Homo sapiens
MQSGPRPPLPAPGLALALTLTMLARLASAASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDPPHIIFPHGSLMVILEHTQKPLGPEVFQAYDPDSACEGLTFQVLGTSSGLPVERRDQPGEPATEFSCRELEAGSLVYVHRGGPAQDLTFRVSDGLQASPPATLKVVAIRPAIQIHRSTGLRLAQGSAMPILPANLSVETNAVGQDVSVLFRVTGALQFGELQKQGAGGVEGAEWWATQAFHQRDVEQGRVRYLSTDPQHHAYDTVENLALEVQVGQEILSNLSFPVTIQRATVWMLRLEPLHTQNTQQETLTTAHLEATLEEAGPSPPTFHYEVVQAPRKGNLQLQGTRLSDGQGFTQDDIQAGRVTYGATARASEAVEDTFRFRVTAPPYFSPLYTFPIHIGGDPDAPVLTNVLLVVPEGGEGVLSADHLFVKSLNSASYLYEVMERPRHGRLAWRGTQDKTTMVTSFTNEDLLRGRLVYQHDDSETTEDDIPFVATRQGESSGDMAWEEVRGVFRVAIQPVNDHAPVQTISRIFHVARGGRRLLTTDDVAFSDADSGFADAQLVLTRKDLLFGSIVAVDEPTRPIYRFTQEDLRKRRVLFVHSGADRGWIQLQVSDGQHQATALLEVQASEPYLRVANGSSLVVPQGGQGTIDTAVLHLDTNLDIRSGDEVHYHVTAGPRWGQLVRAGQPATAFSQQDLLDGAVLYSHNGSLSPRDTMAFSVEAGPVHTDATLQVTIALEGPLAPLKLVRHKKIYVFQGEAAEIRRDQLEAAQEAVPPADIVFSVKSPPSAGYLVMVSRGALADEPPSLDPVQSFSQEAVDTGRVLYLHSRPEAWSDAFSLDVASGLGAPLEGVLVELEVLPAAIPLEAQNFSVPEGGSLTLAPPLLRVSGPYFPTLLGLSLQVLEPPQHGALQKEDGPQARTLSAFSWRMVEEQLIRYVHDGSETLTDSFVLMANASEMDRQSHPVAFTVTVLPVNDQPPILTTNTGLQMWEGATAPIPAEALRSTDGDSGSEDLVYTIEQPSNGRVVLRGAPGTEVRSFTQAQLDGGLVLFSHRGTLDGGFRFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEANMFSVIIPMCLVLLLLALILPLLFYLRKRNKTGKHDVQVLTAKPRNGLAGDTETFRKVEPGQAIPLTAVPGQGPPPGGQPDPELLQFCRTPNPALKNGQYWV

SEQ ID NO: 143 - protein, 1210 aa, Homo sapiens [sequence omitted]

SEQ ID NO: 144 - protein, 1255 aa, Homo sapiens [sequence omitted]

SEQ ID NO: 145 - protein, 645 aa, Homo sapiens
MPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 146 - protein, 5 aa, Artificial Sequence
  Peptide linker G4S
GGGGS

SEQ ID NO: 147 - protein, 10 aa, Artificial Sequence
  Peptide linker (G4S)2
GGGGSGGGGS

SEQ ID NO: 148 - protein, 10 aa, Artificial Sequence
  Peptide linker (SG4)2
SGGGGSGGGG

SEQ ID NO: 149 - protein, 14 aa, Artificial Sequence
  Peptide linker G4(SG4)2
GGGGSGGGGSGGGG

SEQ ID NO: 150 - protein, 10 aa, Artificial Sequence
  Peptide linker
GSPGSSSSGS

SEQ ID NO: 151 - protein, 15 aa, Artificial Sequence
  Peptide linker (G4S)3
GGGGSGGGGSGGGGS

SEQ ID NO: 152 - protein, 20 aa, Artificial Sequence
  Peptide linker (G4S)4
GGGGSGGGGSGGGGSGGGGS

SEQ ID NO: 153 - protein, 8 aa, Artificial Sequence
  Peptide linker
GSGSGSGS

SEQ ID NO: 154 - protein, 8 aa, Artificial Sequence
  Peptide linker
GSGSGNGS

SEQ ID NO: 155 - protein, 8 aa, Artificial Sequence
  Peptide linker
GGSGSGSG

SEQ ID NO: 156 - protein, 6 aa, Artificial Sequence
  Peptide linker
GGSGSG

SEQ ID NO: 157 - protein, 4 aa, Artificial Sequence
  Peptide linker
GGSG

SEQ ID NO: 158 - protein, 8 aa, Artificial Sequence
  Peptide linker
GGSGNGSG

SEQ ID NO: 159 - protein, 8 aa, Artificial Sequence
  Peptide linker
GGNGSGSG

SEQ ID NO: 160 - protein, 6 aa, Artificial Sequence
  Peptide linker
GGNGSG

SEQ ID NO: 161 - protein, 420 aa, Artificial Sequence
  CEACAM5-based antigen Hu N(A2-B2)A-avi-His
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSALSPVVAKPQIKASKTTVTGDKDSVNLTCSTNDTGISIRWFFKNQSLPSSERMKLSQGNITLSINPVKREDAGTYWCEVFNPISKNQSDPIMLNVNYNALPQENLINVDGSGLNDIFEAQKIEWHEARAHHHHHH

SEQ ID NO: 162 - protein, 15 aa, Artificial Sequence
  avi-tag
GLNDIFEAQKIEWHE